(19) United States
(12) Patent Application Publication
(10) Pub. No.: US 2022/0177454 A1
(43) Pub. Date: Jun. 9, 2022

(54) TRISUBSTITUTED BENZENE COMPOUND HAVING NITROGEN-CONTAINING HETERO RING IN MOLECULAR TERMINUS AND ORGANIC ELECTROLUMINESCENCE ELEMENT

(71) Applicants: HODOGAYA CHEMICAL CO., LTD., Tokyo (JP); SHINSHU UNIVERSITY, Nagano (JP)

(72) Inventors: Kazunori TOGASHI, Tokyo (JP); Hideyoshi KITAHARA, Tokyo (JP); Musubu ICHIKAWA, Nagano (JP)

(73) Assignees: HODOGAYA CHEMICAL CO., LTD., Tokyo (JP); SHINSHU UNIVERSITY, Nagano (JP)

(21) Appl. No.: 17/435,417

(22) PCT Filed: Mar. 5, 2020
(86) PCT No.: PCT/JP2020/009437

§ 371 Date: Sep. 1, 2021

(30) Foreign Application Priority Data
Mar. 8, 2019 (JP) 2019-042267

(51) Int. Cl. C07D 403/10; H01L 51/00; C07D 493/00; C07D 403/14

(52) U.S. Cl. CPC C07D 403/10; H01L 51/0067; H01L 51/0072; H01L 51/0073; H01L 51/5072; C07D 403/14; H01L 51/0054; H01L 51/0056; C07D 493/00

(57) ABSTRACT

Provided are: a trisubstituted benzene compound represented by general formulae (1) and (2); and an organic electroluminescence (EL) element having a pair of electrodes and at least a pair of organic layers interposed therebetween, the organic EL element being characterized in that said compound is used as a constituent material for at least one organic layer.

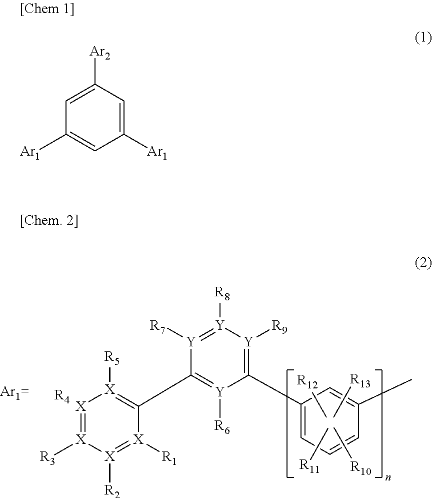

### TECHNICAL FIELD

The present invention relates to a compound suitable for an organic electroluminescence device which is a self-luminescent device suitable for various display devices, and the device, and specifically relates to a trisubstituted benzene compound that has two aromatic substituents having a nitrogen-containing heterocycle at a terminal and further has another aromatic substituent, and an organic EL device using the compound.

### BACKGROUND ART

Since organic electroluminescence devices are self-luminescent devices, they are bright and excellent in visibility as compared with liquid-crystalline devices, and capable of giving clear display. Therefore, the organic electroluminescence devices have been actively studied.

In 1987, C. W. Tang et al. of Eastman Kodak Company put an organic EL device using organic materials into practical use by developing a device having a multilayered structure in which various roles are assigned to respective materials. They formed a lamination of a fluorescent material capable of transporting electrons and an organic material capable of transporting holes, so that both charges are injected into the layer of the fluorescent material to emit light, thereby achieving a high luminance of 1,000 cd/m² or higher at a voltage of 10 V or lower (see, e.g., Patent Literatures 1 and 2).

To date, many improvements have been performed for practical utilization of the organic electroluminescence devices. High efficiency and durability have been achieved by an electroluminescent device in which an anode, a hole injection layer, a hole transport layer, a light emitting layer, an electron transport layer, an electron injection layer, and a cathode are sequentially provided on a substrate to further subdivide various roles (see, e.g., Non-Patent Literature 1).

Moreover, for the purpose of further improvements of luminous efficiency, utilization of triplet excitation has been attempted and utilization of a phosphorescent material has been investigated (see, e.g., Non-Patent Literature 2).

A device that utilizes light emission by thermally activated delayed fluorescence (TADF) has also been developed. In 2011, Adachi et al. of Kyushu University achieved an external quantum efficiency of 5.3% by a device using a thermally activated delayed fluorescence material (see, e.g., Non-Patent Literature 3), and achieved an external quantum efficiency exceeding 19% in 2012 (see, e.g., Non-Patent Literature 4).

The light emitting layer can also be prepared by doping a charge transporting compound generally referred to as a host material with a fluorescent material or a phosphorescent material. The selection of an organic material in an organic electroluminescence device greatly affects various properties such as efficiency and durability of the device.

In an organic electroluminescence device, an electron transport material having good electron injection and transport properties has been required for the purpose of achieving efficient charge recombination in a light emitting layer. In recent years, an electron transport material having a wider band gap (having a small electron affinity and a large work function) has been required for the purpose of improving the luminous efficiency of a blue device expected to have improved performance particularly.

When the electron affinity is small, an electron injection barrier to the light emitting layer particularly used in the blue device becomes small, and the electron injection efficiency to the light emitting layer is improved. Therefore, the luminous efficiency of the device can be expected to be improved.

When the work function is large, it is possible to prevent some of the holes from passing through the light emitting layer, and as a result, the probability of charge recombination in the light emitting layer is improved, and the luminous efficiency of the device can be expected to be improved.

Tris(8-hydroxyquinoline) aluminum (hereinafter, abbreviated as Alq₃), which is a typical light emitting material, is also generally used as an electron transport material. However, the work function is as small as 5.7 eV, and it cannot be said that the material has hole blocking property.

In the measures to prevent some of the holes from passing through the light emitting layer and improve the probability of charge recombination in the light emitting layer, there is a method of inserting a hole blocking layer. As the hole blocking material, a triazole (hereinafter, abbreviated as TAZ) derivative (see, e.g., Patent Literature 3), a bathocuproine (hereinafter, abbreviated as BCP), and the like have been proposed.

TAZ has a large work function of 6.3 eV and a high hole blocking ability, but has a large electron affinity of 2.7 eV. Therefore, the electron injection barrier to the light emitting layer is large, and it cannot be said that TAZ is sufficient for improving the efficiency of the device.

Furthermore, low electron transportability is a major problem in TAZ, and it is necessary to produce an organic electroluminescence device by combining TAZ with an electron transport material having higher electron transportability (see, e.g., Non-Patent Literature 5).

In addition, similarly to TAZ, BCP has a large work function of 6.7 eV and a high hole blocking ability, but has a large electron affinity of 2.8 eV. Therefore, the electron injection barrier to the light emitting layer is large, and it cannot be said that BCP is sufficient for improving the efficiency of the device.

In addition, since BCP has a low glass transition point (Tg) of 83° C., stability of a thin film thereof is poor, and it cannot be said that BCP sufficiently functions as a hole blocking layer.

As an electron transport material having high stability of a thin film and high electron injection and transport properties and a high hole blocking ability, 2,2′,2″-(1,3,5-phenylene)-tris(1-phenyl-1H-benzimidazole) (hereinafter, abbreviated as TPBi) has been proposed. (Patent Literature 4)

TPBi has a large work function of 6.2 eV and a high hole blocking ability, but has a large electron affinity of 2.7 eV. Therefore, the electron injection barrier to the light emitting layer is large, and it cannot be said that TPBi is sufficient for improving the efficiency of the device.

Any of these materials has a high hole blocking ability, but has a narrow band gap. Therefore, the electron injection barrier to the light emitting layer is large, and it cannot be said that the materials are sufficient for improving the efficiency of the blue device particularly. In order to improve the device properties of an organic electroluminescence device, an organic compound, which is excellent in electron injection and transport performance and hole blocking ability, and has a wider band gap, has been required.

### CITATION LIST

### Patent Literature

Patent Literature 1: JP-A-H8-48656
Patent Literature 2: Japanese Patent No. 3194657
Patent Literature 3: Japanese Patent No. 2734341
Patent Literature 4: Japanese Patent No. 3992794

### Non-Patent Literature

Non-Patent Literature 1: Japan Society of Applied Physics, 9th Class, Proceedings, Pages 55 to 61 (2001)
Non-Patent Literature 2: Japan Society of Applied Physics, 9th Class, Proceedings, Pages 23 to 31 (2001)
Non-Patent Literature 3: Appl. Phys. Let., 98, 083302 (2011)
Non-Patent Literature 4: Nature, 492,234 (2012)
Non-Patent Literature 5: Japan Society of Applied Physics, Organic Molecule and Bioelectronics Subcommittee Journal, Vol. 11, No. 1, Pages 13 to 19 (2000)

### SUMMARY OF INVENTION

### Technical Problem

An object of the present invention is to provide an organic compound having excellent electron injection and transport performance as a material for an organic electroluminescence device with low power consumption, and to provide an organic electroluminescence device with low power consumption using the compound. Examples of the physical properties of the organic compound suitable for the present invention include (1) a good electron injection and transport property, (2) a high hole blocking property, and (3) a wide band gap. Examples of the physical properties of the device suitable for the present invention include a low driving voltage and a high luminous efficiency.

### Solution to Problem

Therefore, in order to achieve the above object, the present inventors have designed a trisubstituted benzene compound that has two aromatic substituents in which a nitrogen-containing heterocycle having a large electron-withdrawing property is arranged at a terminal and which are linked to the meta-position, and has another aromatic substituent different from the above aromatic substituents for the purpose of improving the amorphous property of a molecule, experimentally produced various organic electroluminescence devices using the compound, and earnestly evaluated the properties of the device. As a result, they completed the present invention.

The trisubstituted benzene compound of the present invention capable of solving the above problems is represented by the following general formulas (1) and (2).

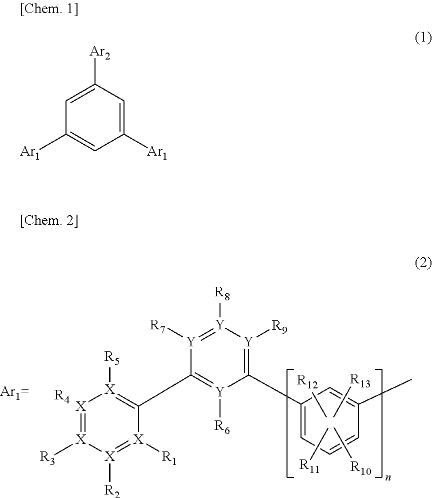

(In the formula, Ar₂ is different from Ar₁, and is an optionally substituted monovalent aromatic hydrocarbon group or aromatic heterocyclic group.

R₁ to R₁₃, which may be the same as or different from each other, each represent a hydrogen atom, a deuterium atom, a fluorine atom, a chlorine atom, a cyano group, a trifluoromethyl group, a linear or branched alkyl group having a carbon number of 1 to 8 which may have a substituent, a cycloalkyl group having a carbon number of 5 to 10 which may have a substituent, a linear or branched alkenyl group having a carbon number of 2 to 6 which may have a substituent, a linear or branched alkyloxy group having a carbon number of 1 to 6 which may have a substituent, or a cycloalkyloxy group having a carbon number of 5 to 10 which may have a substituent.

X's and Y's in Ar₁ each represent a carbon atom or a nitrogen atom, and at least one X represents a nitrogen atom.

In Ar₁, n represents an integer of 1 to 3.

Two Ar₁'s may be the same as or different from each other.)

An organic electroluminescence device of the present invention that can solve the above problems is an organic electroluminescence device including a pair of electrodes and at least one organic layer interposed therebetween, in which the at least one organic layer contains a trisubstituted benzene compound represented by the above general formulas (1) and (2) as a constituent material.

### Advantageous Effects of Invention

Since the compounds represented by the general formulas (1) and (2) of the present invention have a wide band gap and a small electron affinity, the electron injection barrier to a light emitting layer of a blue device is particularly small. Since the compounds have a large work function and a high hole blocking property, the compounds are useful as a constituent material of a hole blocking layer, an electron transport layer or a light emitting layer of an organic EL device. In the organic EL device produced using the compound of the present invention, the driving voltage can be lowered, and the luminous efficiency can be improved.

### BRIEF DESCRIPTION OF DRAWINGS

FIG. 1 This is a 1H-NMR chart of a compound (Compound 1) of Example 1.

FIG. 2 This is a 1H-NMR chart of a compound (Compound 4) of Example 2.

FIG. 3 This is a 1H-NMR chart of a compound (Compound 7) of Example 3.

FIG. 4 This is a 1H-NMR chart of a compound (Compound 19) of Example 4.

FIG. 5 This is a 1H-NMR chart of a compound (Compound 6) of Example 5.

FIG. 6 This is a 1H-NMR chart of a compound (Compound 194) of Example 6.

FIG. 7 This is a diagram illustrating a configuration of EL devices of Examples 10 to 20 and Comparative Example 1.

### DESCRIPTION OF EMBODIMENTS

Hereinafter, embodiments of the present invention will be described in detail. First, aspects of the present embodiment will be listed and described.

[1] A trisubstituted benzene compound represented by the following general formulas (1) and (2).

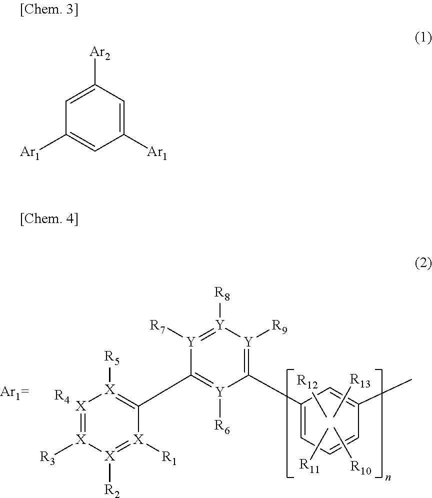

(In the formula, Ar₂ is different from Ar₁, and is an optionally substituted monovalent aromatic hydrocarbon group or aromatic heterocyclic group.

R₁ to R₁₃, which may be the same as or different from each other, each represent a hydrogen atom, a deuterium atom, a fluorine atom, a chlorine atom, a cyano group, a trifluoromethyl group, a linear or branched alkyl group having a carbon number of 1 to 8 which may have a substituent, a cycloalkyl group having a carbon number of 5 to 10 which may have a substituent, a linear or branched alkenyl group having a carbon number of 2 to 6 which may have a substituent, a linear or branched alkyloxy group having a carbon number of 1 to 6 which may have a substituent, or a cycloalkyloxy group having a carbon number of 5 to 10 which may have a substituent.

X's and Y's in Ar₁ each represent a carbon atom or a nitrogen atom, and at least one X represents a nitrogen atom.

In Ar₁, n represents an integer of 1 to 3.

Two Ar₁'s may be the same as or different from each other.)

[2] The trisubstituted benzene compound according to [1], in which Ar₁ in the general formula (1) is a substituent represented by any one of the following general formulas (3) to (6).

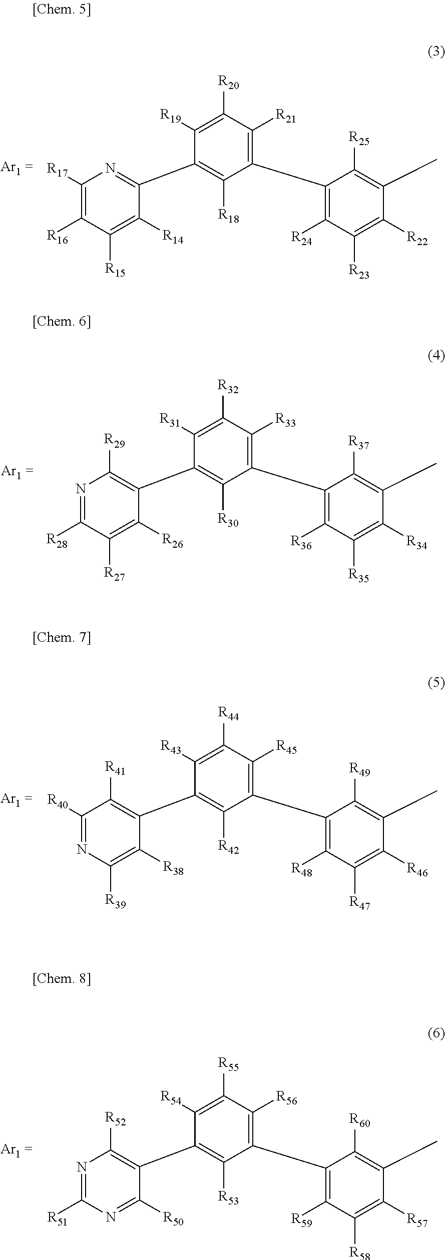

(In the formulas, R₁₀ to R₆₀, which may be the same as or different from each other, each represent a hydrogen atom, a deuterium atom, a fluorine atom, a chlorine atom, a cyano group, a trifluoromethyl group, a linear or branched alkyl group having a carbon number of 1 to 8 which may have a substituent, a cycloalkyl group having a carbon number of 5 to 10 which may have a substituent, a linear or branched alkenyl group having a carbon number of 2 to 6 which may have a substituent, a linear or branched alkyloxy group having a carbon number of 1 to 6 which may have a substituent, or a cycloalkyloxy group having a carbon number of 5 to 10 which may have a substituent.)
[3] An organic electroluminescence device including a pair of electrodes and at least one organic layer interposed therebetween, in which the at least one organic layer contains the trisubstituted benzene compound as described in [1] or [2] as a constituent material.
[4] The organic electroluminescence device according to [3], in which at least one layer of the organic layer containing the trisubstituted benzene compound as described in [1] or [2] is an electron transport layer.
[5] The organic electroluminescence device according to [3], in which at least one layer of the organic layer containing the trisubstituted benzene compound as described in [1] or [2] is a hole blocking layer.
[6] The organic electroluminescence device according to [3], in which at least one layer of the organic layer containing the trisubstituted benzene compound as described in [1] or [2] is an electron injection layer.
[7] The organic electroluminescence device according to [3], in which at least one layer of the organic layer containing the trisubstituted benzene compound as described in [1] or [2] is a hole injection layer.
[8] The organic electroluminescence device according to [3], in which at least one layer of the organic layer containing the trisubstituted benzene compound as described in [1] or [2] is a light emitting layer.

Specific examples of the aromatic hydrocarbon group or aromatic heterocyclic group represented by Ar₂ in the general formula (1) include a phenyl group, a biphenylyl group, a terphenylyl group, a naphthyl group, a phenanthrenyl group, a fluorenyl group, an indenyl group, a pyrenyl group, a perylenyl group, a fluoranthenyl group, a triphenylenyl group, a pyridyl group, a pyrimidinyl group, a triazinyl group, a furyl group, a pyrrolyl group, a thienyl group, a quinolyl group, an isoquinolyl group, a benzofuranyl group, a benzothienyl group, an indolyl group, a carbazolyl group, a benzoxazolyl group, a benzothiazolyl group, a quinoxalinyl group, a benzoimidazolyl group, a pyrazolyl group, a dibenzofuranyl group, a dibenzothienyl group, a naphthyridinyl group, a phenanthrolinyl group, an acridinyl group, a carbolinyl group, and the like.

Specific examples of the “substituent” in the aromatic hydrocarbon group or the aromatic heterocyclic group represented by Ar₂ in the general formula (1) include: a deuterium atom, a cyano group, or a nitro group; a halogen atom such as a fluorine atom, a chlorine atom, a bromine atom, and an iodine atom; a linear or branched alkyl group having a carbon number of 1 to 8 such as a methyl group, an ethyl group, an n-propyl group, an isopropyl group, an n-butyl group, an isobutyl group, a tert-butyl group, an n-pentyl group, an isopentyl group, a neopentyl group, an n-hexyl group, an isohexyl group, a neohexyl group, an n-heptyl group, an isoheptyl group, a neoheptyl group, an n-octyl group, an isooctyl group, and a neooctyl group; a linear or branched alkyloxy group having a carbon number of 1 to 8 such as a methyloxy group, an ethyloxy group, and a propyloxy group; an alkenyl group such as a vinyl group and an allyl group; an aryloxy group such as a phenyloxy group and a trilloxy group; an arylalkyloxy group such as a benzyloxy group and a phenethyloxy group; an aromatic hydrocarbon group such as a phenyl group, a biphenylyl group, a terphenylyl group, a naphthyl group, a phenanthrenyl group, a fluorenyl group, an indenyl group, a pyrenyl group, a peryleneyl group, a fluoranthenyl group, and a triphenylenyl group; an aromatic heterocyclic group such as a pyridyl group, a pyrimidinyl group, a triazinyl group, a thienyl group, a furyl group, a pyrrolyl group, a quinolyl group, an isoquinolyl group, a benzofuranyl group, a benzothienyl group, an indrill group, a carbazolyl group, a benzoxazolyl group, a benzothiazolyl group, a quinoxalinyl group, a benzoimidazolyl group, a pyrazolyl group, a dibenzofuranyl group, a dibenzothienyl group, and a carborinyl group; an aryl vinyl group such as a styryl group and a naphthyl vinyl group; an acyl group such as an acetyl group and a benzoyl group; and the like. These substituents may be further substituted with the substituent exemplified above. These substituents may be bonded to each other via a single bond, a substituted or unsubstituted methylene group, an oxygen atom or a sulfur atom to form a ring.

The carbon number of the “aromatic hydrocarbon group” of Ar₂ in the general formula (1) is, for example, 6 to 30. The carbon number of the “aromatic heterocyclic group” of Ar₂ in the general formula (1) is, for example, 2 to 20. Ar₂ in the general formula (1) may be a phenyl group, a phenanthrenyl group, a substituted or unsubstituted fluorenyl group, a triphenylenyl group, a pyridyl group, a substituted or unsubstituted pyrimidinyl group, a substituted or unsubstituted triazinyl group, a substituted indolyl group, a substituted or unsubstituted carbazolyl group, or a dibenzofuranyl group. Ar₂ in the general formula (1) may be a phenyl group, a pyridyl group, a pyrimidinyl group, or a dibenzofuranyl group.

Specific examples of the “linear or branched alkyl group having a carbon number of 1 to 8”, the “cycloalkyl group having a carbon number of 5 to 10”, or the “linear or branched alkenyl group having a carbon number of 2 to 6” in the “linear or branched alkyl group having a carbon number of 1 to 8 which may have a substituent”, the “cycloalkyl group having a carbon number of 5 to 10 which may have a substituent”, or the “linear or branched alkenyl group having a carbon number of 2 to 6 which may have a substituent” represented by R₁ to R₆₀ in the general formulas (2) to (6) include a methyl group, an ethyl group, an n-propyl group, an isopropyl group, an n-butyl group, an isobutyl group, a tert-butyl group, an n-pentyl group, an isopentyl group, a neopentyl group, an n-hexyl group, a cyclopentyl group, a cyclohexyl group, a 1-adamantyl group, a 2-adamantyl group, a vinyl group, an allyl group, an isopropenyl group, a 2-butenyl group, and the like.

In addition, specific examples of the “substituent” in the “linear or branched alkyl group having a carbon number of 1 to 8 which has a substituent”, the “cycloalkyl group having a carbon number of 5 to 10 which has a substituent”, or the “linear or branched alkenyl group having a carbon number of 2 to 6 which has a substituent” represented by R₁ to R₆₀ in the general formulas (2) to (6) include: a deuterium atom, a cyano group, or a nitro group; a halogen atom such as a fluorine atom, a chlorine atom, a bromine atom, and an iodine atom; a linear or branched alkyloxy group having a carbon number of 1 to 6 such as a methyloxy group, an ethyloxy group, and a propyloxy group; an alkenyl group such as a vinyl group and an allyl group; an aryloxy group such as a phenyloxy group and a trilloxy group; an arylalkyloxy group such as a benzyloxy group and a phenethyloxy group; an aromatic hydrocarbon group or a condensed polycyclic aromatic group such as a phenyl group, a biphenylyl group, a terphenylyl group, a naphthyl group, an anthracenyl group, a phenanthrenyl group, a fluorenyl group, an indenyl group, a pyrenyl group, a peryleneyl group, a fluoranthenyl group, and a triphenylenyl group; an aromatic heterocyclic group such as a pyridyl group, a pyrimidinyl group, a triazinyl group, a thienyl group, a furyl group, a pyrrolyl group, a quinolyl group, an isoquinolyl group, a benzofuranyl group, a benzothienyl group, an indrill group, a carbazolyl group, a benzoxazolyl group, a benzothiazolyl group, a quinoxalinyl group, a benzoimidazolyl group, a pyrazolyl group, a dibenzofuranyl group, a dibenzothienyl group, and a carborinyl group; and the like. These substituents may be further substituted with the substituent exemplified above. These substituents may be bonded to each other via a single bond, a substituted or unsubstituted methylene group, an oxygen atom or a sulfur atom to form a ring.

Specific examples of the “linear or branched alkyloxy group having a carbon number of 1 to 6” or the “cycloalkyloxy group having a carbon number of 5 to 10” in the “linear or branched alkyloxy group having a carbon number of 1 to 6 which may have a substituent” or the “cycloalkyloxy group having a carbon number of 5 to 10 which may have a substituent” represented by R₁ to R₆₀ in the general formulas (2) to (6) include a methyloxy group, an ethyloxy group, an n-propyloxy group, an isopropyloxy group, an n-butyloxy group, a tert-butyloxy group, an n-pentyloxy group, an n-hexyloxy group, a cyclopentyloxy group, a cyclohexyloxy group, a cycloheptyloxy group, a cyclooctyloxy group, a 1-adamantyloxy group, a 2-adamantyloxy group, and the like. These groups may be bonded to each other via a single bond, a substituted or unsubstituted methylene group, an oxygen atom or a sulfur atom to form a ring.

Examples of the “substituent” in the “linear or branched alkyloxy group having a carbon number of 1 to 6 which has a substituent” or the “cycloalkyloxy group having a carbon number of 5 to 10 which has a substituent” represented by R₁ to R₆₀ in the general formulas (2) to (6) include the same groups as those exemplified for the “substituent” in the “linear or branched alkyl group having a carbon number of 1 to 8 which has a substituent”, the “cycloalkyl group having a carbon number of 5 to 10 which has a substituent”, or the “linear or branched alkenyl group having a carbon number of 2 to 6 which has a substituent” represented above, and the same forms can also be exemplified.

Here, Ar₁ in the general formula (1) may be a substituent represented by the general formula (4) or (6). Also in this case, two Ar₁'s may be the same as or different from each other. In this case, R₂₆ to R₃₇ in the general formula (4) may be the same as or different from each other, and each may be a hydrogen atom, a deuterium atom, a fluorine atom, a chlorine atom, a cyano group, a trifluoromethyl group, an unsubstituted linear or branched alkyl group having a carbon number of 1 to 8, an unsubstituted cycloalkyl group having a carbon number of 5 to 10, an unsubstituted linear or branched alkenyl group having a carbon number of 2 to 6, an unsubstituted linear or branched alkyloxy group having a carbon number of 1 to 6, or an unsubstituted cycloalkyloxy group having a carbon number of 5 to 10. In this case, R₂₆ to R₃₇ in the general formula (4) may be a hydrogen atom. In this case, R₅₀ to R₆₀ in the general formula (6) may be the same as or different from each other, and each may be a hydrogen atom, a deuterium atom, a fluorine atom, a chlorine atom, a cyano group, a trifluoromethyl group, an unsubstituted linear or branched alkyl group having a carbon number of 1 to 8, an unsubstituted cycloalkyl group having a carbon number of 5 to 10, an unsubstituted linear or branched alkenyl group having a carbon number of 2 to 6, an unsubstituted linear or branched alkyloxy group having a carbon number of 1 to 6, or an unsubstituted cycloalkyloxy group having a carbon number of 5 to 10. In this case, R₅₀ to R₆₀ in the general formula (6) may be a hydrogen atom.

The compounds represented by the general formulas (1) to (6) of the present embodiment are novel compounds, and have a wider band gap and a smaller electron affinity than conventional electron transport materials. Therefore, the electron injection barrier to the light emitting layer of the blue device is particularly small. Furthermore, since the work function is large, the hole blocking property is high. Therefore, the device using the compound of the present embodiment has a function of lowering the driving voltage and improving the luminous efficiency.

The compounds represented by the general formulas (1) to (6) of the present embodiment can be used as a constituent material of a hole blocking layer and/or an electron transport layer of an organic EL device. The compounds have a function that, by using the material having a wider band gap and a smaller electron affinity than conventional materials, the efficiency of injecting and transporting electrons from the hole blocking layer or the electron transport layer to the light emitting layer is improved, and an organic EL device having improved driving voltage and luminous efficiency can be achieved.

The compounds represented by the general formulas (1) to (6) of the present embodiment can also be used as a constituent material of a light emitting layer of an organic EL device. The compounds have a function that, by using the material of the present embodiment, which has a more excellent electron transportability and a wider band gap than conventional materials, in the light emitting layer as a host material of the light emitting layer so as to support a fluorescent material or a phosphorescent material called a dopant, an organic EL device in which the driving voltage is lowered and the luminous efficiency is improved can be achieved.

The compounds represented by the general formulas (1) to (6) of the present embodiment are novel compounds, and these compounds can be synthesized, for example, as follows. The compound can be synthesized by subjecting a benzene halogenated at 1, 3 and 5 positions and a boronic acid or boric acid ester having various aromatic substituents to Suzuki-Miyaura coupling reaction, and further subjecting the synthesized halogen compound and another boronic acid or boric acid ester having an aromatic substituent having a nitrogen-containing heterocycle disposed at the terminal and linked to the meta-position to Suzuki-Miyaura coupling reaction.

Specific examples of preferable compounds among the compounds represented by the general formulas (1) to (6) are shown below, but the present invention is not limited to these compounds.

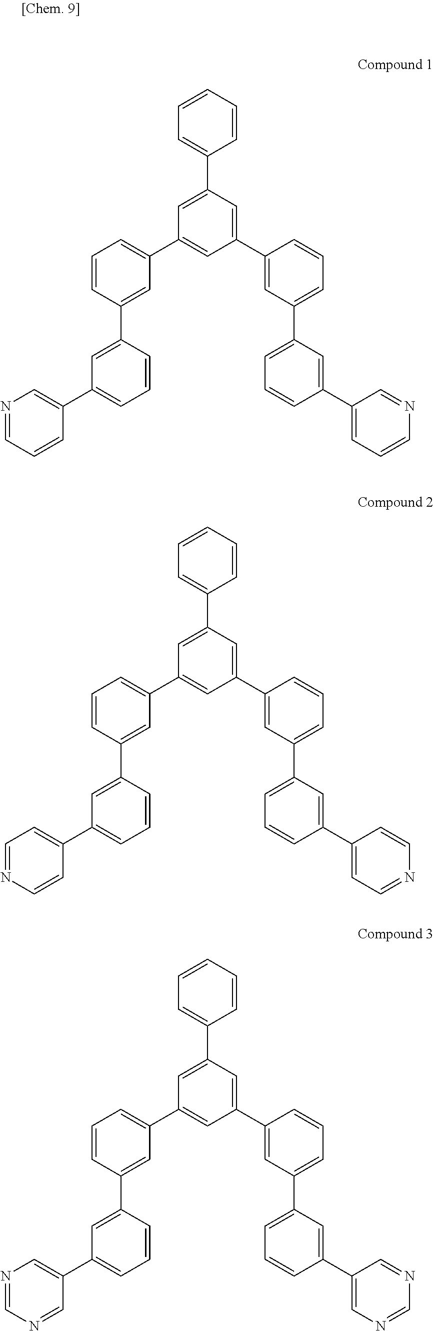

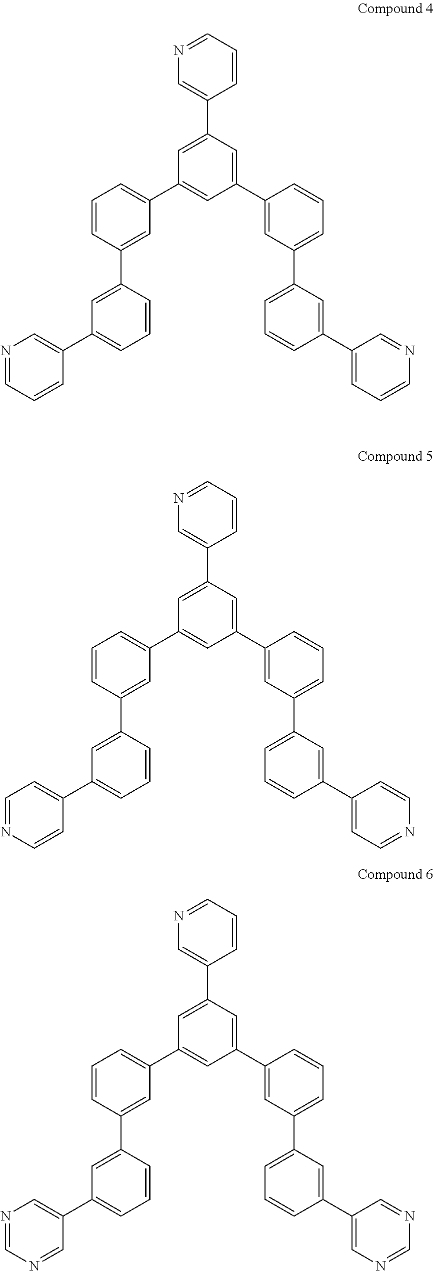

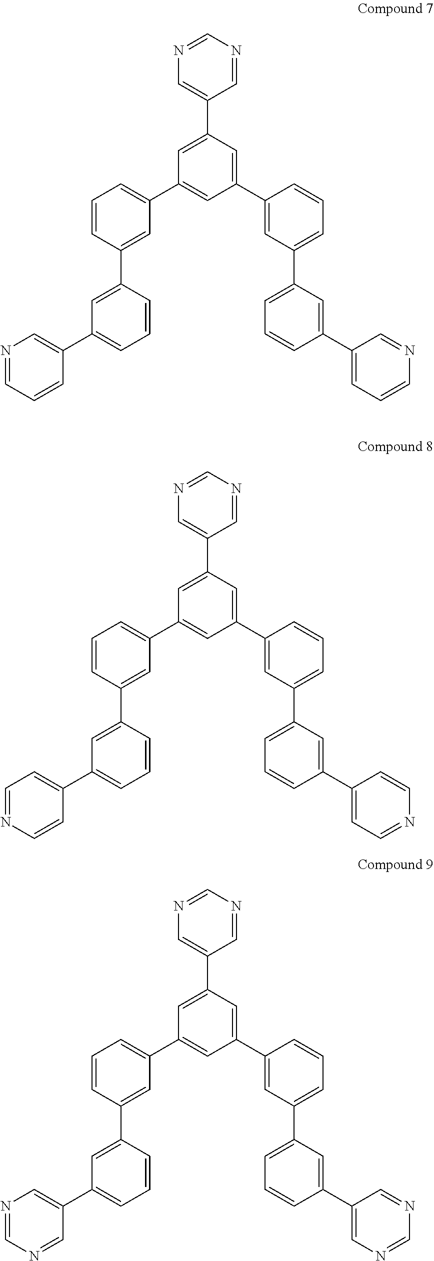

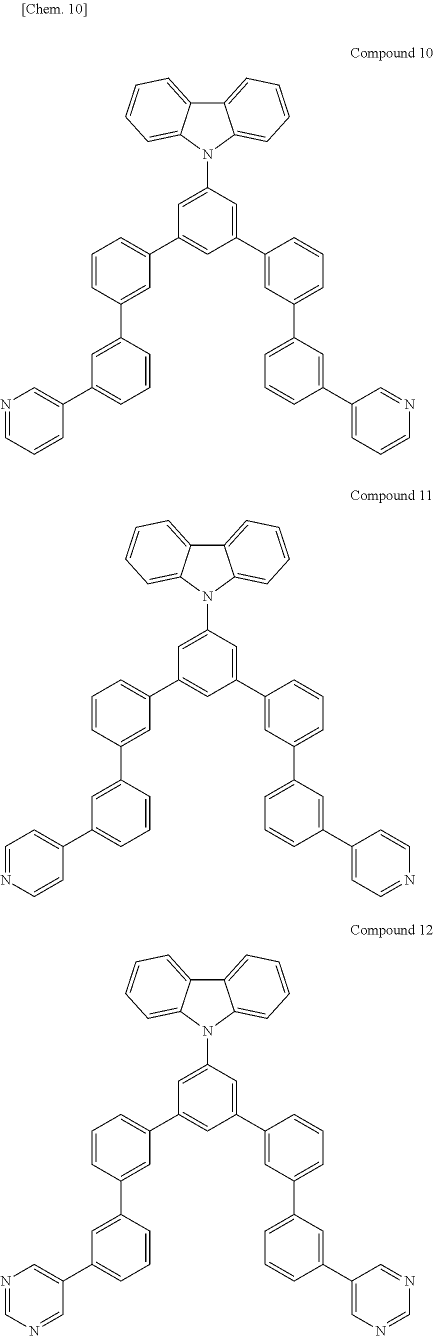

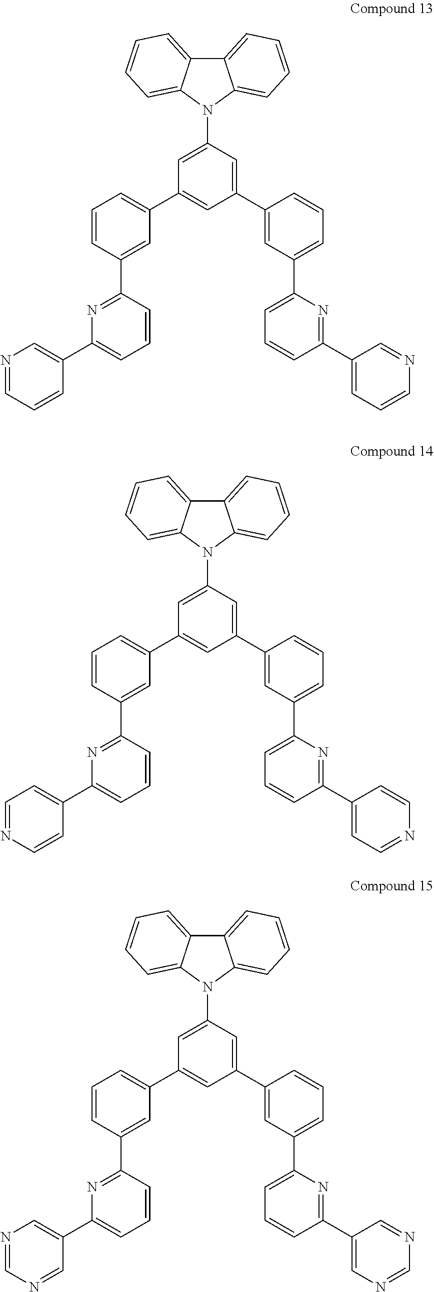

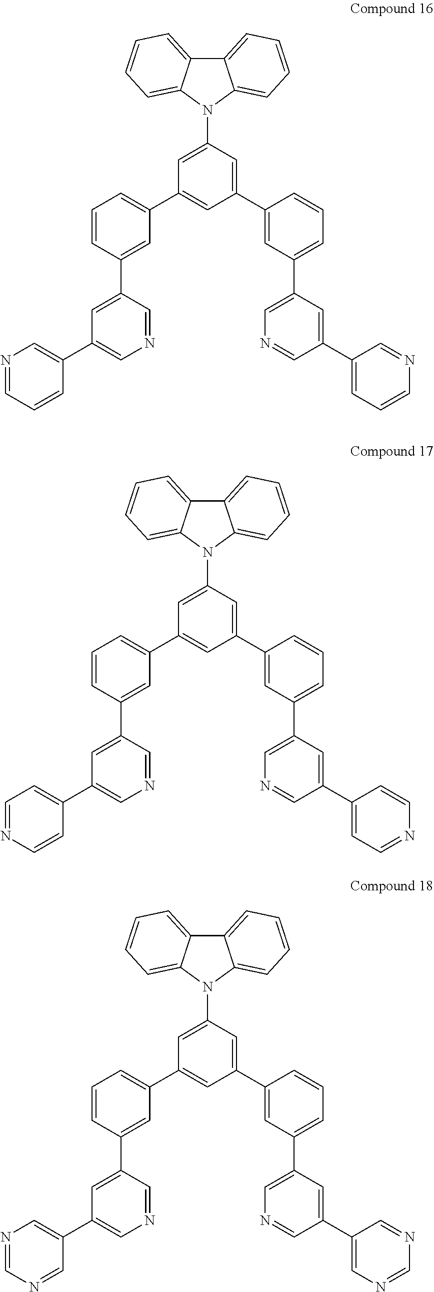

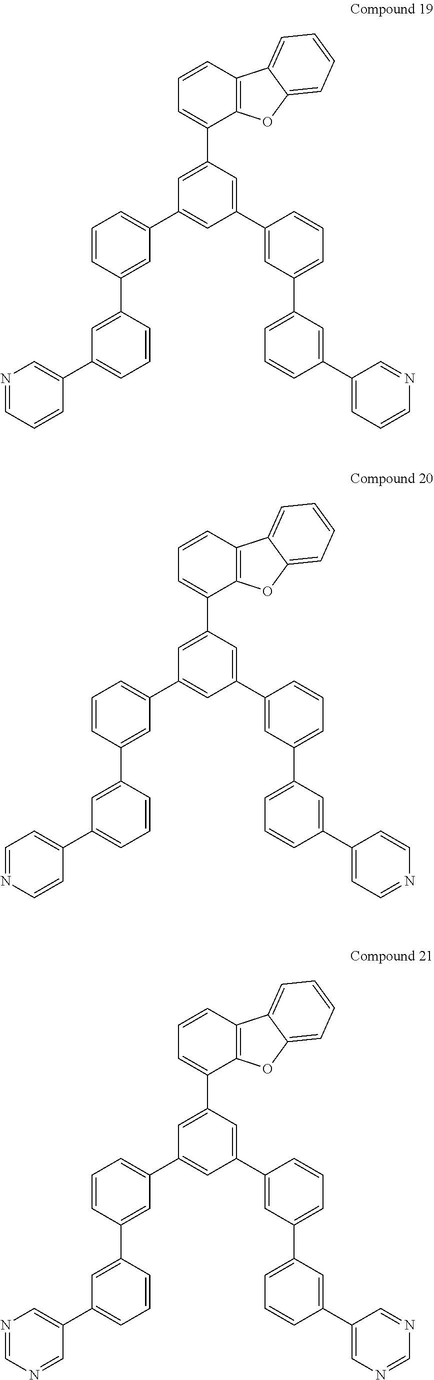

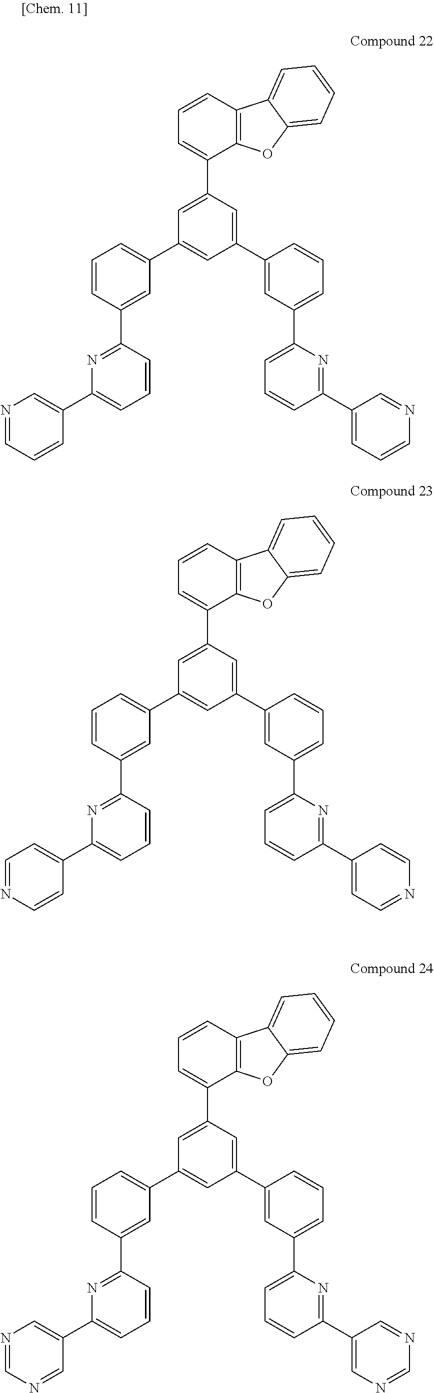

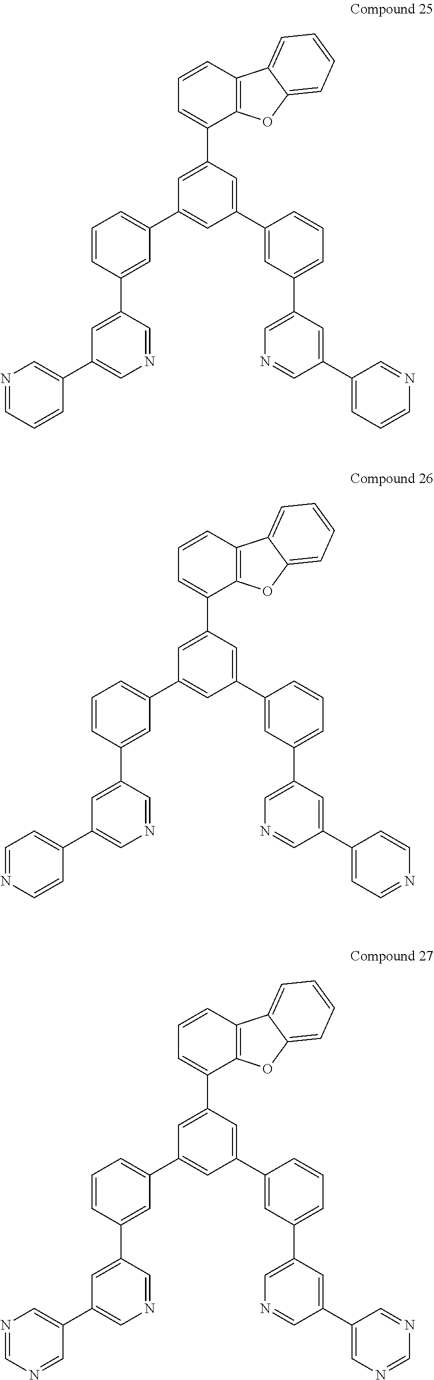

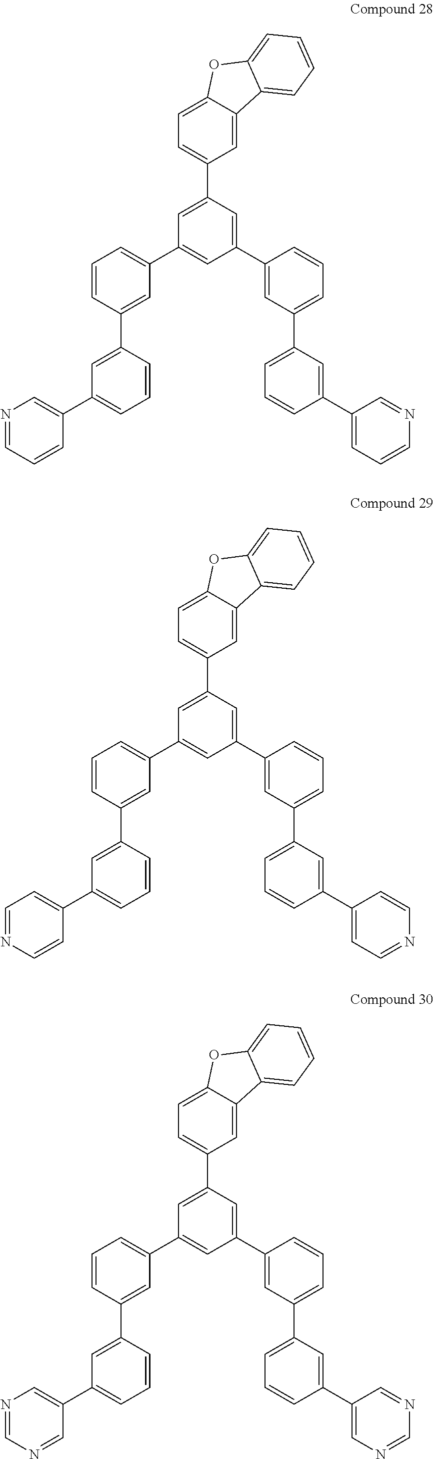

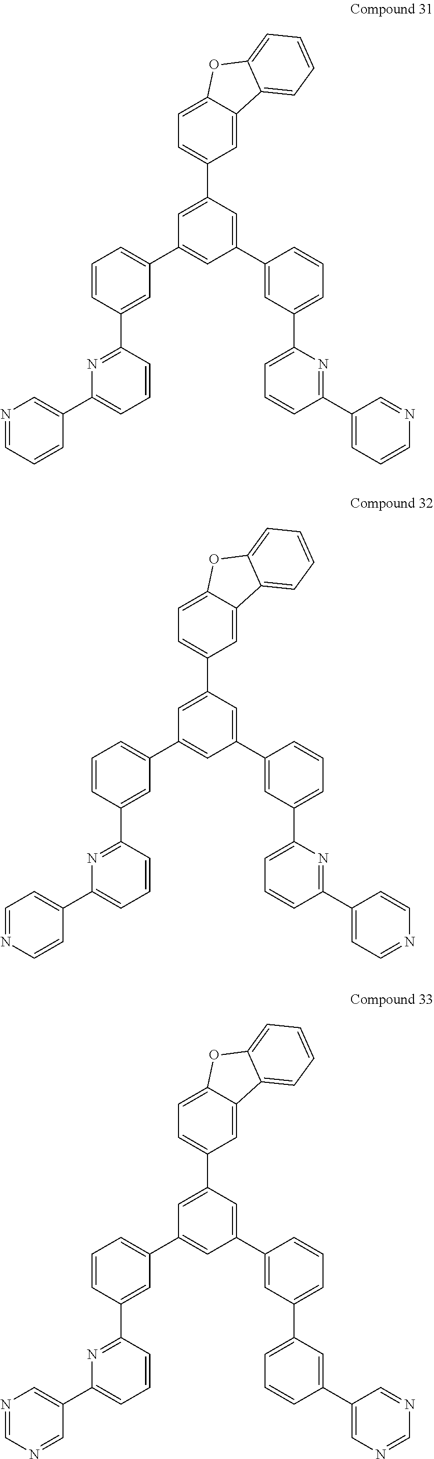

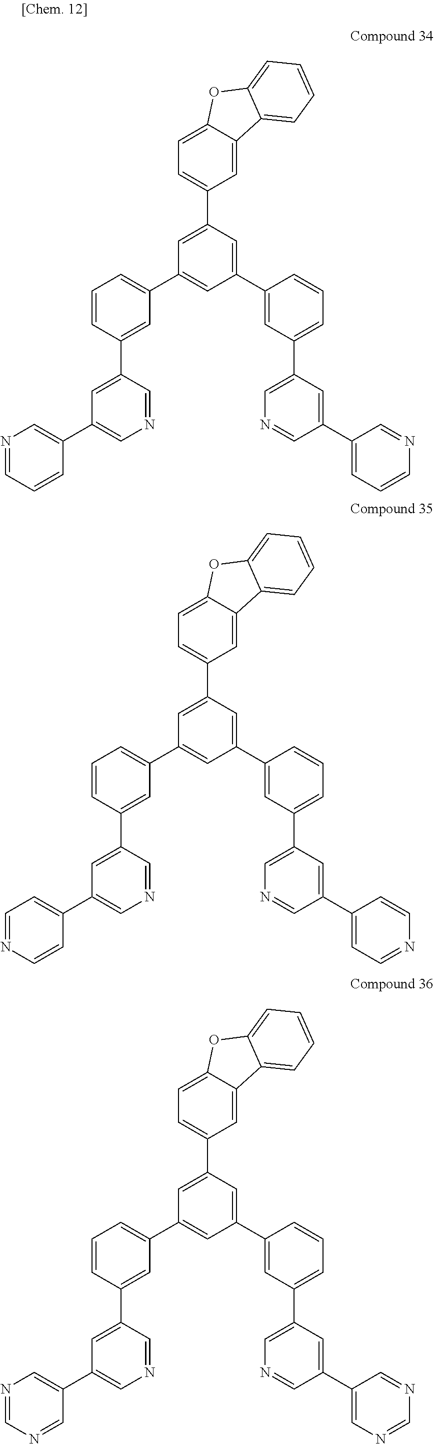

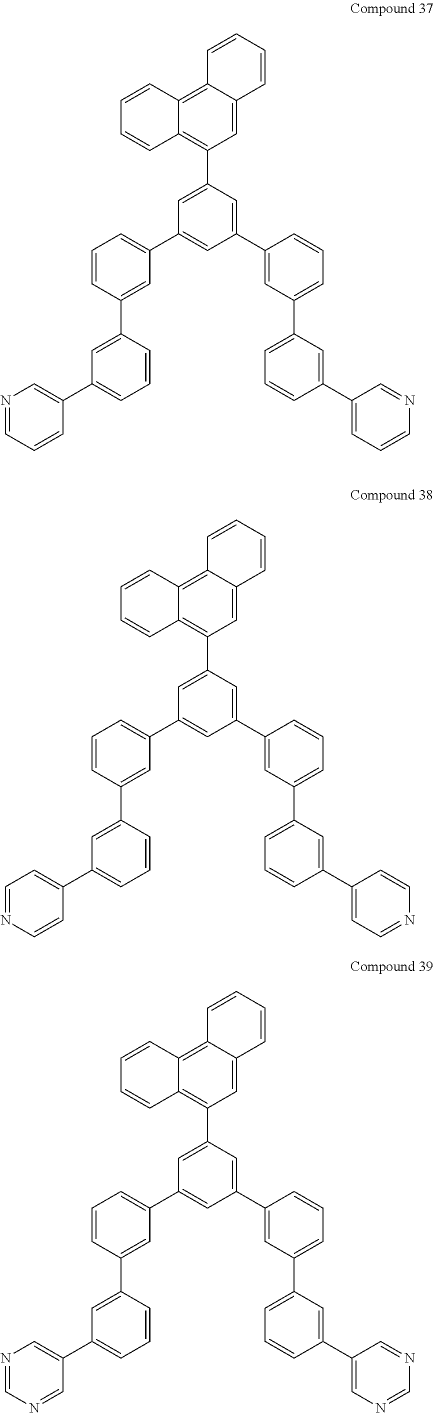

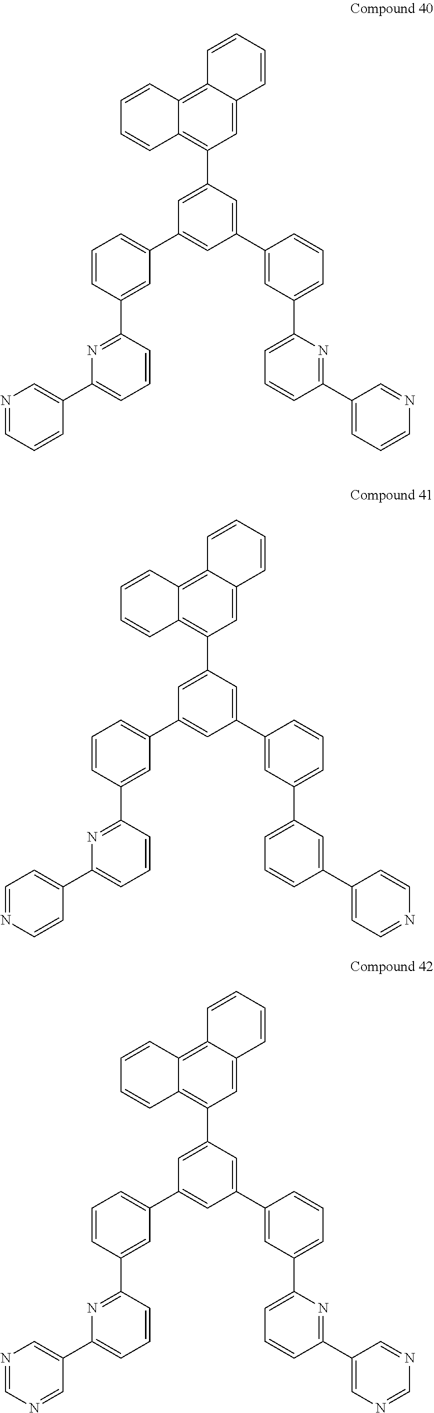

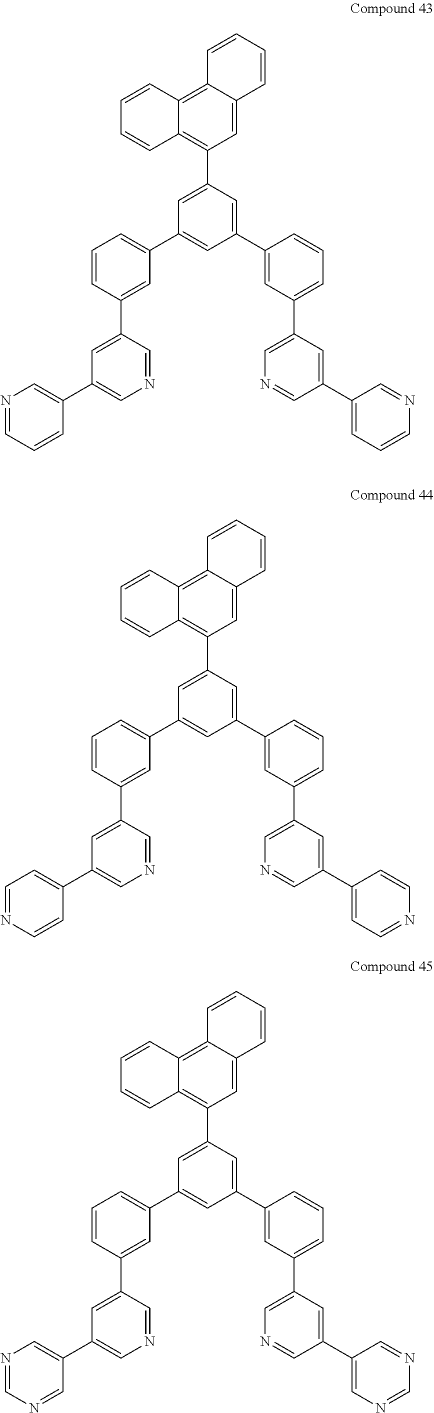

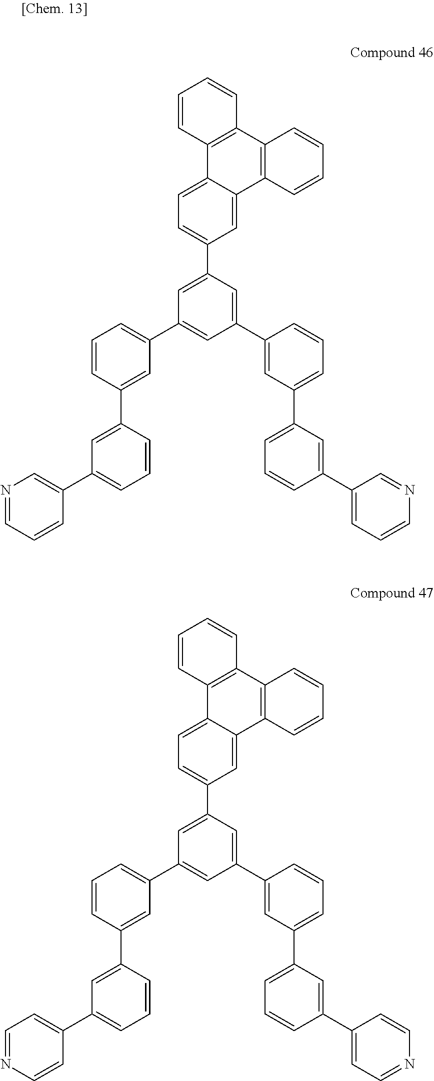

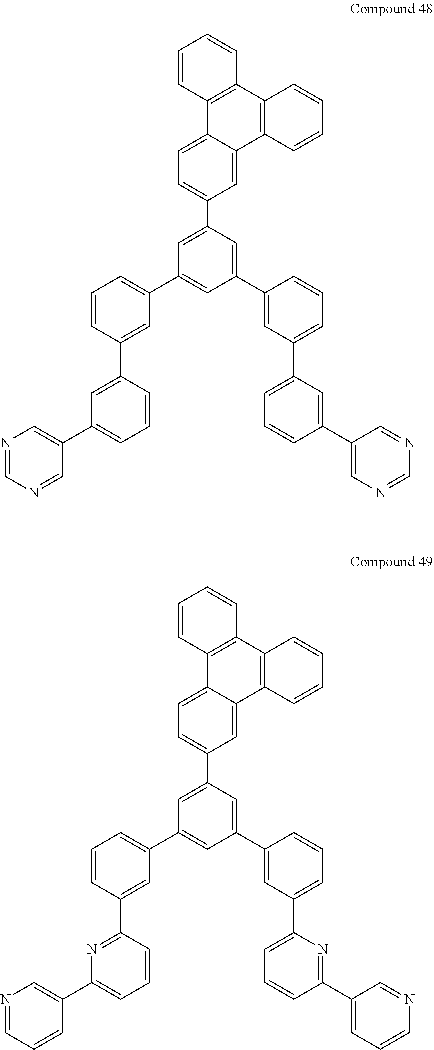

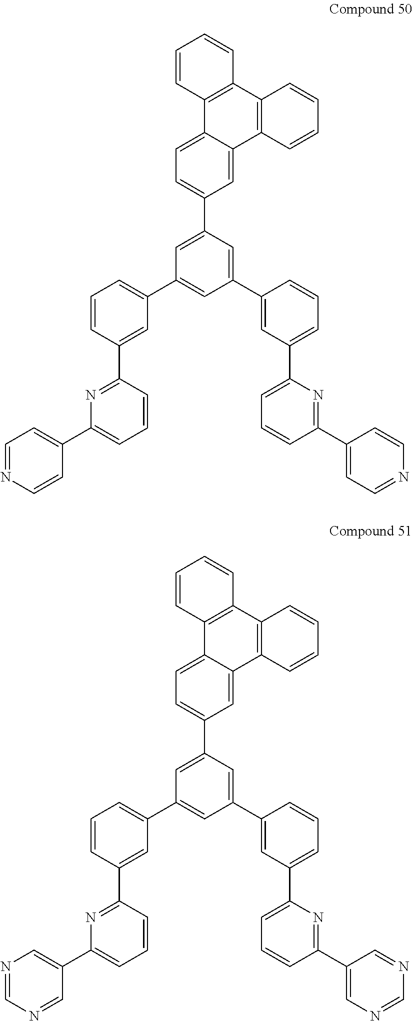

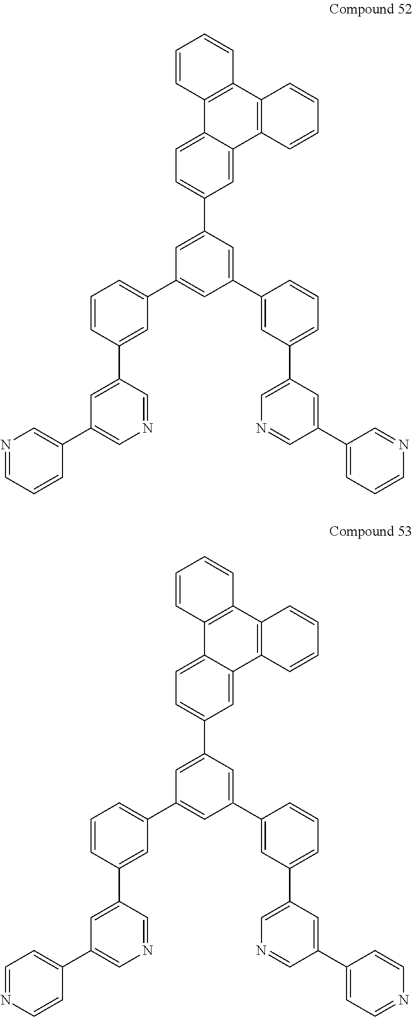

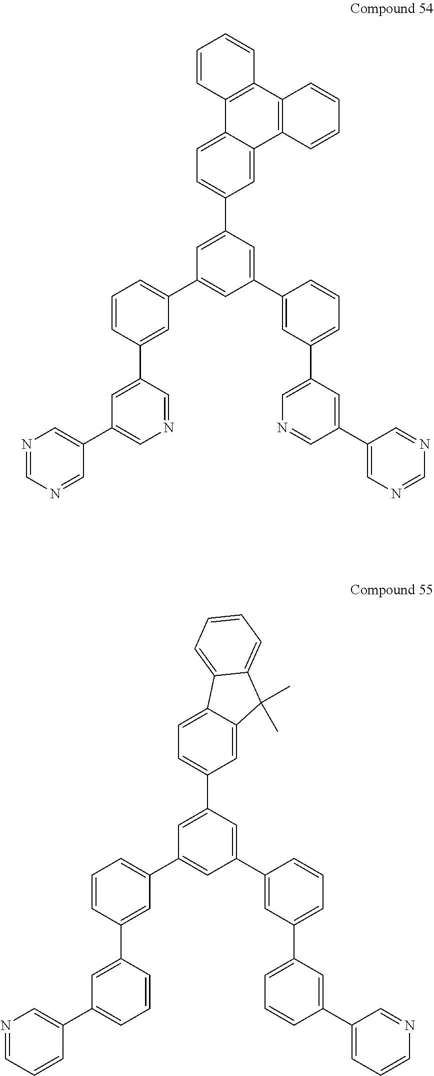

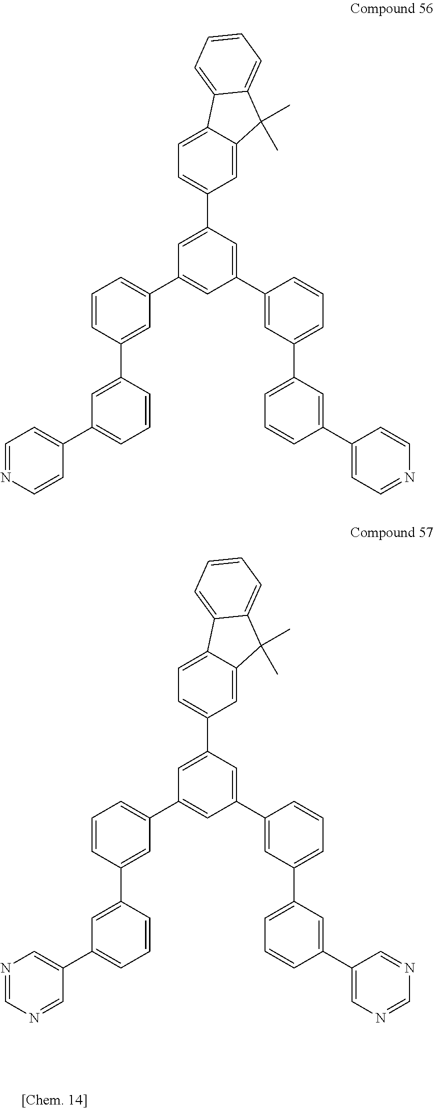

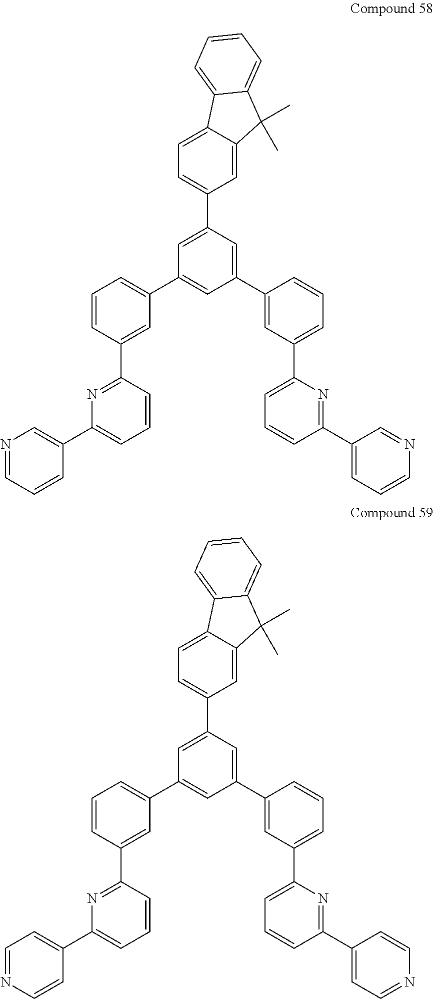

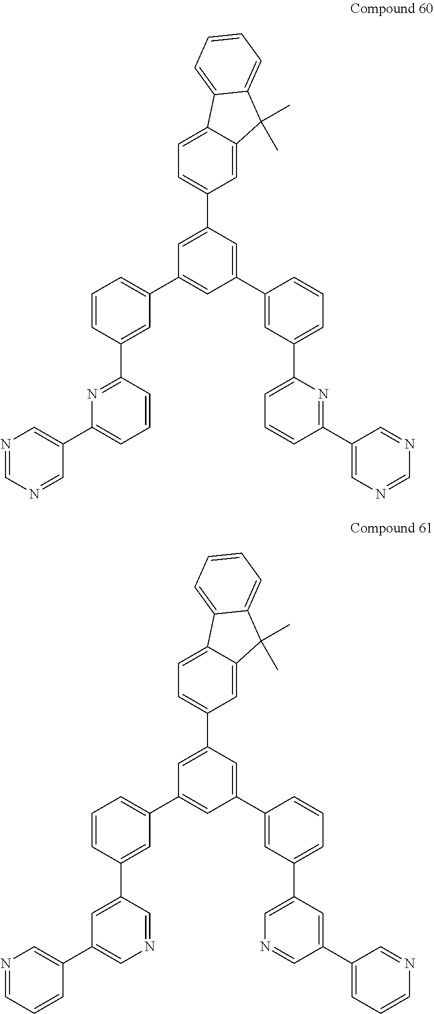

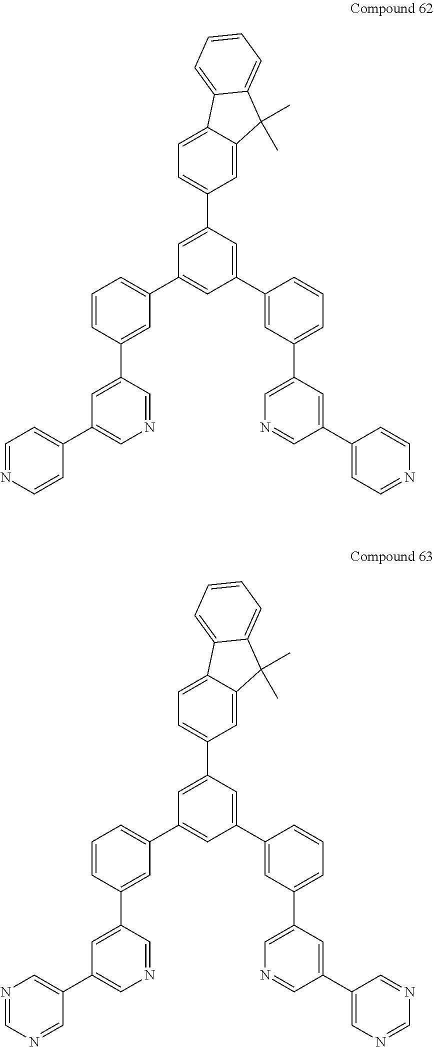

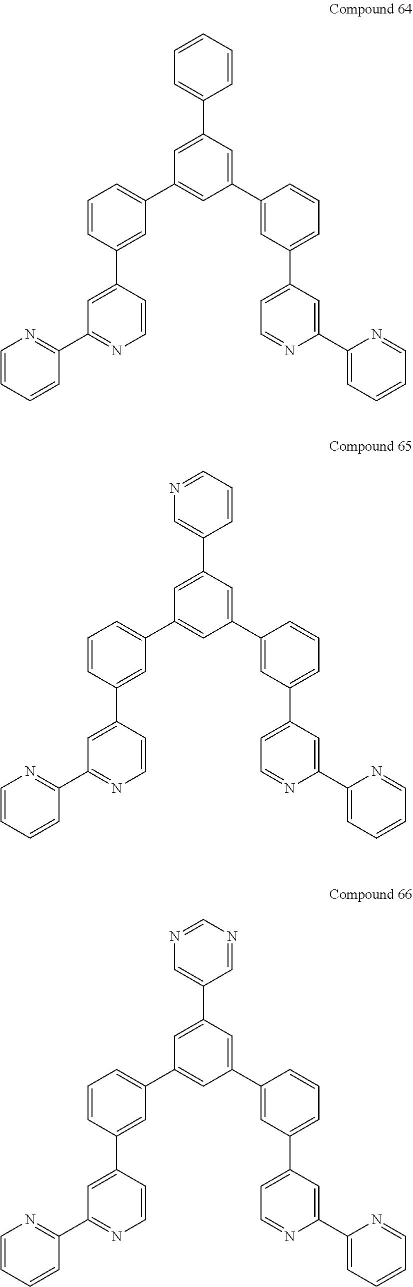

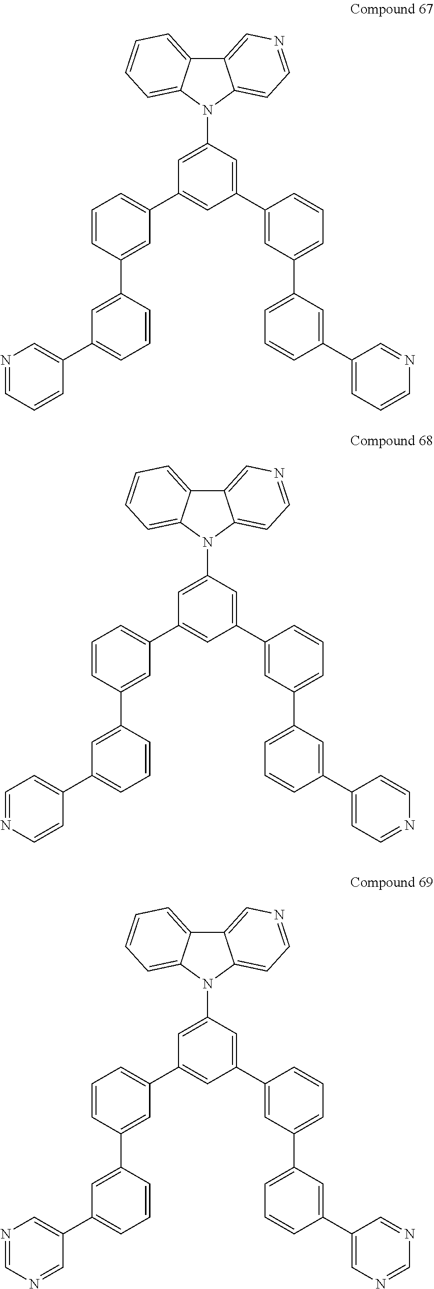

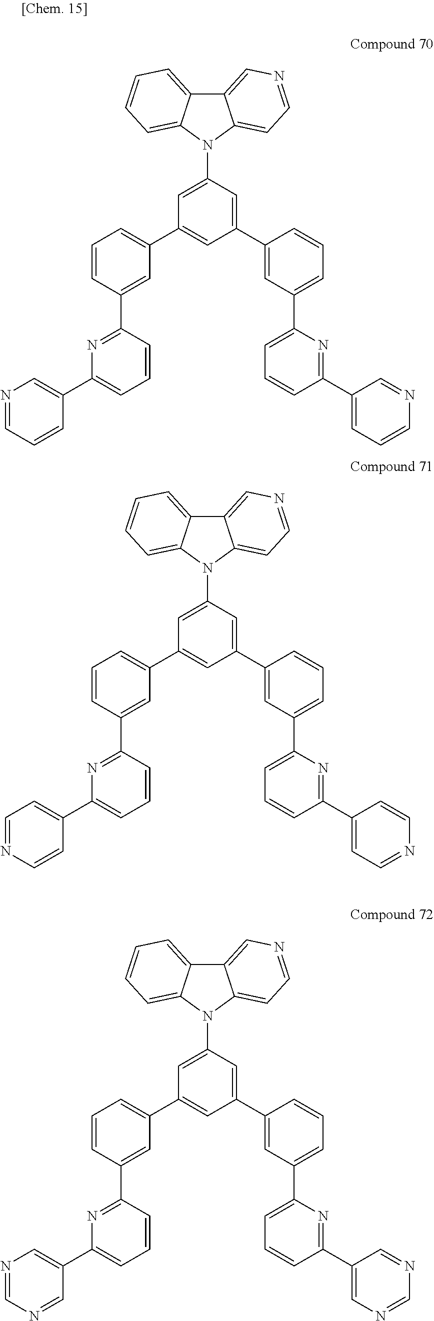

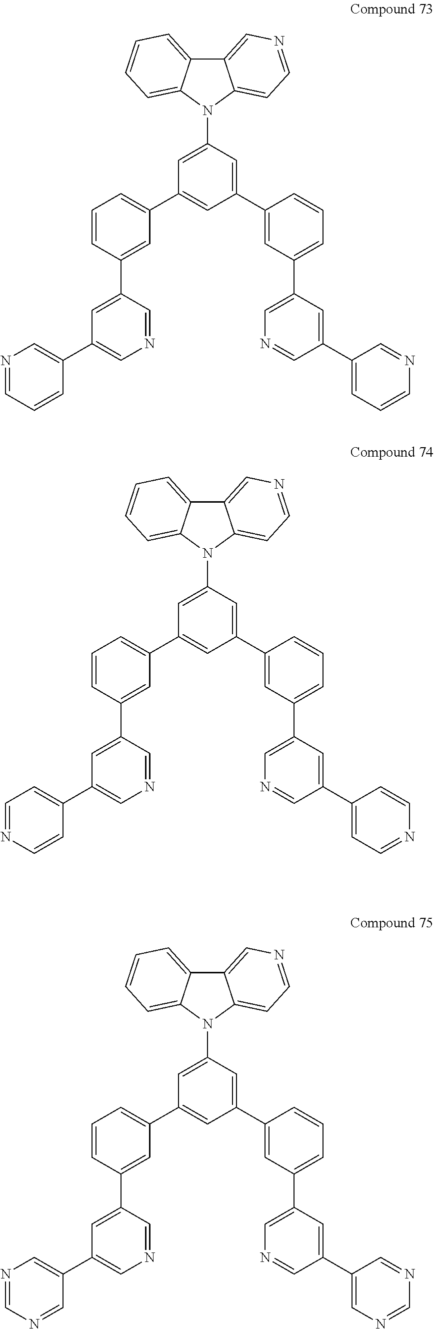

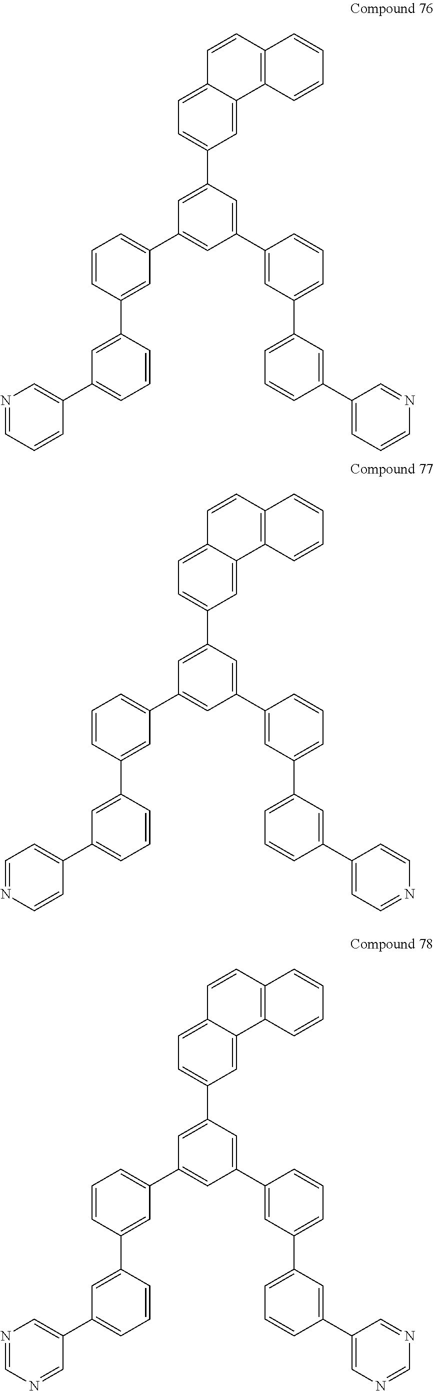

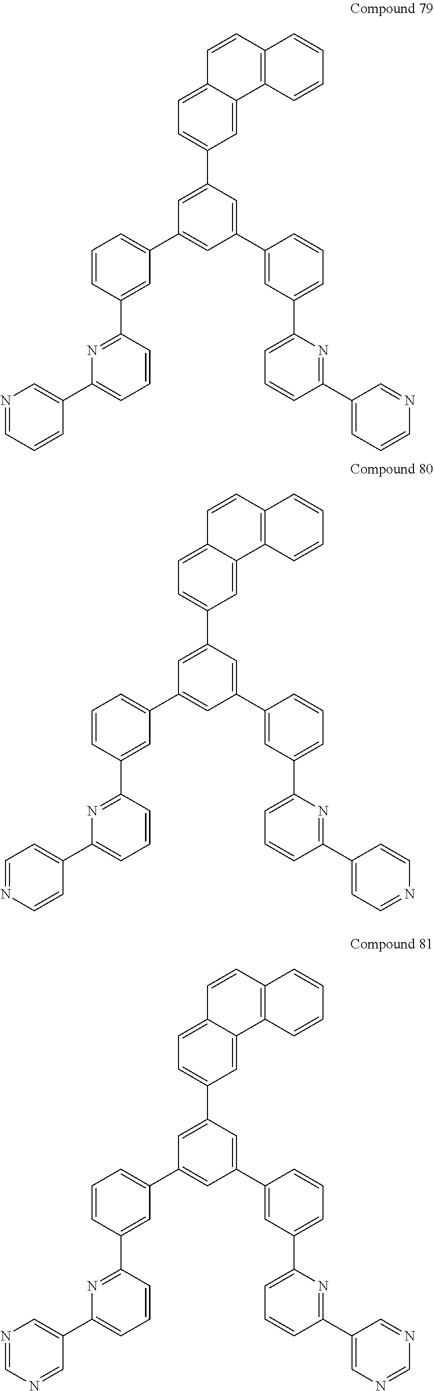

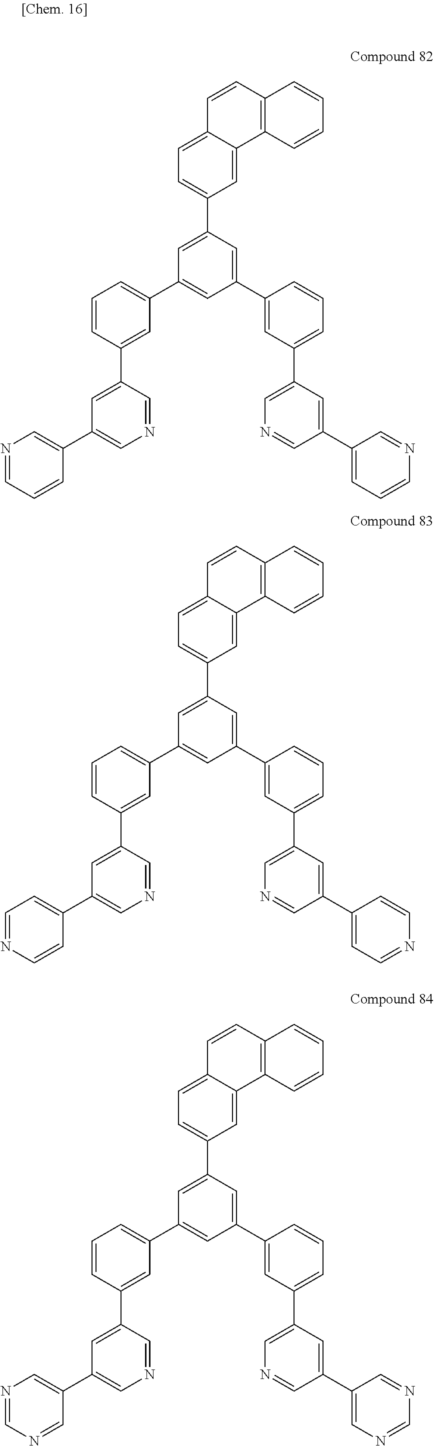

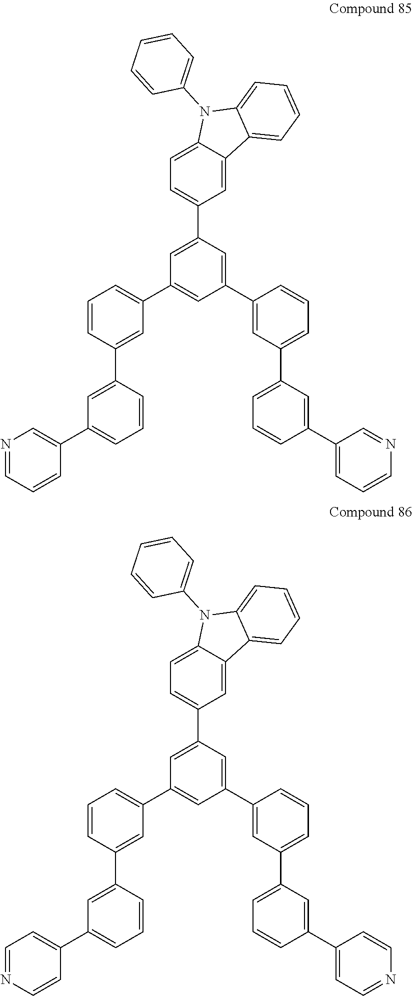

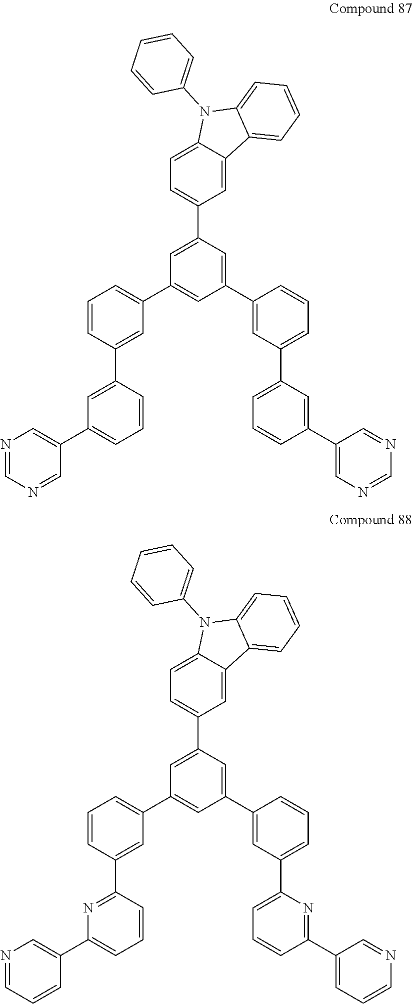

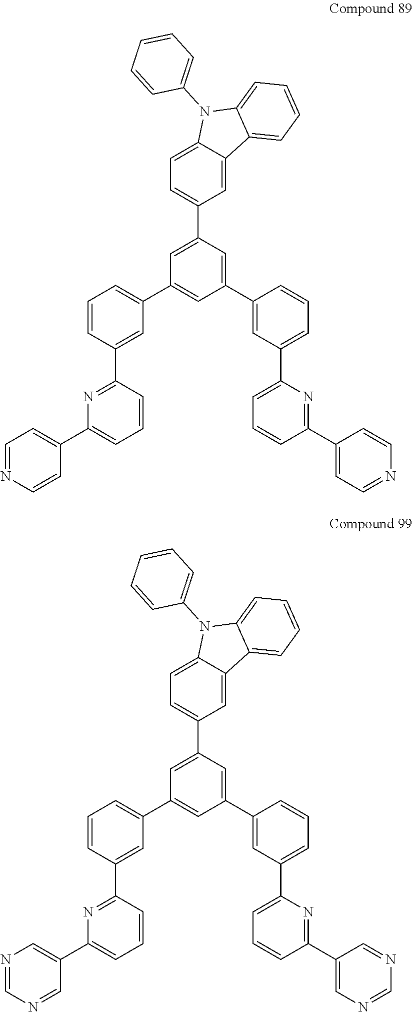

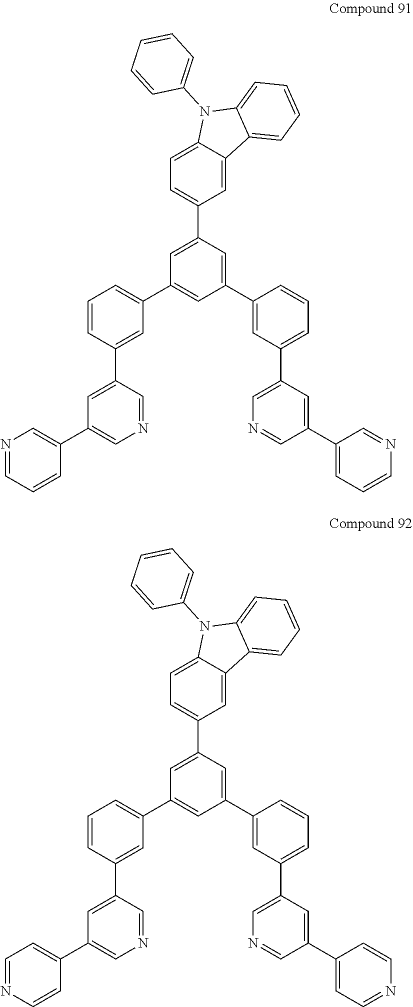

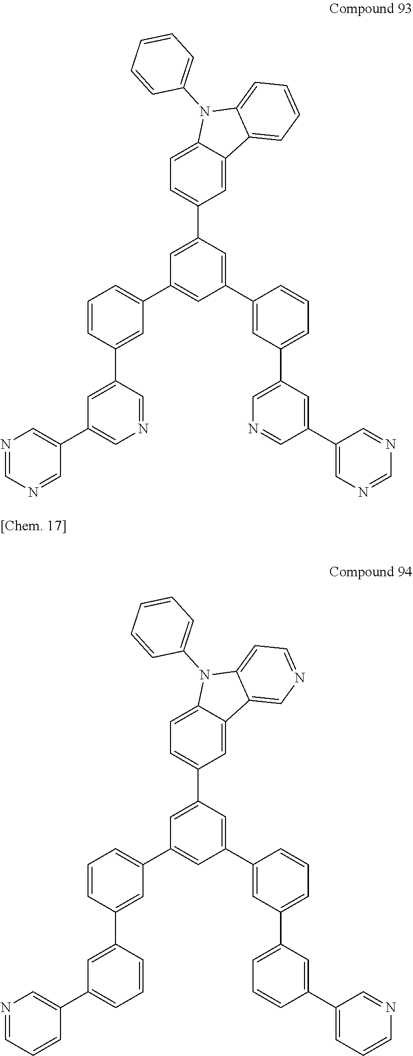

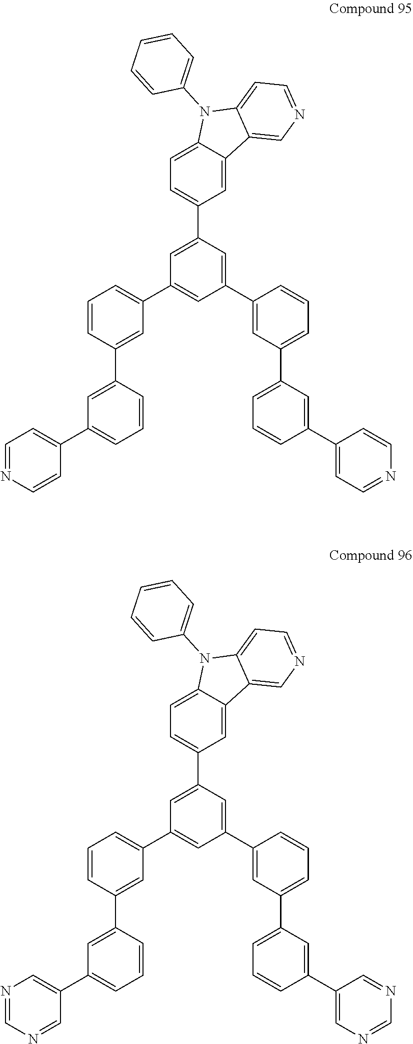

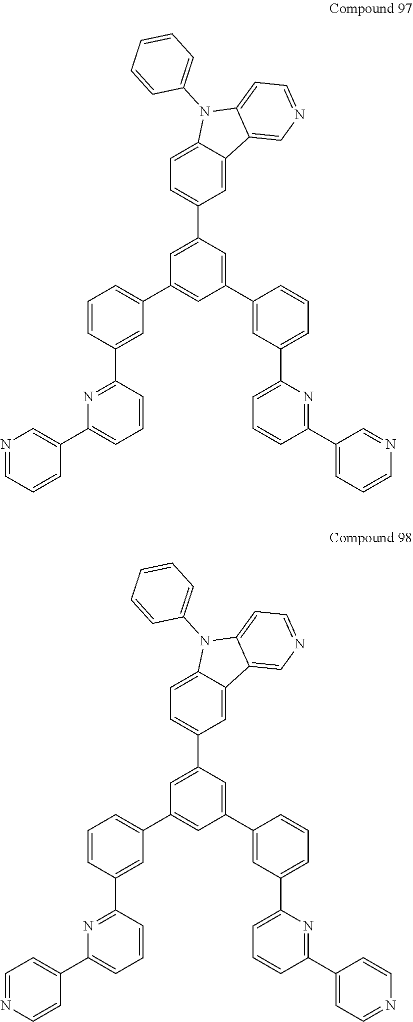

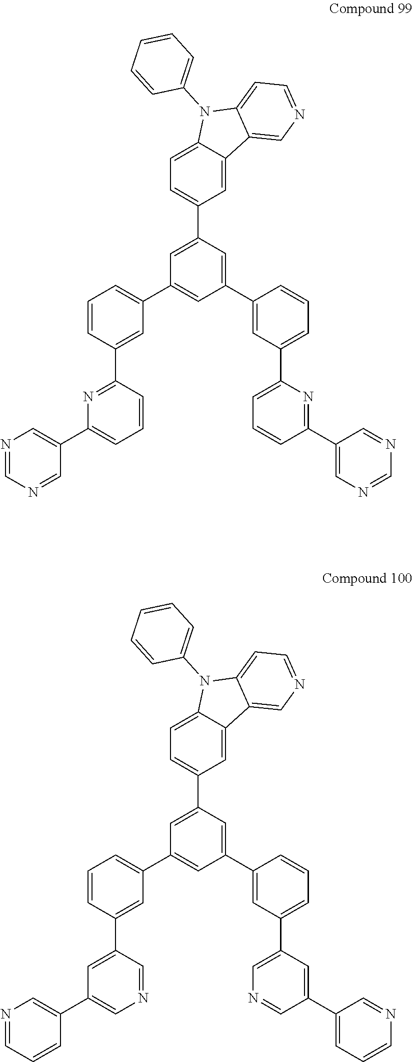

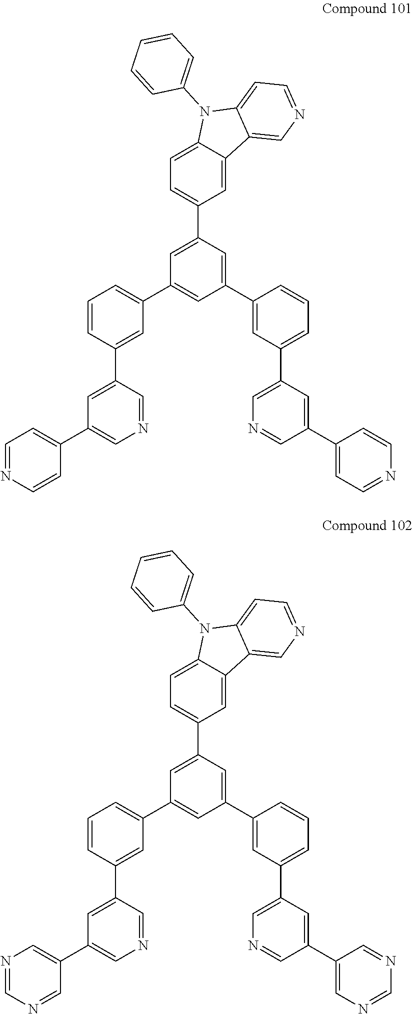

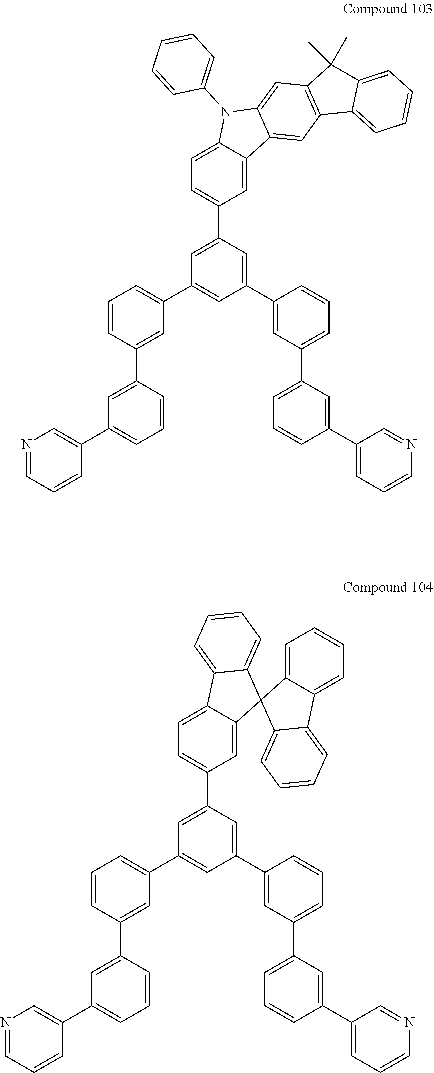

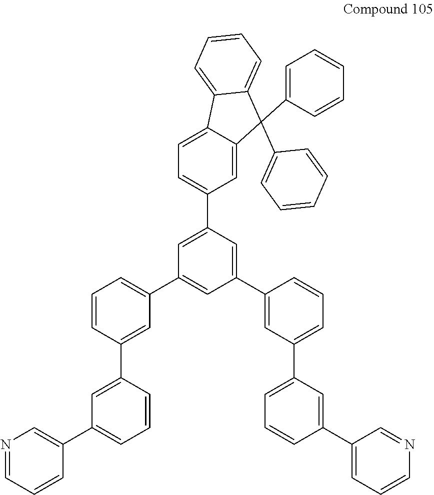

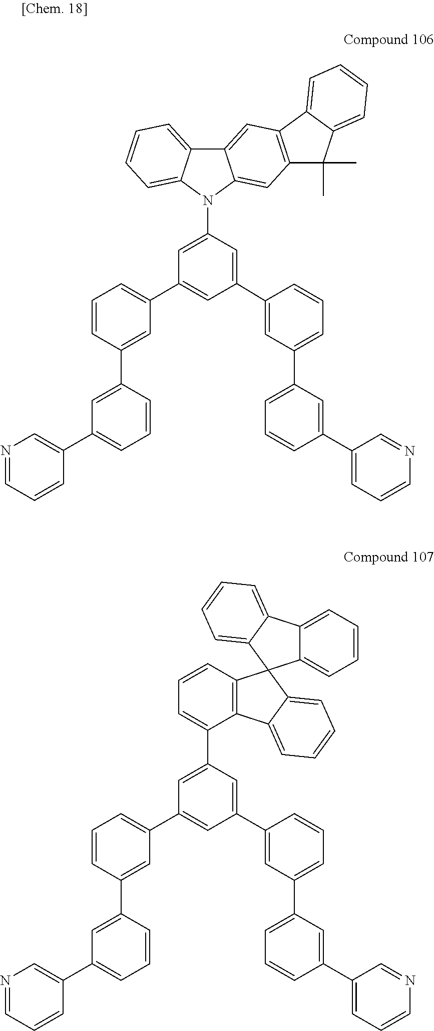

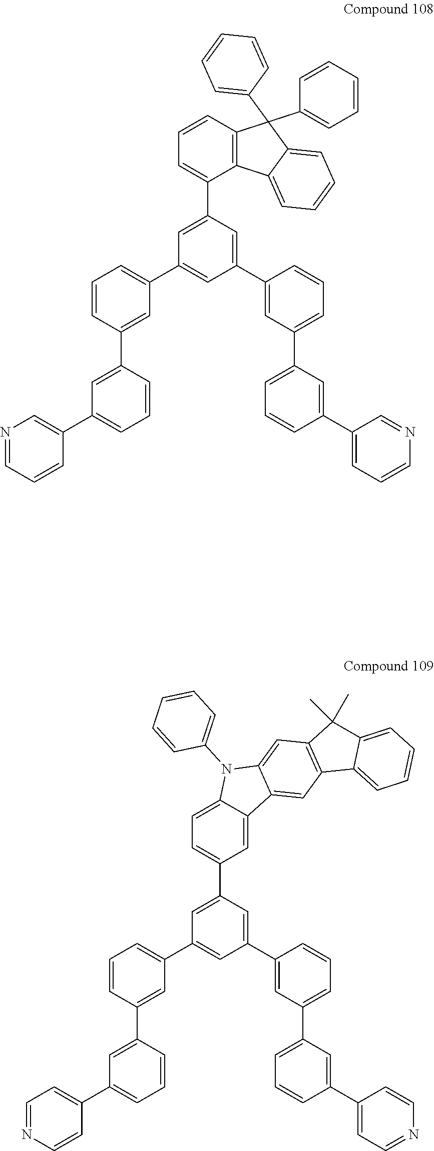

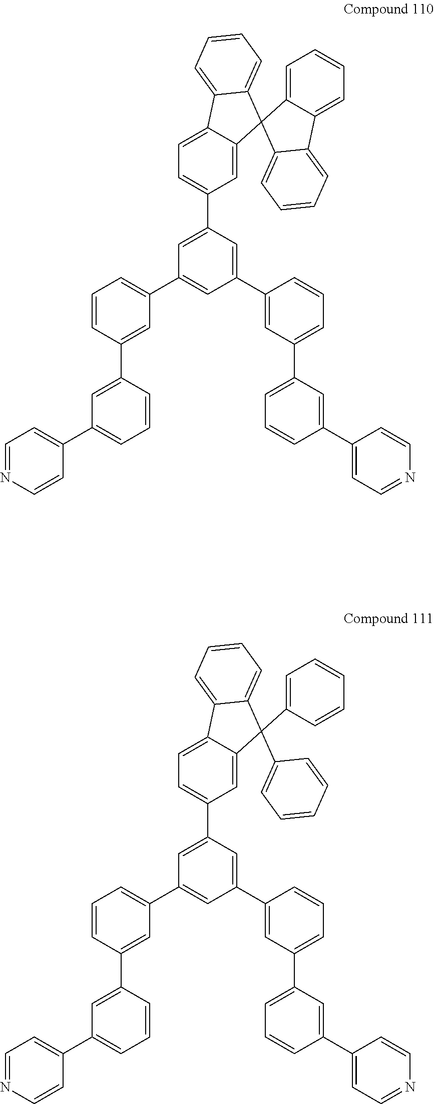

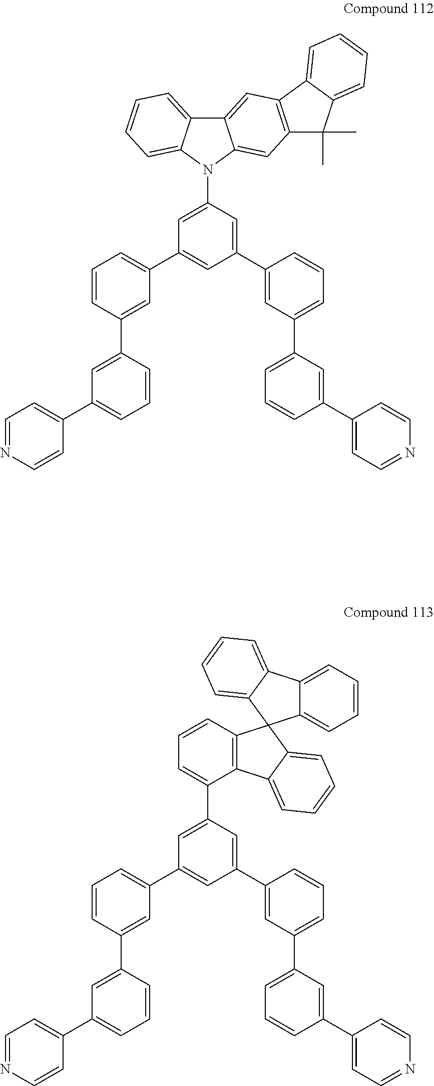

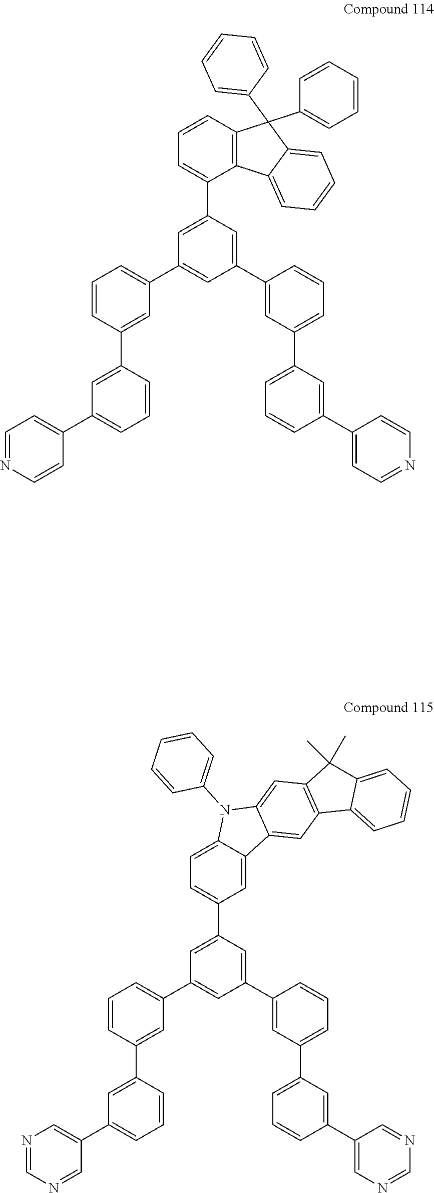

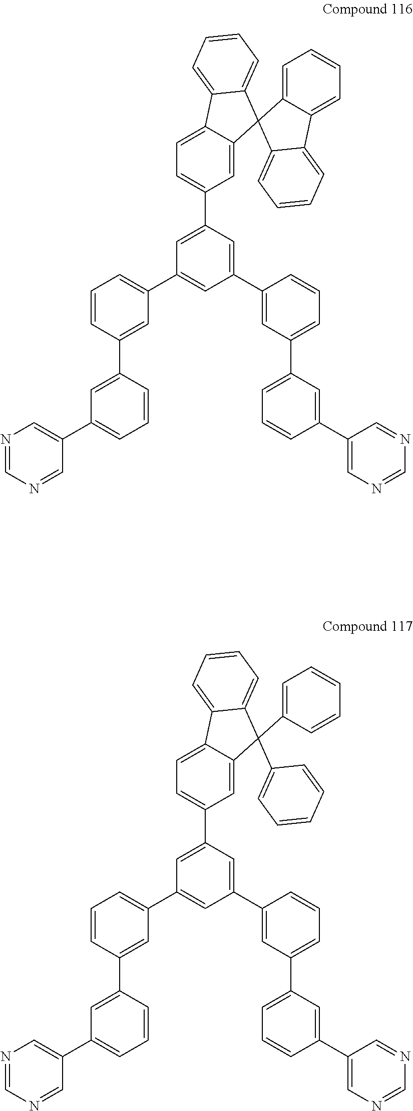

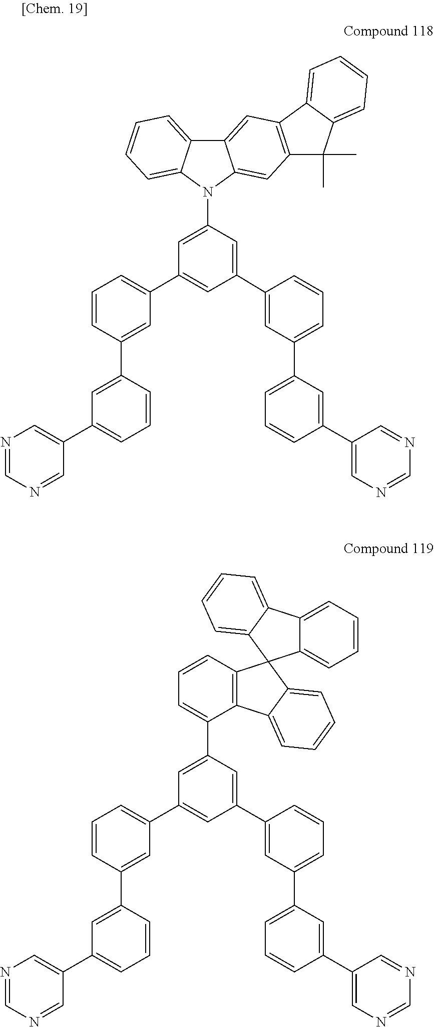

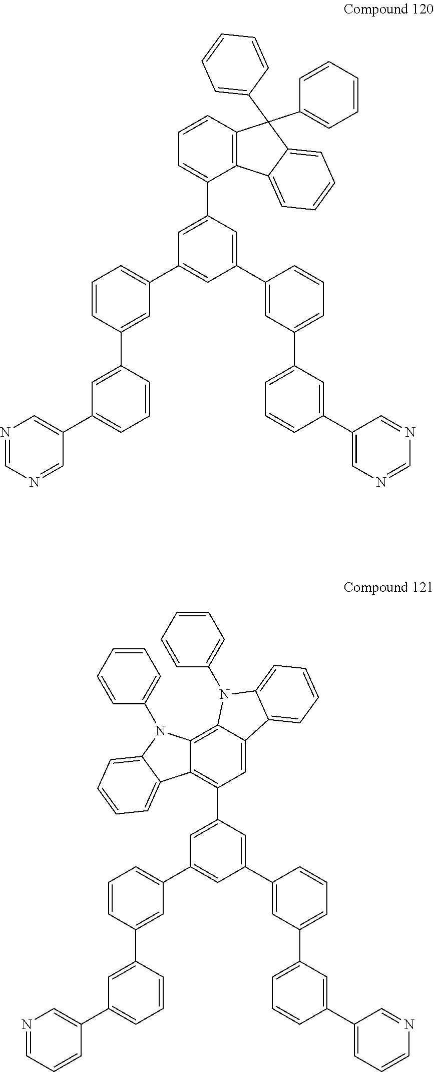

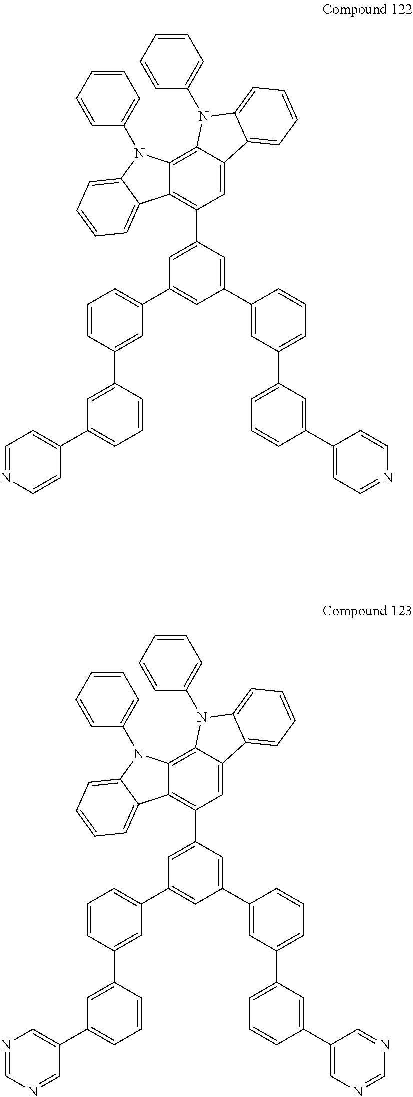

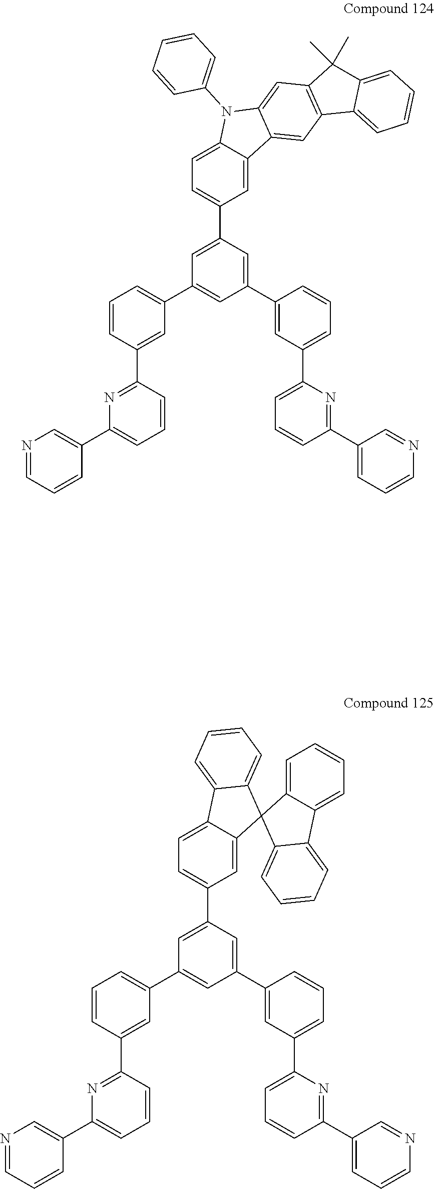

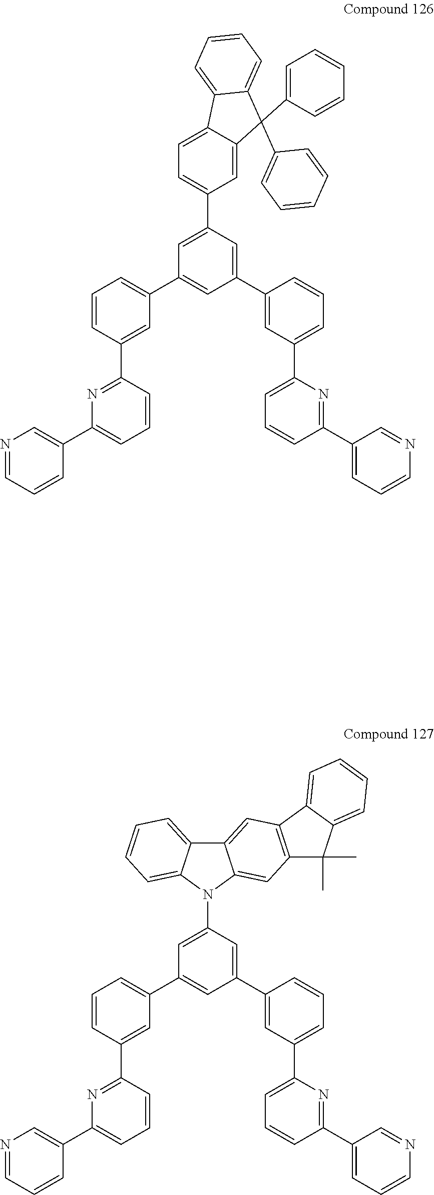

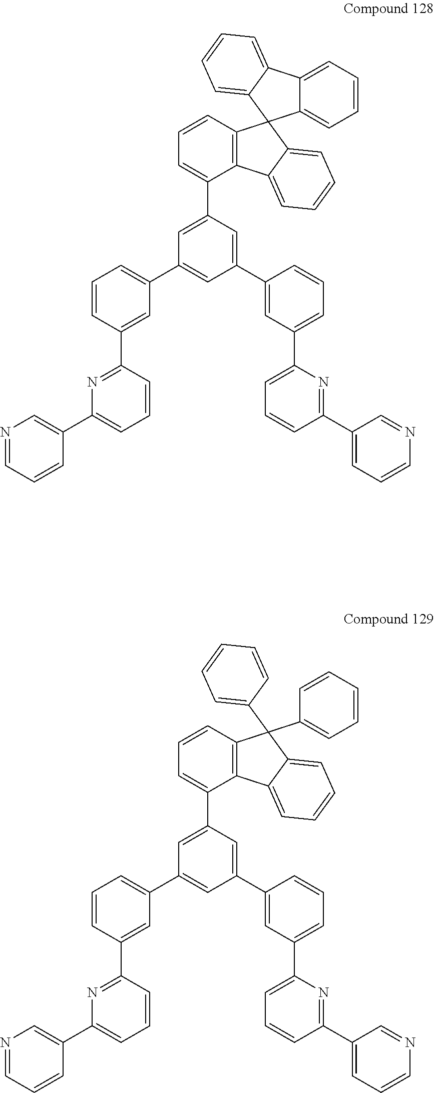

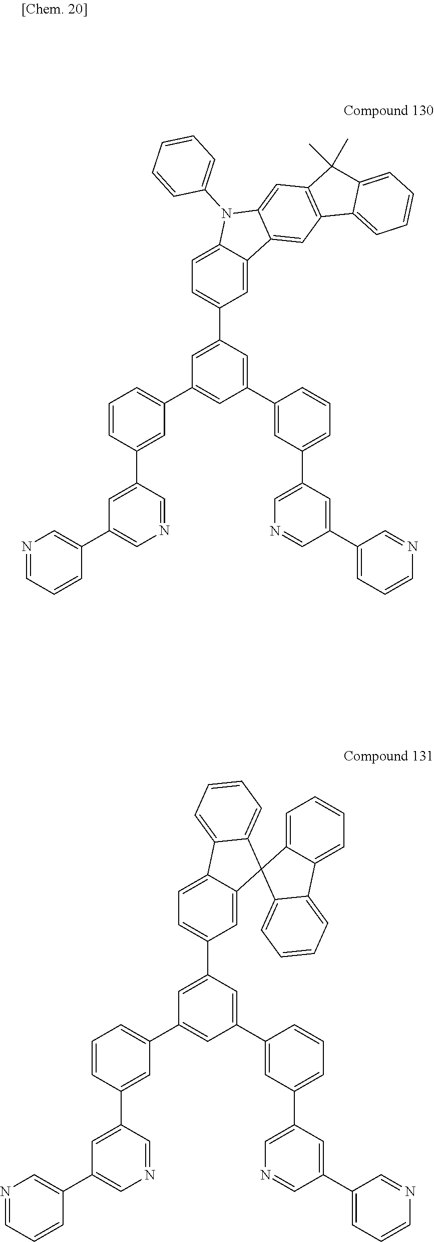

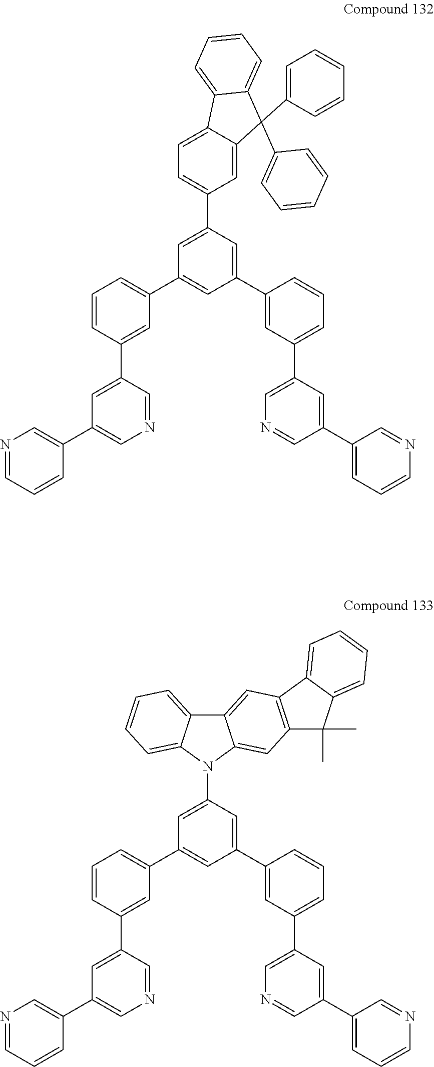

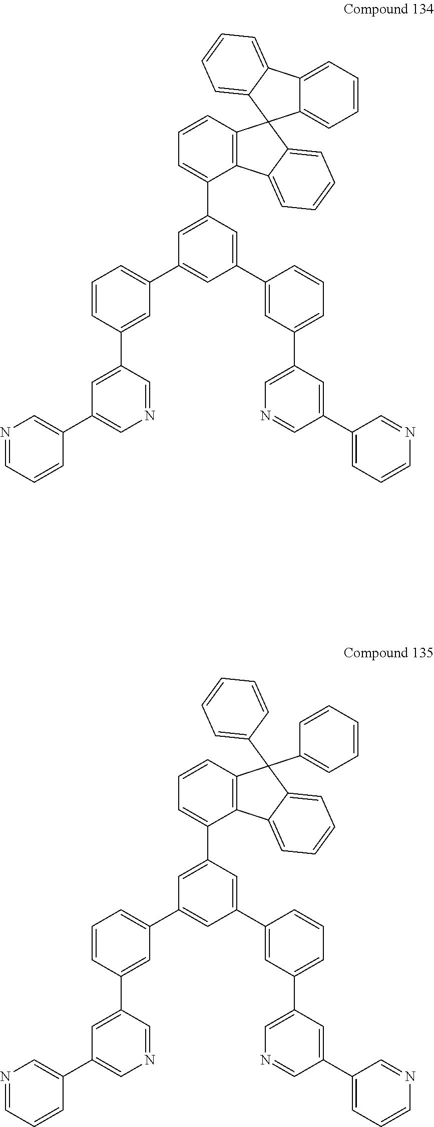

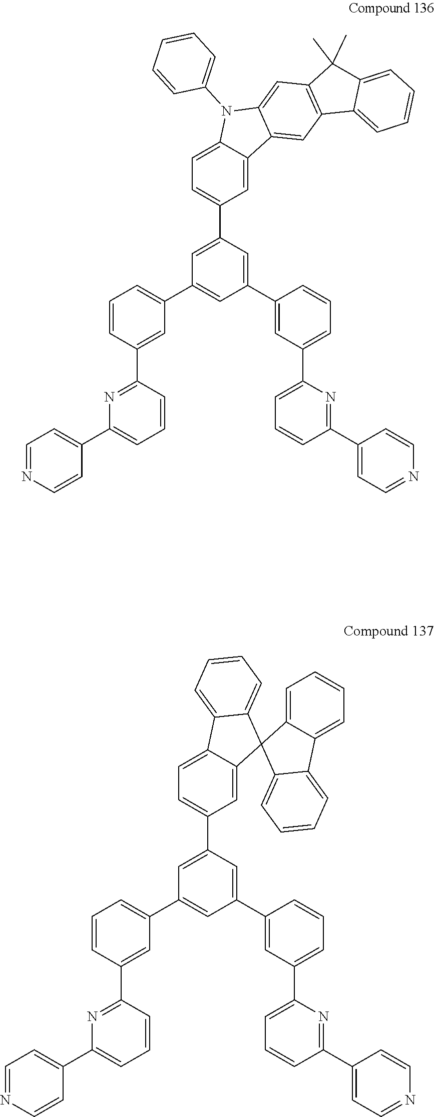

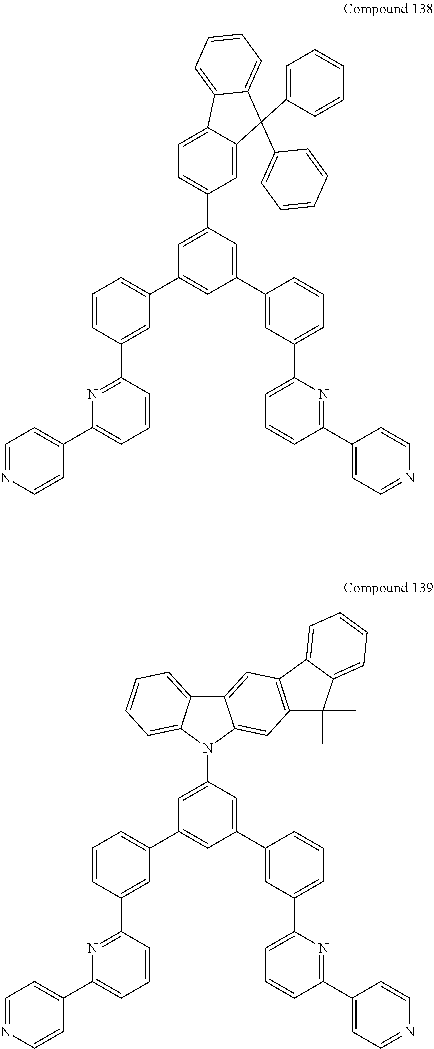

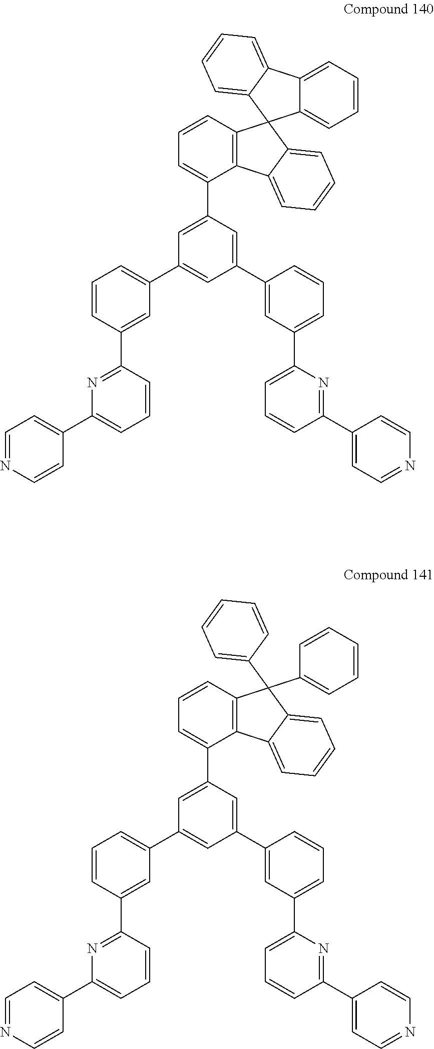

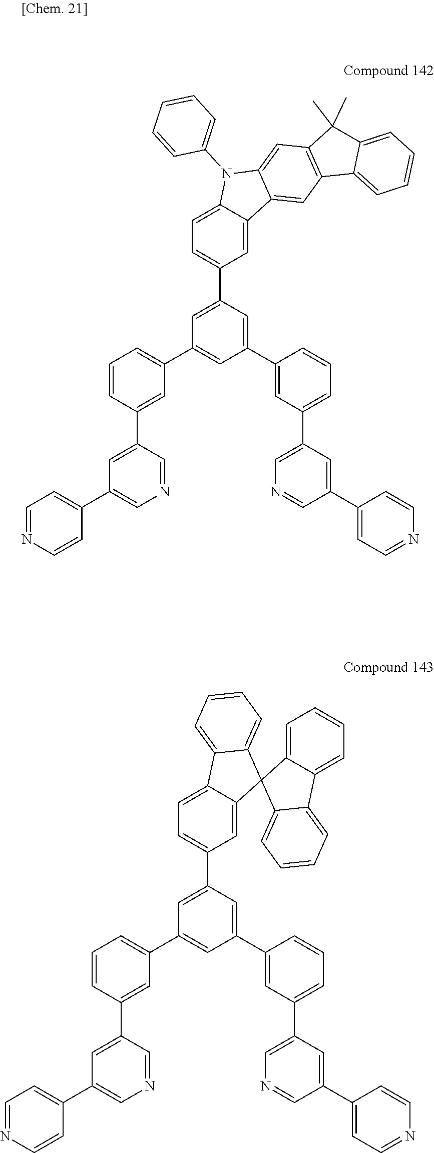

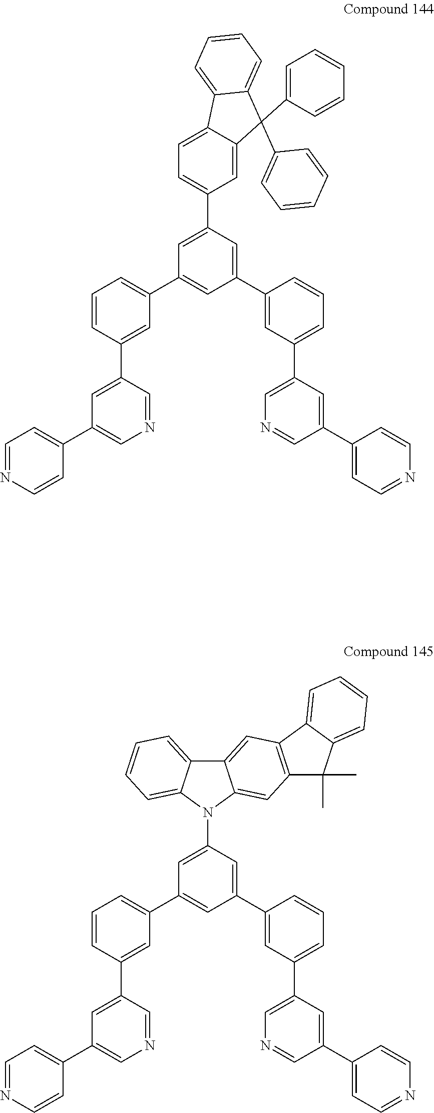

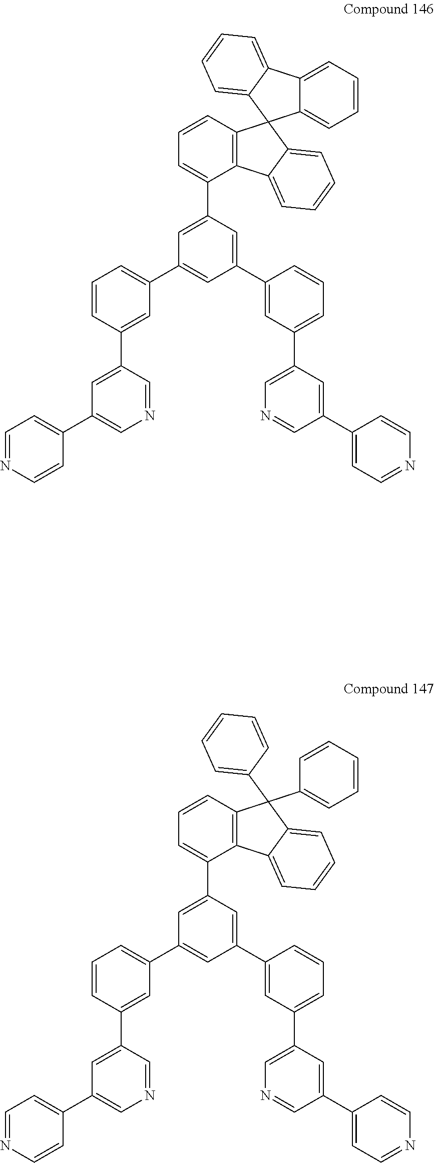

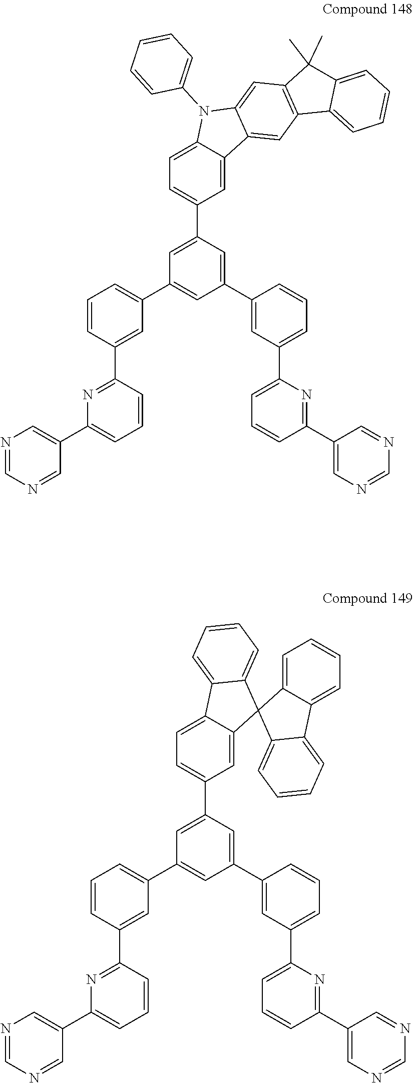

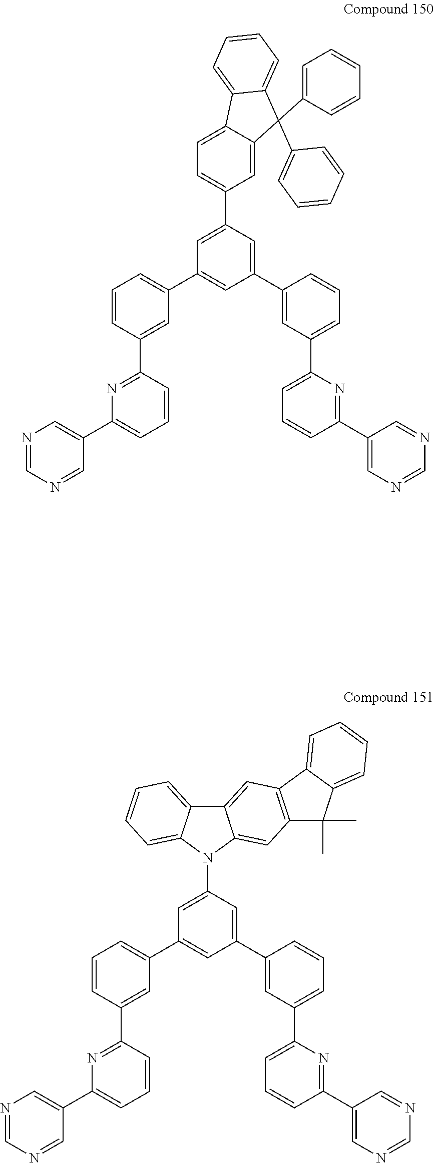

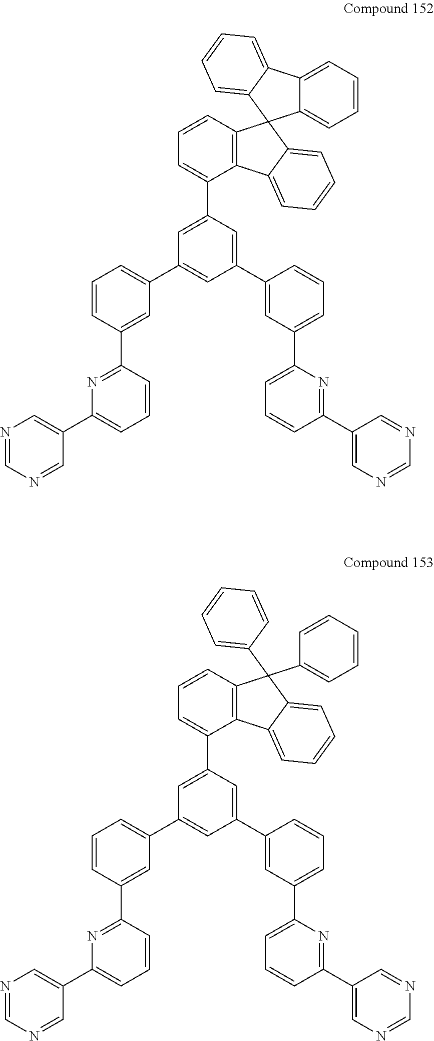

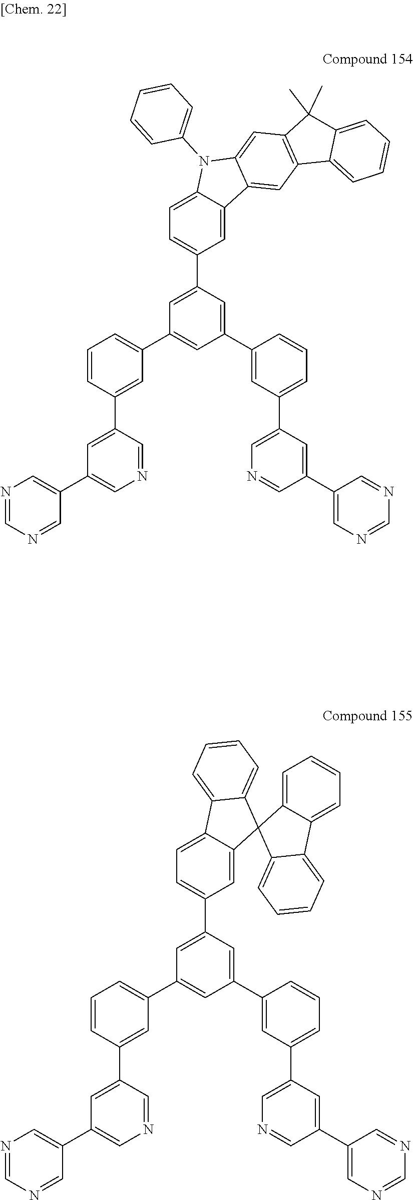

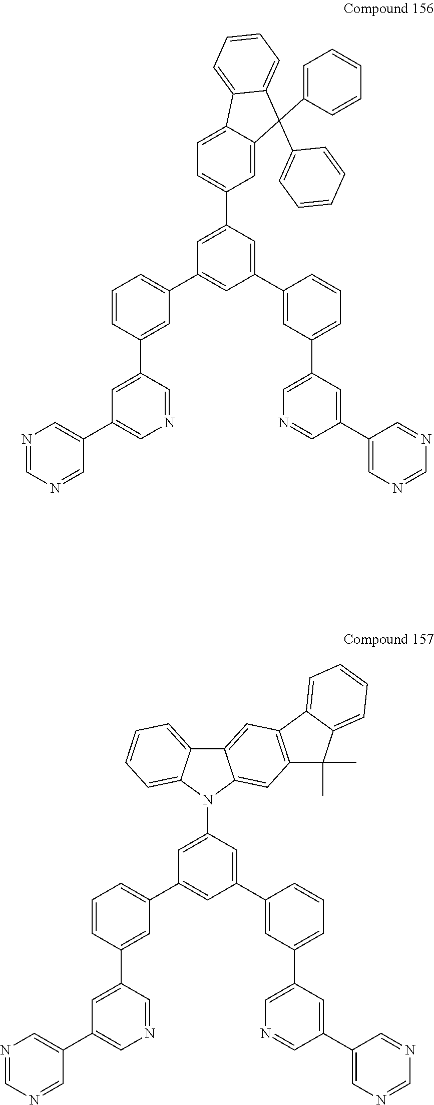

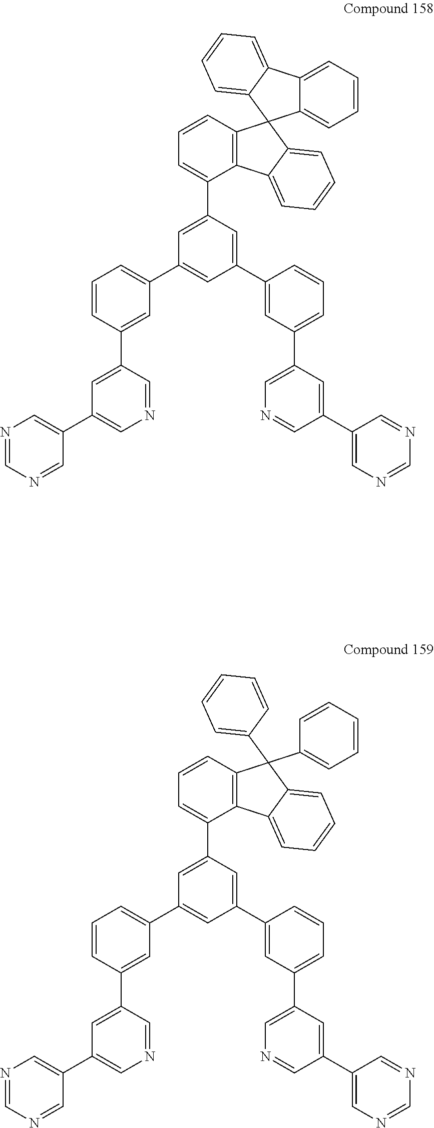

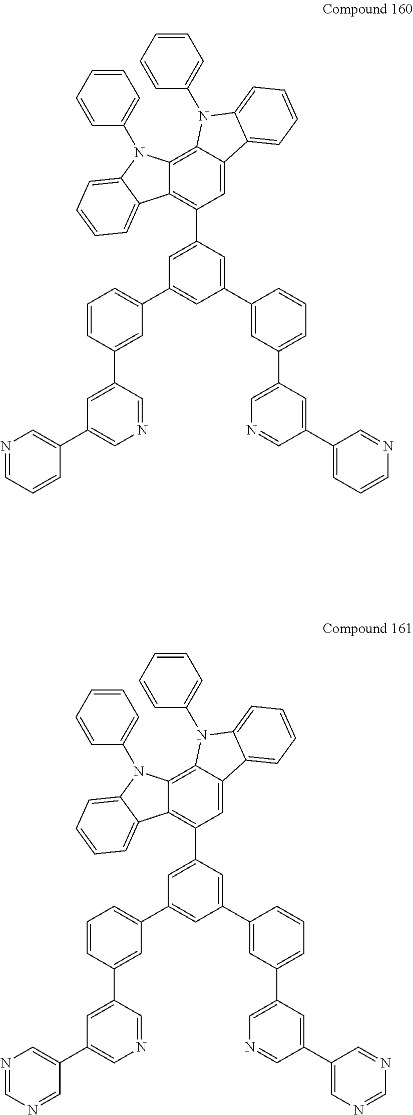

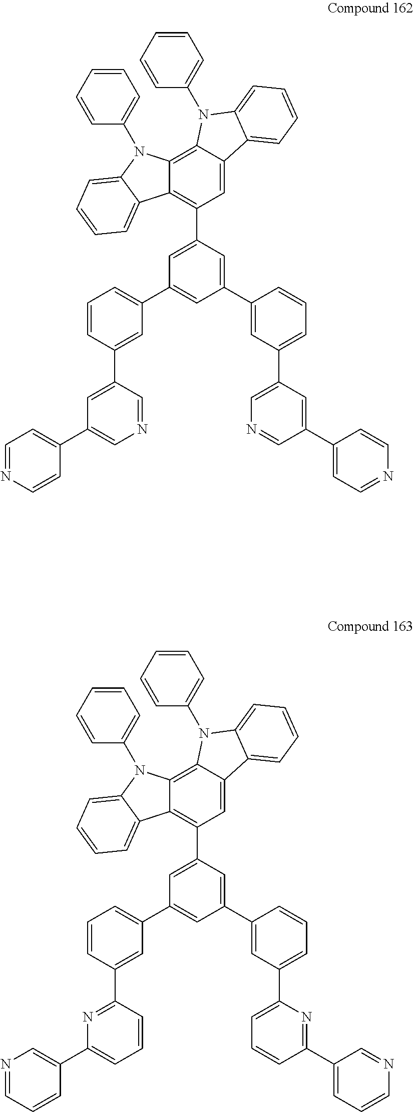

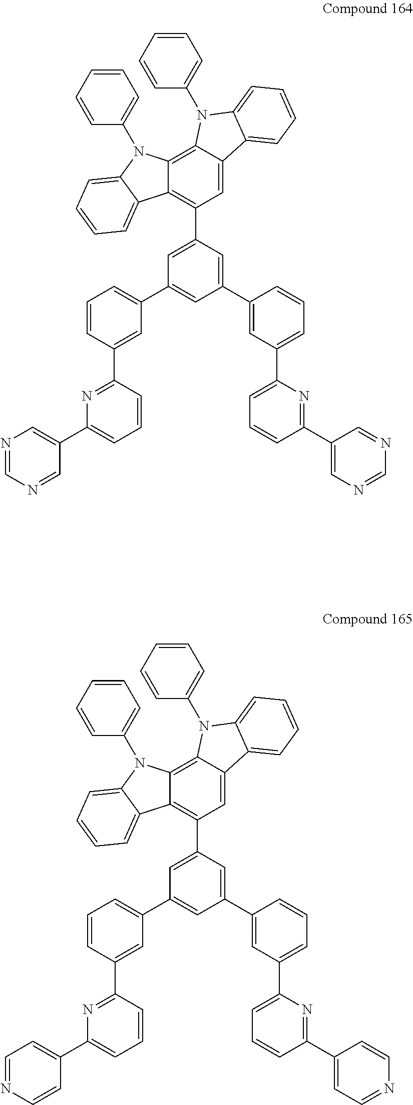

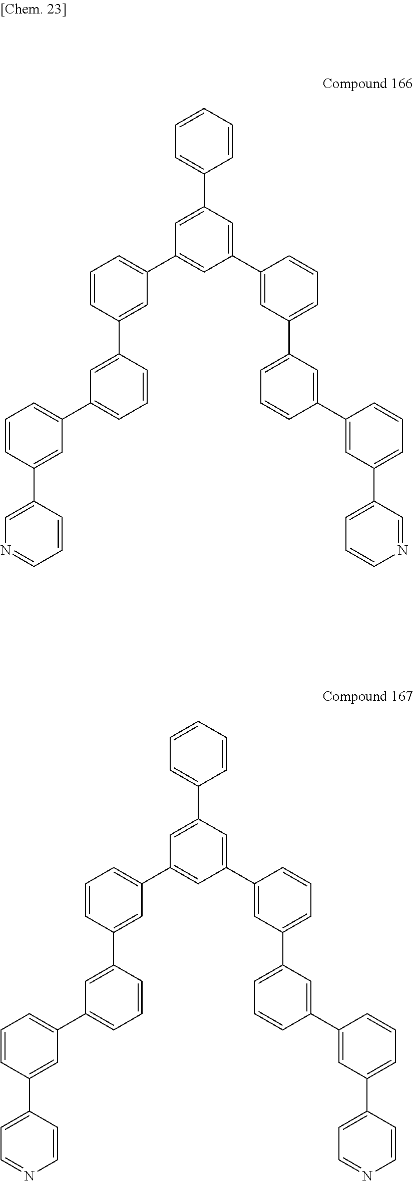

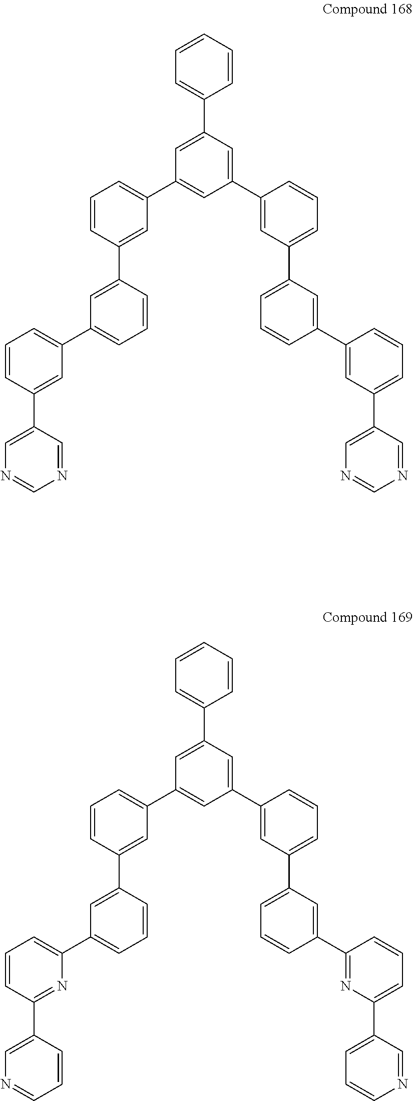

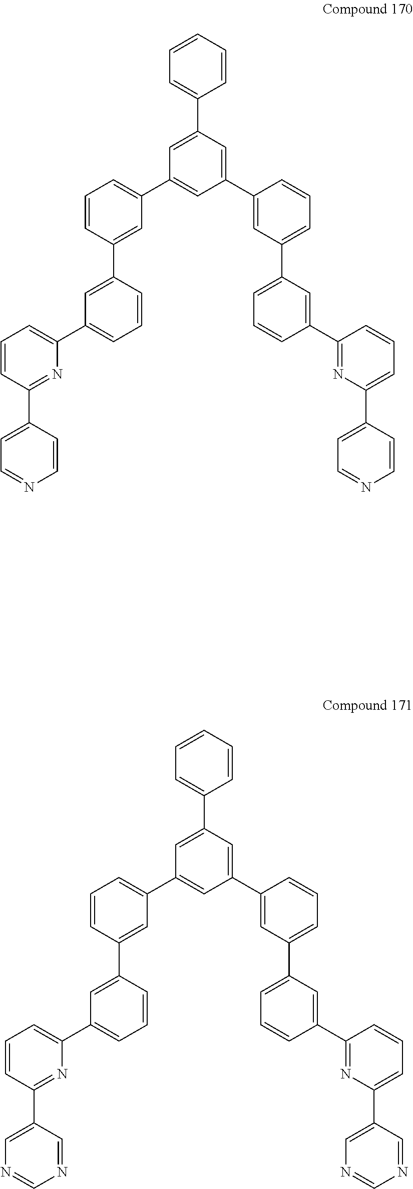

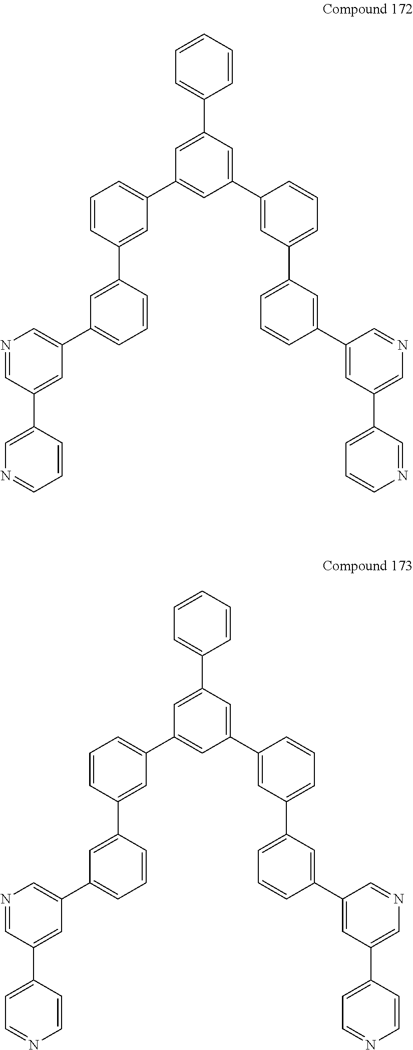

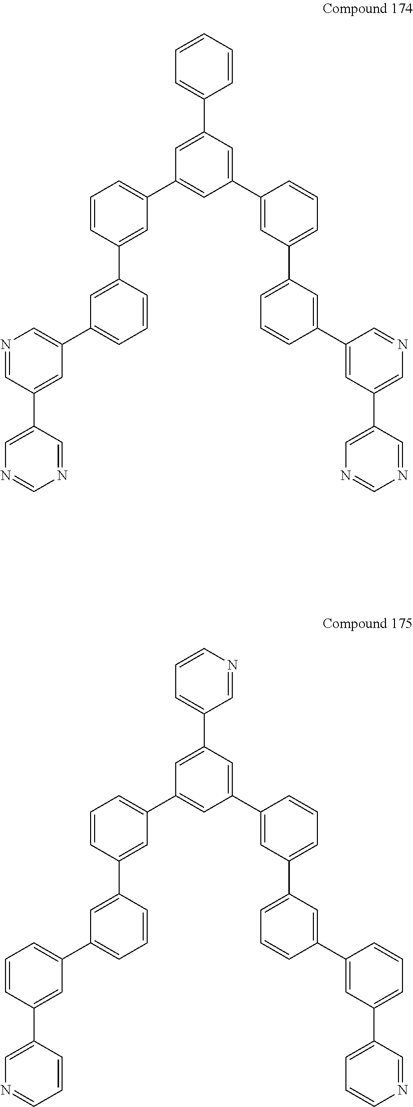

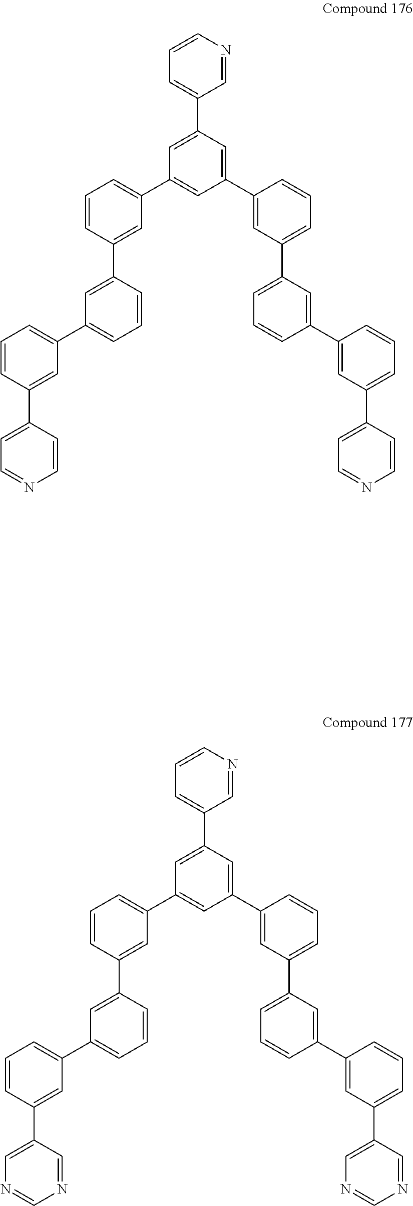

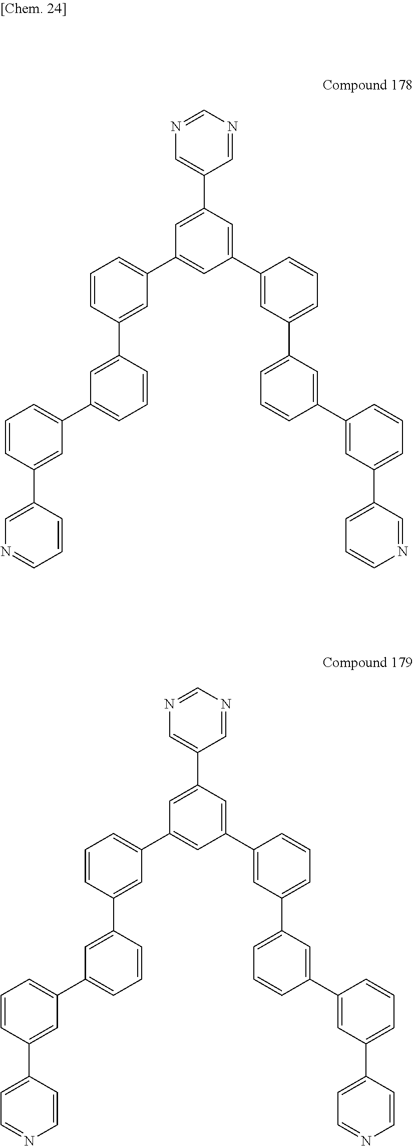

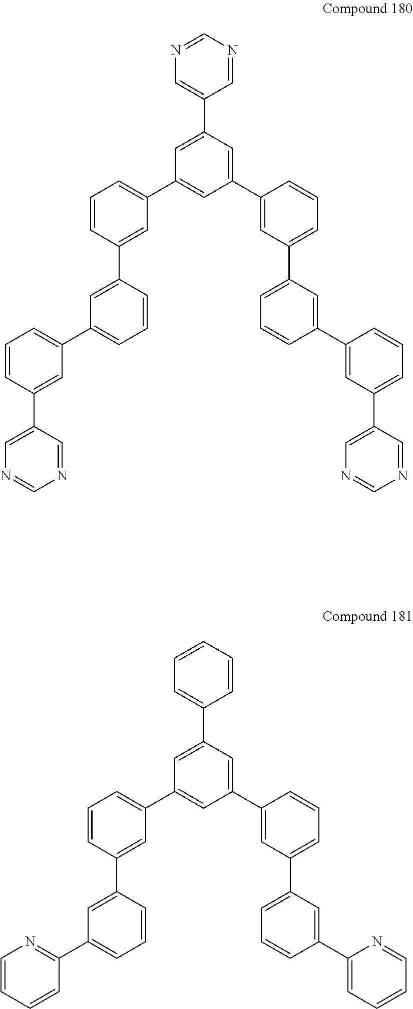

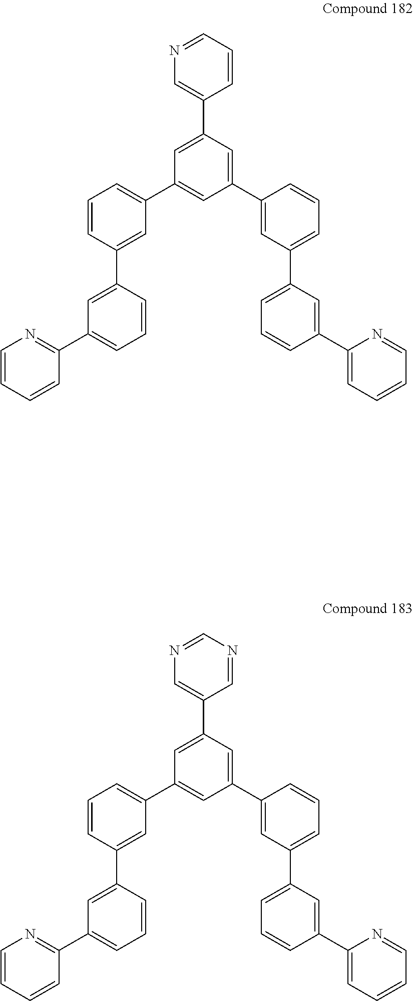

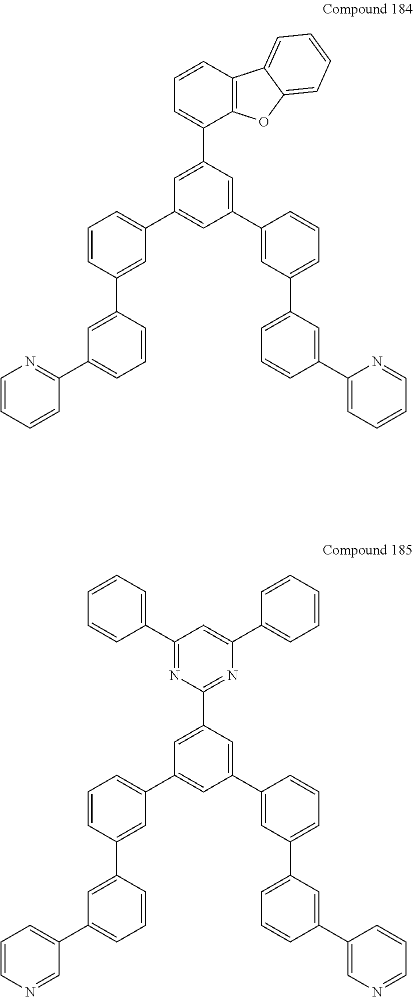

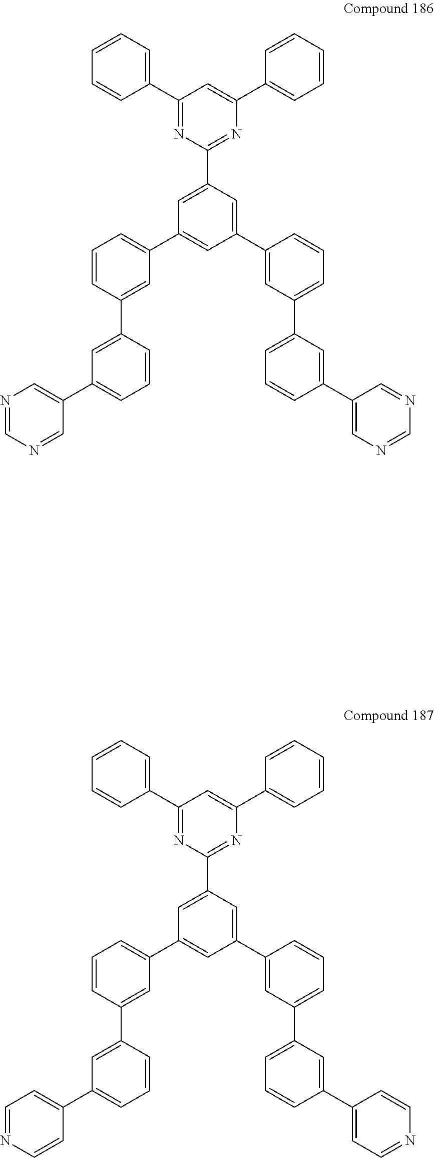

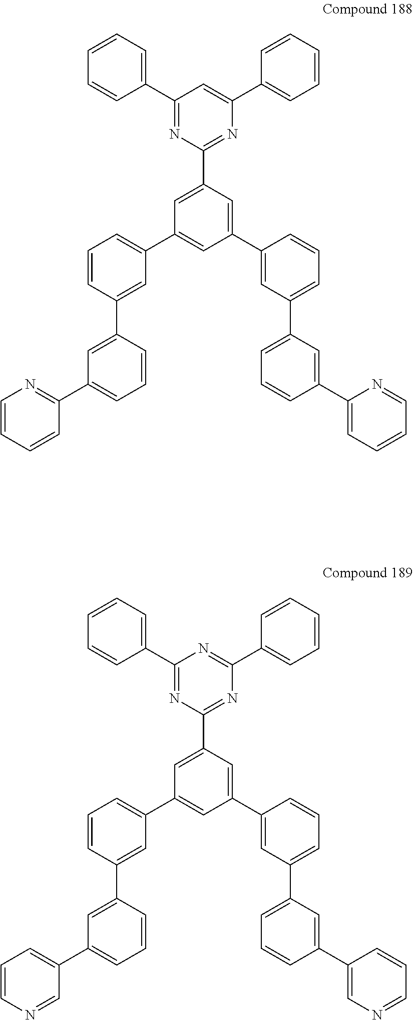

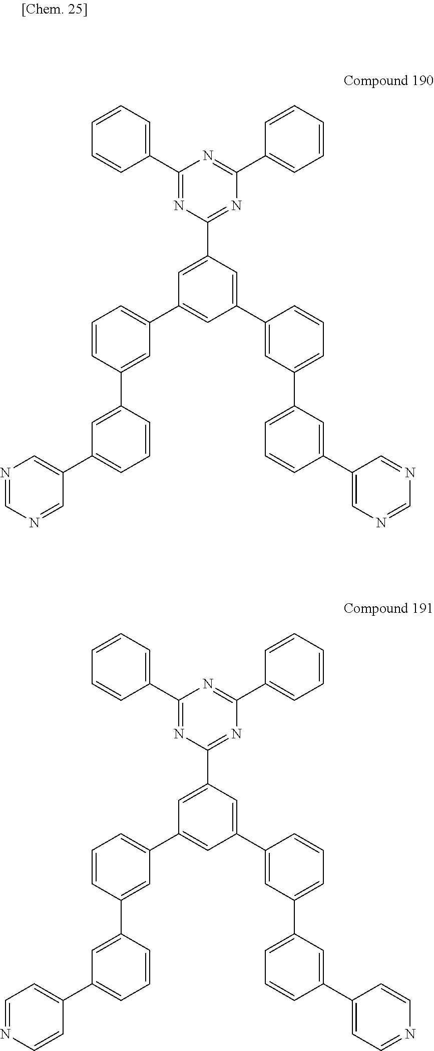

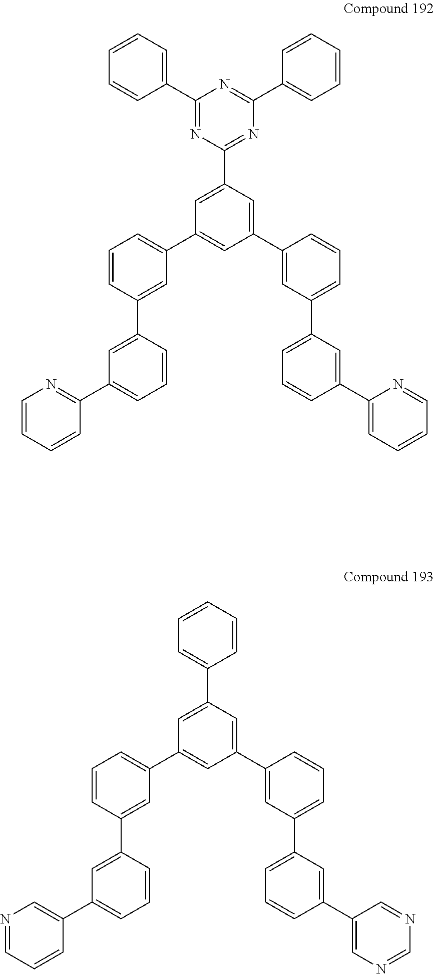

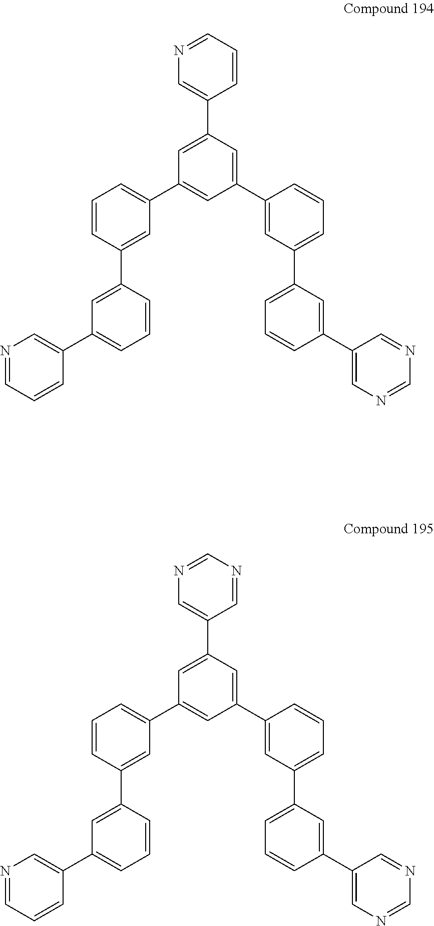

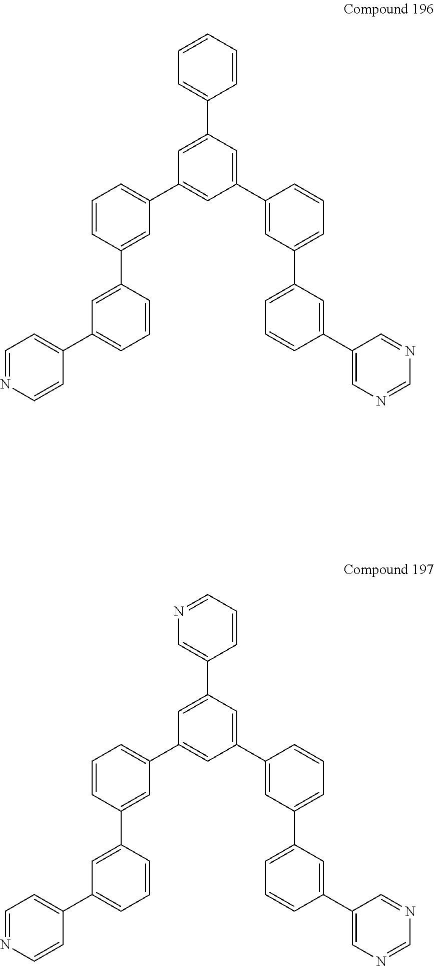

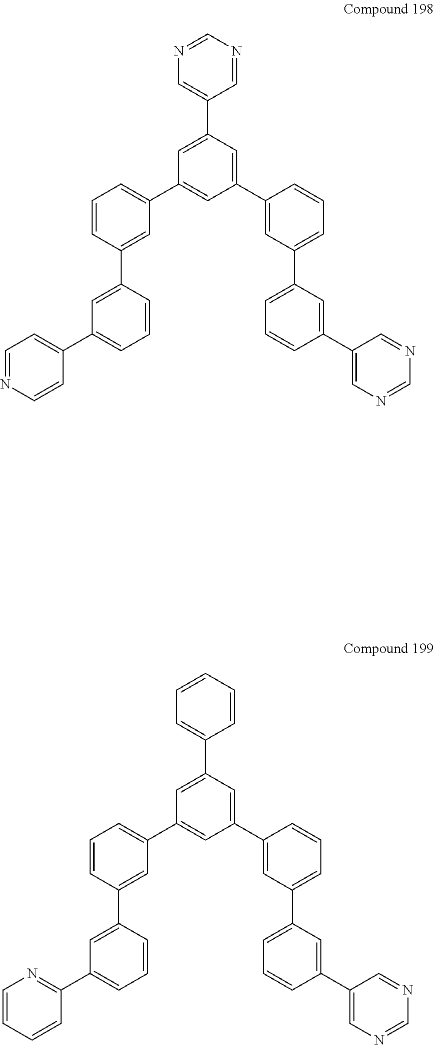

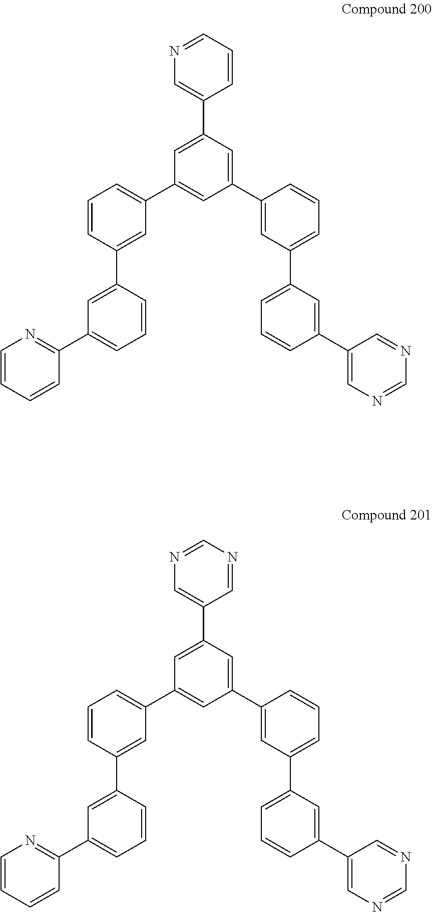

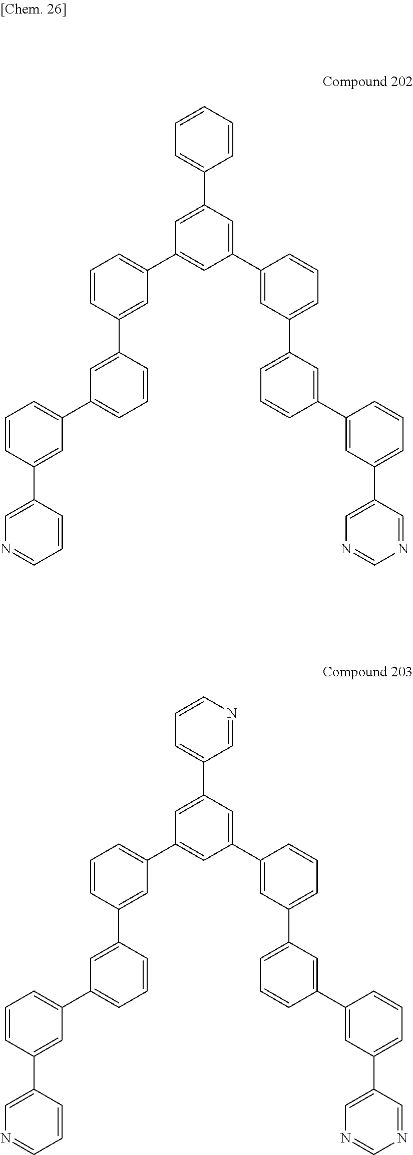

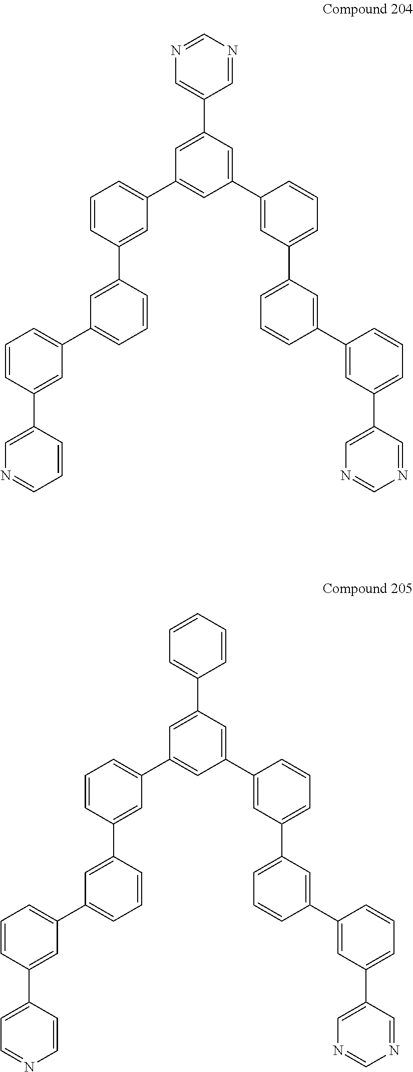

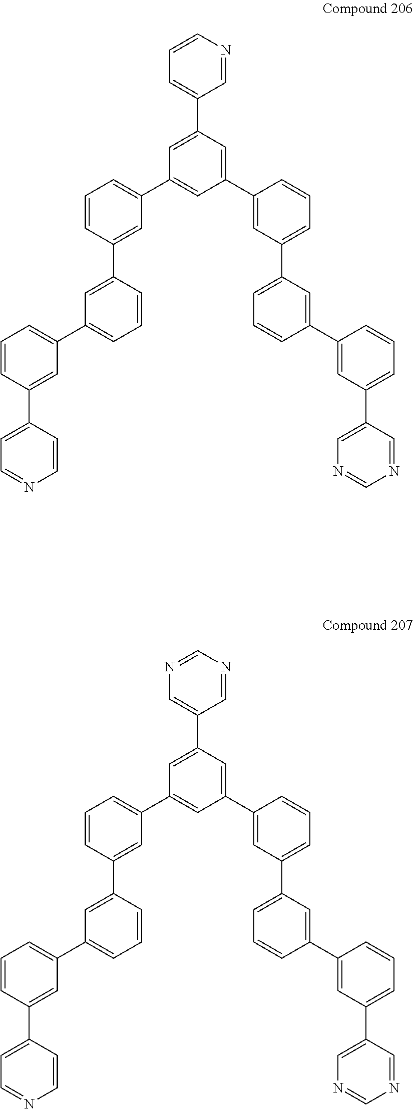

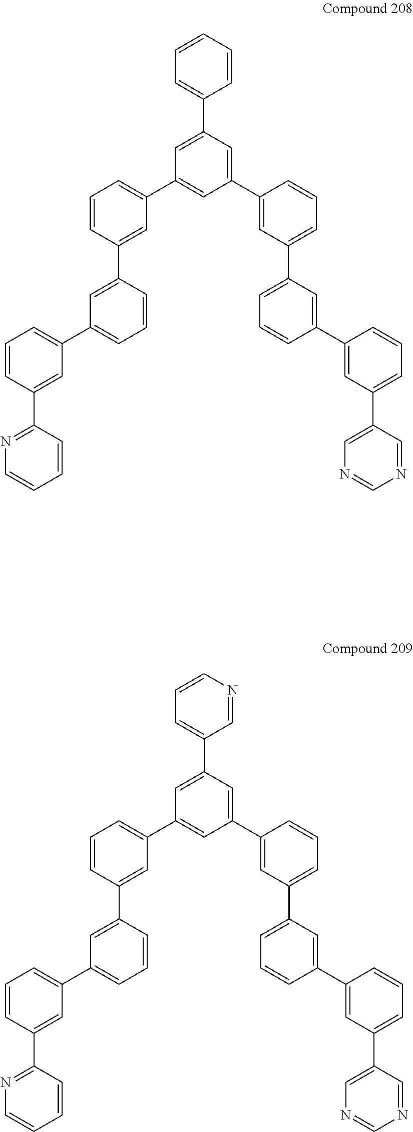

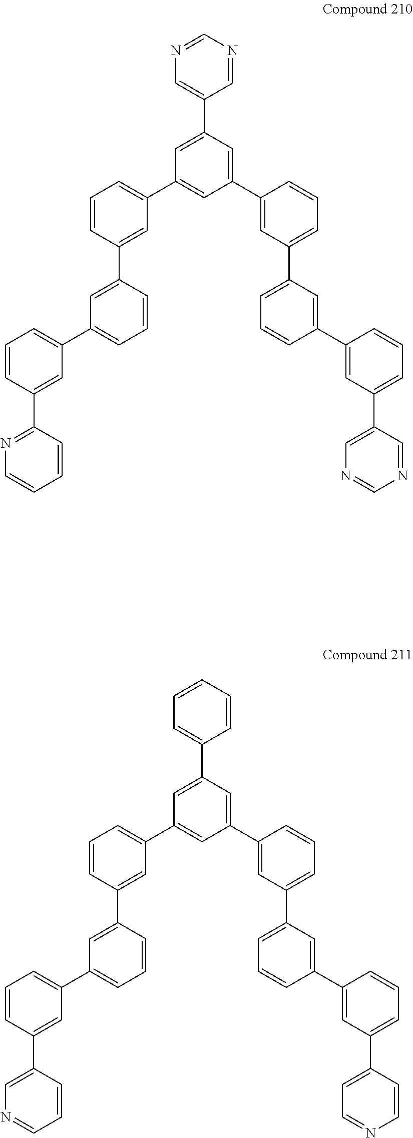

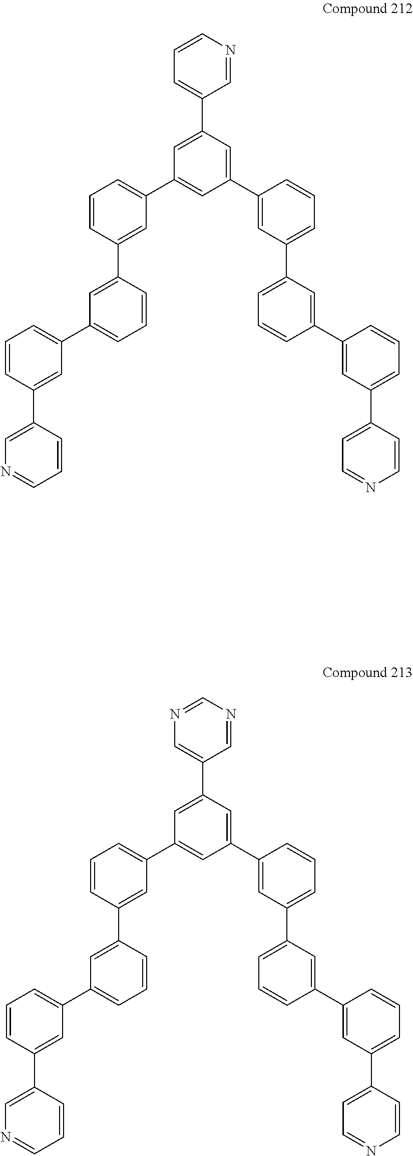

The purification of these compounds is performed by purification through column chromatography, adsorption purification by silica gel, activated carbon, activated clay, or the like, recrystallization or a crystallization method from a solvent, or the like. The compound is identified by NMR analysis. As physical property values, a melting point, a glass transition point (Tg), a band gap, and a work function are measured. The melting point serves as an index of the vapor deposition property, the glass transition point (Tg) serves as an index of the stability of the thin film state, the work function serves as an index of the hole blocking ability, and the band gap is a parameter for calculating the electron affinity.

The melting point and the glass transition point are measured by using a powder and using a high-sensitivity differential scanning calorimeter DSC6200 manufactured by Seiko Instruments Inc. The glass transition point of the compounds represented by the general formulas (1) to (6) is not particularly limited, but is preferably 80° C. or higher from the viewpoint of the stability of the formed thin film. The upper limit of the glass transition point is not particularly limited, and for example, a compound having a glass transition temperature of 250° C. or lower can be used.

The work function is measured by preparing a thin film having a thickness of 100 nm on an ITO substrate, and using an atmospheric photoelectron spectrometer AC-3 manufactured by Riken Keiki Co., Ltd. The work function of the vapor-deposited film having a film thickness of 100 nm formed on the ITO substrate by using the compound represented by the general formulas (1) to (6) is not particularly limited, and is preferably larger than 6.3 eV. The upper limit of the work function of the vapor-deposited film is not particularly limited, and, for example, a vapor-deposited film having a work function of 7.0 eV or less can be used.

The band gap can be calculated from an ultraviolet-visible absorption spectrum measured by a commercially available spectrophotometer. The band gap can be calculated by reading the wavelength of the absorption edge on the long wavelength side and converting it into an energy value of light according to the following equation.

Eg(eV)=hc/λ,

Here, Eg represents a value of the band gap converted into light energy, h represents a Planck constant (6.63×10⁻³⁴ Js), c represents a speed of light (3.00×10⁸ m/s), and λ represents a wavelength (nm) on the long wavelength side absorption edge of the ultraviolet-visible absorption spectrum. Then, 1 eV is 1.60×10⁻¹⁹ J.

In general, the electron affinity (Ea) can be calculated based on the work function (Ip) and the band gap (Eg) according to the following equation.

Ea(eV)=Ip−Eg

The electron affinity (Ea) of the compound represented by the general formulas (1) to (6) calculated based on the work function of the vapor-deposited film having a film thickness of 100 nm formed on the ITO substrate by using the compound represented by the general formulas (1) to (6) and the band gap (Eg) calculated from the above equation is not particularly limited, but is preferably smaller than 2.80 eV. The lower limit of the electron affinity (Ea) is not particularly limited, and for example, a vapor-deposited film having an electron affinity of 2.0 eV or more can be used.

Examples of a structure of the organic EL device of the present embodiment include a structure including an anode, a hole transport layer, a light emitting layer, a hole blocking layer, an electron transport layer, and a cathode in this order on a substrate, a structure further including a hole injection layer between the anode and the hole transport layer, a structure including an electron injection layer between the electron transport layer and the cathode, and a structure including an electron blocking layer between the light emitting layer and the hole transport layer. In these multilayer structures, some of organic layers can be omitted, and examples thereof include a configuration including an anode, a hole transport layer, a light emitting layer, an electron transport layer, and a cathode in this order on a substrate.

Each of the light emitting layer, the hole transport layer, and the electron transport layer may have a structure in which two or more layers are laminated.

As the anode of the organic EL device of the present embodiment, an electrode material having a large work function such as ITO or gold is used. As the hole injection layer of the organic EL device of the present embodiment, in addition to a porphyrin compound represented by copper phthalocyanine, use can be made of a triphenylamine trimer or tetramer such as a starburst type triphenylamine derivative and an arylamine compound having a structure in which three or more triphenylamine structures are linked by a single bond or a divalent group containing no hetero atom in a molecule, an acceptor heterocyclic compound such as hexacyanoazatriphenylene, and a coating type polymer material. These materials can form a thin film by a publicly known method such as a spin coating method or an inkjet method in addition to a vapor deposition method.

As the hole transport layer of the organic EL device of the present embodiment, use can be made of benzidine derivatives such as N,N′-diphenyl-N,N′-di(m-tolyl)-benzidine (hereinafter, abbreviated as TPD), N,N′-diphenyl-N,N′-di(α-naphthyl)-benzidine (hereinafter, abbreviated as NPD), and N,N,N′,N′-tetrabiphenylyl benzidine, 1,1-bis[(di-4-tolylamino) phenyl] cyclohexane (hereinafter, abbreviated as TAPC), various triphenylamine trimers and tetramers, and the like. These may be used to form a film alone or may be used as a single layer formed by being mixed with another material, or may be used as a laminated structure of layers each formed alone, layers each formed by being mixed with another material, or a layer formed alone and a layer formed by being mixed with another material. As a hole injection and transport layer, a coating type polymer material such as poly(3,4-ethylene dioxythiophene) (hereinafter, abbreviated as PEDOT)/poly(styrene sulfonate) (hereinafter abbreviated as PSS) can be used. These materials can form a thin film by a publicly known method such as a spin coating method or an inkjet method in addition to a vapor deposition method.

In the hole injection layer or the hole transport layer, a material that is obtained by further P-doping a material generally used for the layer with trisbromophenylamine hexachloroantimony or the like, a polymer compound having a TPD structure in a partial structure thereof, or the like can be used.

As the electron blocking layer of the organic EL device of the present embodiment, use can be made of a compound having an electron blocking effect such as a carbazole derivative such as 4,4′,4″-tri(N-carbazolyl) triphenylamine (hereinafter, abbreviated as TCTA), 9,9-bis[4-(carbazole-9-yl) phenyl] fluorene, 1,3-bis(carbazole-9-yl) benzene (hereinafter, abbreviated as mCP), or 2,2-bis(4-carbazole-9-ylphenyl) adamantane (hereinafter, abbreviated as Ad-Cz), and a compound having a triphenylsilyl group and a triarylamine structure, which is represented by 9-[4-(carbazole-9-yl) phenyl]-9-[4-(triphenylsilyl) phenyl]-9H-fluorene. These may be used to form a film alone or may be used as a single layer formed by being mixed with another material, or may be used as a laminated structure of layers each formed alone, layers each formed by being mixed with another material, or a layer formed alone and a layer formed by being mixed with another material. These materials can form a thin film by a publicly known method such as a spin coating method or an inkjet method in addition to a vapor deposition method.

As the light emitting layer of the organic EL device of the present embodiment, in addition to the compound represented by the general formulas (1) to (6) of the present embodiment, use can be made of a metal complex of a quinolinol derivative such as Alq₃, various metal complexes, an anthracene derivative, a bisstyrylbenzene derivative, a pyrene derivative, an oxazole derivative, a polyparaphenylene vinylene derivative, and the like. The light emitting layer may be configured with a host material and a dopant material, and in addition to the above-described light emitting material, a thiazole derivative, a benzimidazole derivative, a polydialkylfluorene derivative, or the like can be used as the host material. As the dopant material, quinacridone, coumarin, rubrene, perylene and derivatives thereof, a benzopyran derivative, a rhodamine derivative, an aminostyryl derivative and the like can be used. These may be used to form a film alone or may be used as a single layer formed by being mixed with another material, or may be used as a laminated structure of layers each formed alone, layers each formed by being mixed with another material, or a layer formed alone and a layer formed by being mixed with another material.

As the light emitting material, a phosphorescent light emitting material can also be used. As the phosphorescent light emitting material, a phosphorescent material of a metal complex such as iridium or platinum can be used. A green phosphorescent material such as Ir(ppy)₃, a blue phosphorescent material such as FIrpic and FIr6, a red phosphorescent material such as Btp₂Ir(acac) and the like are used. As the host material at this time, a carbazole derivative such as 4,4′-di(N-carbazolyl) biphenyl (hereinafter, abbreviated as CBP), TCTA, and mCP can be used as a host material having hole injection and transport properties. As the host material having electron transportability, p-bis(triphenylsilyl) benzene (hereinafter, abbreviated as UGH2), 2,2′,2″-(1,3,5-phenylene)-tris(1-phenyl-1H-benzimidazole) (TPBi), and the like can be used.

In order to avoid concentration quenching, doping of the phosphorescent emitting material into the host material is preferably performed by co-deposition in a range of 1 to 30 weight percents with respect to the entire light emitting layer.

As the light emitting material, a material that emits delayed fluorescence, such as PIC-TRZ, CC2TA, PXZ-TRZ, or 4CzIPN, can also be used. (see, e.g., Non-Patent Literatures 3 and 4)

These materials can form a thin film by a publicly known method such as a spin coating method or an inkjet method in addition to a vapor deposition method.

As the hole blocking layer of the organic EL device of the present embodiment, in addition to the compound represented by the general formulas (1) to (6) of the present embodiment, use can be made of a compound having a hole blocking effect such as a phenanthroline derivative such as bathocuproine (hereinafter, abbreviated as BCP), a metal complex of a quinolinol derivative such as BAlq, various rare earth complexes, an oxazole derivative, a triazole derivative, and a triazine derivative. These materials may also double as the material of the electron transport layer. These may be used to form a film alone or may be used as a single layer formed by being mixed with another material, or may be used as a laminated structure of layers each formed alone, layers each formed by being mixed with another material, or a layer formed alone and a layer formed by being mixed with another material. These materials can form a thin film by a publicly known method such as a spin coating method or an ink jet method in addition to a vapor deposition method.

As the electron transport layer of the organic EL device of the present embodiment, in addition to the compound represented by the general formulas (1) to (6) of the present embodiment, use can be made of various metal complexes, a triazole derivative, a triazine derivative, an oxadiazole derivative, a thiadiazole derivative, a carbodiimide derivative, a quinoxaline derivative, a phenanthroline derivative, a silole derivative, and the like in addition to metal complexes of quinolinol derivatives such as Alq₃ and BAlq. These may be used to form a film alone or may be used as a single layer formed by being mixed with another material, or may be used as a laminated structure of layers each formed alone, layers each formed by being mixed with another material, or a layer formed alone and a layer formed by being mixed with another material. These materials can form a thin film by a publicly known method such as a spin coating method or an inkjet method in addition to a vapor deposition method.

As the electron injection layer of the organic EL device of the present embodiment, in addition to the compound represented by the general formulas (1) to (6) of the present embodiment, use can be made of an alkali metal salt such as lithium fluoride and cesium fluoride, an alkaline earth metal salt such as magnesium fluoride, a metal oxide such as aluminum oxide, and the like, but this can be omitted in a preferred selection of the electron transport layer and the cathode.

Furthermore, in the electron injection layer or the electron transport layer, it is possible to use a material obtained by further N-doping a material generally used in the layer with a metal such as cesium or a metal complex such as lithium quinoline.

As the cathode of the organic EL device of the present embodiment, an electrode material having a small work function, such as aluminum, or an alloy having a smaller work function, such as a magnesium-silver alloy, a magnesium-indium alloy, and an aluminum-magnesium alloy, is used as the electrode material.

The embodiments of the present invention will be described more specifically by Examples below, but the present invention is restricted to the following Examples.

### Example 1

### 3-{m-[m-(5-phenyl-3-{m[m-(3-pyridyl) phenyl] phenyl} phenyl) phenyl] phenyl} pyridine (Compound 1)

To a reaction vessel in which the atmosphere was replaced with nitrogen gas, 28.4 g of 1-bromo-3-iodobenzene, 20.0 g of 3-(3-pyridyl) phenylboronic acid, 160 ml of toluene, 40 ml of ethanol, 75 ml of a 2M potassium carbonate aqueous solution, and 2.3 g of tetrakis (triphenylphosphine) palladium (0) were added, and the mixture was heated to reflux for 8 hours while being stirred. The mixture was cooled to reach room temperature, separated, dehydrated with anhydrous magnesium sulfate, and then concentrated to obtain a crude product. The crude product was purified by column chromatography (carrier: silica gel, eluent: ethyl acetate/n-hexane=1/2) to obtain 34.8 g (yield: 89%) of 3-(3′-bromo-3-biphenylyl) pyridine as a white powder.

To a reaction vessel in which the atmosphere was replaced with nitrogen gas, 30.5 g of 3-(3′-bromo-3-biphenylyl) pyridine, 30.0 g of bis(pinacolato) diboron, 14.5 g of potassium acetate, 305 ml of 1,4-dioxane, and 1.6 g of a [1,1′-bis(diphenylphosphino) ferrocene] dichloropalladium (II) dichloromethane adduct were added, and the mixture was heated at 100° C. for 4 hours while being stirred. The mixture was cooled to reach room temperature, separated by adding 500 ml of toluene and saturated saline solution, dehydrated with anhydrous magnesium sulfate, and then concentrated to obtain a crude product. The crude product was purified by column chromatography (carrier: silica gel, eluent: ethyl acetate/n-hexane=1/1) to obtain 30.1 g (yield: 75%) of 3-[3′-(4,4,5,5-tetramethyl-1,3,2-dioxaborolan-2-yl) biphenyl-3-yl] pyridine as a white powder.

To a reaction vessel in which the atmosphere was replaced with nitrogen gas, 2.1 g of 3,5-dibromobiphenyl, 5.1 g of 3-[3′-(4,4,5,5-tetramethyl-1,3,2-dioxaborolan-2-yl) biphenyl-3-yl] pyridine, 40 ml of toluene, 10 ml of ethanol, 10 ml of a 2M potassium carbonate aqueous solution, and 0.16 g of tetrakis(triphenylphosphine) palladium (0) were added, and the mixture was heated to reflux for 16 hours while being stirred. The mixture was cooled to reach room temperature, separated, dehydrated with anhydrous magnesium sulfate, and then concentrated to obtain a crude product. The crude product was purified by column chromatography (carrier: silica gel, eluent: ethyl acetate/n-hexane=2/1) to obtain 3.5 g (yield: 85%) of 3-{m-[m-(5-phenyl-3-{m-[m-(3-pyridyl) phenyl] phenyl} phenyl) phenyl] phenyl} pyridine (Compound 1) as a white powder.

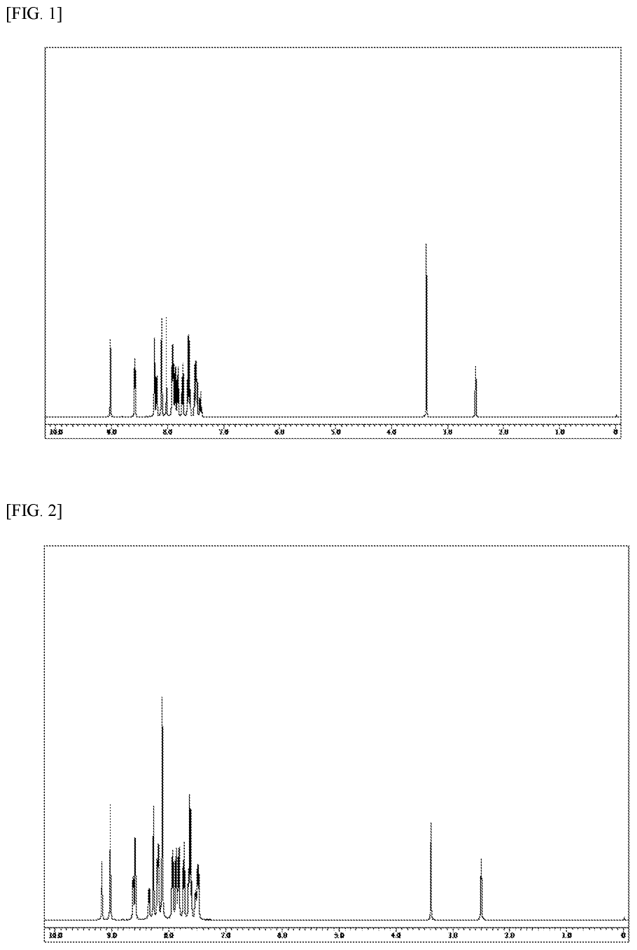

The structure of the obtained white powder was identified by using NMR. The 1H-NMR measurement results are shown in FIG. 1.

The following 32 hydrogen signals were detected by 1H-NMR (DMSO-d6). δ (ppm)=9.02 (2H), 8.58-8.59 (2H), 8.24 (2H), 8.19-8.21 (2H), 8.11 (3H), 8.02 (2H), 7.90-7.93 (4H), 7.86-7.88 (2H), 7.81-7.83 (2H), 7.73-7.65 (2H), 7.60-7.65 (4H), 7.47-7.53 (4H), 7.39-7.43 (1H).

### Example 2

### Synthesis of 3-(3,5-bis{m-[m-(3-pyridyl) phenyl] phenyl} phenyl) pyridine (Compound 4)

To a reaction vessel in which the atmosphere was replaced with nitrogen gas, 5.1 g of 1,3-dibromo-5-iodobenzene, 1.7 g of 3-pyridineboronic acid, 40 ml of toluene, 10 ml of ethanol, 11 ml of a 2M potassium carbonate aqueous solution, and 0.33 g of tetrakis(triphenylphosphine) palladium (0) were added, and the mixture was heated to reflux for 19 hours while being stirred. The mixture was cooled to reach room temperature, separated, dehydrated with anhydrous magnesium sulfate, and then concentrated to obtain a crude product. The crude product was purified by column chromatography (carrier: silica gel, eluent: chloroform) to obtain 2.3 g (yield: 53%) of 3-(3,5-dibromophenyl) pyridine as a pale yellow powder.

To a reaction vessel in which the atmosphere was replaced with nitrogen gas, 2.0 g of 3-(3,5-dibromophenyl) pyridine, 4.8 g of 3-[3′-(4,4,5,5-tetramethyl-1,3,2-dioxaborolan-2-yl) biphenyl-3-yl] pyridine, 40 ml of toluene, 10 ml of ethanol, 10 ml of a 2M potassium carbonate aqueous solution, and 0.17 g of tetrakis(triphenylphosphine) palladium (0) were added, and the mixture was heated to reflux for 14 hours while being stirred. The mixture was cooled to reach room temperature, separated, dehydrated with anhydrous magnesium sulfate, and then concentrated to obtain a crude product. The crude product was purified by column chromatography (carrier: silica gel, eluent: ethyl acetate) to obtain 3.7 g (yield: 94%) of 3-(3,5-bis{m-[m-(3-pyridyl) phenyl] phenyl} phenyl) pyridine (Compound 4) as a white powder.

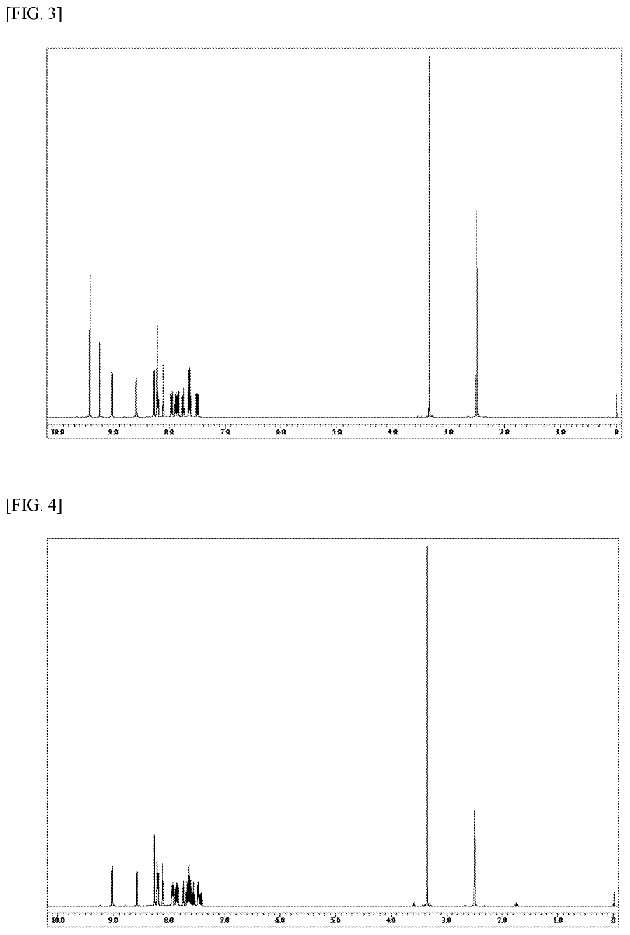

The structure of the obtained white powder was identified by using NMR. The 1H-NMR measurement results are shown in FIG. 2.

The following 31 hydrogen signals were detected by 1H-NMR (DMSO-d6). δ (ppm)=9.18 (1H), 9.03 (2H), 8.62-8.63 (1H), 8.58-8.60 (2H), 8.34-8.36 (1H), 8.27 (2H), 8.18-8.21 (3H), 8.12 (4H), 7.93-7.95 (2H), 7.87-7.88 (2H), 7.82-7.84 (2H), 7.73-7.75 (2H), 7.60-7.65 (4H), 7.47-7.54 (3H).

### Example 3

### Synthesis of 5-(3,5-bis{m-[m-(3-pyridyl) phenyl] phenyl} phenyl) pyrimidine (Compound 7)

To a reaction vessel in which the atmosphere was replaced with nitrogen gas, 5.1 g of 1,3-dibromo-5-iodobenzene, 1.8 g of 5-pyrimidineboronic acid, 40 ml of toluene, 10 ml of ethanol, 11 ml of a 2M potassium carbonate aqueous solution, and 0.33 g of tetrakis(triphenylphosphine) palladium (0) were added, and the mixture was heated to reflux for 23 hours while being stirred. The mixture was cooled to reach room temperature, separated, dehydrated with anhydrous magnesium sulfate, and then concentrated to obtain a crude product. The crude product was purified by column chromatography (carrier: silica gel, eluent: chloroform) to obtain 0.7 g (yield: 16%) of 5-(3,5-dibromophenyl) pyrimidine as a white powder.

To a reaction vessel in which the atmosphere was replaced with nitrogen gas, 0.6 g of 5-(3,5-dibromophenyl) pyrimidine, 1.4 g of 3-[3′-(4,4,5,5-tetramethyl-1,3,2-dioxaborolan-2-yl) biphenyl-3-yl] pyridine, 20 ml of toluene, 5 ml of ethanol, 3 ml of a 2M potassium carbonate aqueous solution, and 50 mg of tetrakis(triphenylphosphine) palladium (0) were added, and the mixture was heated to reflux for 20 hours while being stirred. The mixture was cooled to reach room temperature, separated, dehydrated with anhydrous magnesium sulfate, and then concentrated to obtain a crude product. The crude product was purified by column chromatography (carrier: silica gel, eluent: ethyl acetate) to obtain 0.7 g (yield: 61%) of 5-(3,5-bis{m-[m-(3-pyridyl) phenyl] phenyl} phenyl) pyrimidine (Compound 7) as a white powder.

The structure of the obtained white powder was identified by using NMR. The 1H-NMR measurement results are shown in FIG. 3.

The following 30 hydrogen signals were detected by 1H-NMR (DMSO-d6). δ (ppm)=9.43 (2H), 9.25 (1H), 9.03 (2H), 8.60 (2H), 8.28 (2H), 8.20-8.23 (5H), 8.11 (2H), 7.95-7.97 (2H), 7.88-7.90 (2H), 7.84-7.86 (2H), 7.75-7.77 (2H), 7.62-7.67 (4H), 7.49-7.52 (2H).

### Example 4

### Synthesis of 3-(m-{m-[5-(dibenzofuran-4-yl)-3-{m-[m-(3-pyridyl) phenyl] phenyl} phenyl] phenyl} phenyl) pyridine (Compound 19)

To a reaction vessel in which the atmosphere was replaced with nitrogen gas, 5.0 g of 1,3-dibromo-5-iodobenzene, 2.9 g of dibenzofuran-4-boronic acid, 40 ml of toluene, 10 ml of ethanol, 11 ml of a 2M potassium carbonate aqueous solution, and 0.32 g of tetrakis(triphenylphosphine) palladium (0) were added, and the mixture was heated to reflux for 7 hours while being stirred. The mixture was cooled to reach room temperature, separated, dehydrated with anhydrous magnesium sulfate, and then concentrated to obtain a crude product. The crude product was purified by chloroform/n-hexane crystallization to obtain 2.3 g (yield: 41%) of 4-(3,5-dibromophenyl) dibenzofuran as a white powder.

To a reaction vessel in which the atmosphere was replaced with nitrogen gas, 2.1 g of 4-(3,5-dibromophenyl) dibenzofuran, 3.9 g of 3-[3′-(4,4,5,5-tetramethyl-1,3,2-dioxaborolan-2-yl) biphenyl-3-yl] pyridine, 40 ml of toluene, 10 ml of ethanol, 8 ml of a 2M potassium carbonate aqueous solution, and 0.13 g of tetrakis(triphenylphosphine) palladium (0) were added, and the mixture was heated to reflux for 10 hours while being stirred. The mixture was cooled to reach room temperature, separated, dehydrated with anhydrous magnesium sulfate, and then concentrated to obtain a crude product. The crude product was purified by column chromatography (carrier: silica gel, eluent: ethyl acetate/n-hexane=2/1) to obtain 2.3 g (yield: 63%) of 3-(m-{m-[5-(dibenzofuran-4-yl)-3-{m-[m-(3-pyridyl) phenyl] phenyl} phenyl] phenyl} phenyl) pyridine (Compound 19) as a white powder.

The structure of the obtained white powder was identified by using NMR. The 1H-NMR measurement results are shown in FIG. 4.

The following 34 hydrogen signals were detected by 1H-NMR (DMSO-d6). δ (ppm)=9.02 (2H), 8.58-8.59 (2H), 8.26 (4H), 8.19-8.22 (5H), 8.12 (2H), 7.93-7.96 (3H), 7.84-7.89 (4H), 7.74-7.76 (2H), 7.54-7.69 (6H), 7.40-7.49 (4H).

### Example 5

### Synthesis of 5-(m-{m-[3-(3-pyridyl)-5-{m[m-(5-pyrimidinyl) phenyl] phenyl} phenyl] phenyl} phenyl) pyrimidine (Compound 6)

To a reaction vessel in which the atmosphere was replaced with nitrogen gas, 20.9 g of 3,3′-dibromobiphenyl, 6.4 g of 5-pyrimidineboronic acid, 160 ml of 1,4-dioxane, 38 ml of a 2M potassium carbonate aqueous solution, and 1.2 g of tetrakis(triphenylphosphine) palladium (0) were added, and the mixture was heated to reflux for 4 hours while being stirred. The mixture was cooled to reach room temperature, separated, dehydrated with anhydrous magnesium sulfate, and then concentrated to obtain a crude product. The crude product was purified by column chromatography (carrier: silica gel, eluent: ethyl acetate/n-hexane=1/2) to obtain 7.3 g (yield: 46%) of 5-(3′-bromobiphenyl-3-yl) pyrimidine as a white powder.

To a reaction vessel in which the atmosphere was replaced with nitrogen gas, 11.0 g of 5-(3′-bromobiphenyl-3-yl) pyrimidine, 9.9 g of bis(pinacolato) diboron, 5.2 g of potassium acetate, 100 ml of 1,4-dioxane, and 0.3 g of a [1,1′-bis (diphenylphosphino) ferrocene] dichloropalladium (II) dichloromethane adduct were added, and the mixture was heated at 90° C. for 5.5 hours while being stirred. The mixture was cooled to reach room temperature, separated, dehydrated with anhydrous sodium sulfate, and then concentrated to obtain a crude product. The crude product was purified by column chromatography (carrier: silica gel, eluent: ethyl acetate/n-hexane=1/3) to obtain 8.3 g (yield: 66%) of 5-[3 (4,4,5,5-tetramethyl-1,3,2-dioxaborolan-2-yl) biphenyl-3-yl] pyrimidine as a white powder.

To a reaction vessel in which the atmosphere was replaced with nitrogen gas, 1.5 g of 3-(3,5-dibromophenyl) pyridine, 3.6 g of 5-[3′-(4,4,5,5-tetramethyl-1,3,2-dioxaborolan-2-yl) biphenyl-3-yl] pyrimidine, 100 ml of 1,4-dioxane, 10 ml of a 2M potassium carbonate aqueous solution, and 0.12 g of tetrakis(triphenylphosphine) palladium (0) were added, and the mixture was heated to reflux for 15.5 hours while being stirred. The mixture was cooled to reach room temperature, separated, dehydrated with anhydrous sodium sulfate, and then concentrated to obtain a crude product. The crude product was purified by column chromatography (carrier: silica gel, eluent: ethyl acetate) to obtain 1.9 g (yield: 63%) of 5-(m-{m-[3-(3-pyridyl)-5-{m-[m-(5-pyrimidinyl) phenyl] phenyl} phenyl] phenyl} phenyl) pyrimidine (Compound 6) as a white powder.

The structure of the obtained white powder was identified by using NMR. The 1H-NMR measurement results are shown in FIG. 5.

The following 29 hydrogen signals were detected by 1H-NMR (CDCl₃). δ (ppm)=9.24 (2H), 9.02 (4H), 8.99 (1H), 8.65-8.67 (1H), 8.00-8.03 (1H), 7.92-7.95 (3H), 7.84-7.86 (4H), 7.73-7.80 (4H), 7.58-7.72 (8H), 7.42-7.45 (1H).

### Example 6

### Synthesis of 5-(m-{m-[3-(3-pyridyl)-5-{m[m-(3-pyridyl) phenyl] phenyl} phenyl] phenyl} phenyl) pyrimidine (Compound 194)

To a reaction vessel in which the atmosphere was replaced with nitrogen gas, 3.1 g of 3-(3,5-dibromophenyl) pyridine, 2.7 g of 5-[3′-(4,4,5,5-tetramethyl-1,3,2-dioxaborolan-2-yl) biphenyl-3-yl] pyrimidine, 80 ml of 1,4-dioxane, 6 ml of a 2M potassium carbonate aqueous solution, and 0.17 g of tetrakis(triphenylphosphine) palladium (0) were added, and the mixture was heated to reflux for 13 hours while being stirred. The mixture was cooled to reach room temperature, separated, dehydrated with anhydrous sodium sulfate, and then concentrated to obtain a crude product. The crude product was purified by column chromatography (carrier: silica gel, eluent: ethyl acetate) to obtain 1.9 g (yield: 55%) of 5-(m-{m-[5-bromo-3-(3-pyridyl) phenyl] phenyl} phenyl) pyrimidine as a white powder.

To a reaction vessel in which the atmosphere was replaced with nitrogen gas, 1.8 g of 5-(m-{m-[5-bromo-3-(3-pyridyl) phenyl] phenyl} phenyl) pyrimidine, 1.7 g of 3-[3′-(4,4,5,5-tetramethyl-1,3,2-dioxaborolan-2-yl) biphenyl-3-yl] pyridine, 50 ml of 1,4-dioxane, 3 ml of a 2M potassium carbonate aqueous solution, and 0.10 g of tetrakis (triphenylphosphine) palladium (0) were added, and the mixture was heated to reflux for 18 hours while being stirred. The mixture was cooled to reach room temperature, separated, dehydrated with anhydrous sodium sulfate, and then concentrated to obtain a crude product. The crude product was purified by column chromatography (carrier: silica gel, eluent: ethyl acetate) to obtain 2.1 g (yield: 88%) of 5-(m-{m-[3-(3-pyridyl)-5-{m-[m-(3-pyridyl) phenyl] phenyl} phenyl] phenyl} phenyl) pyrimidine (Compound 194) as a white powder.

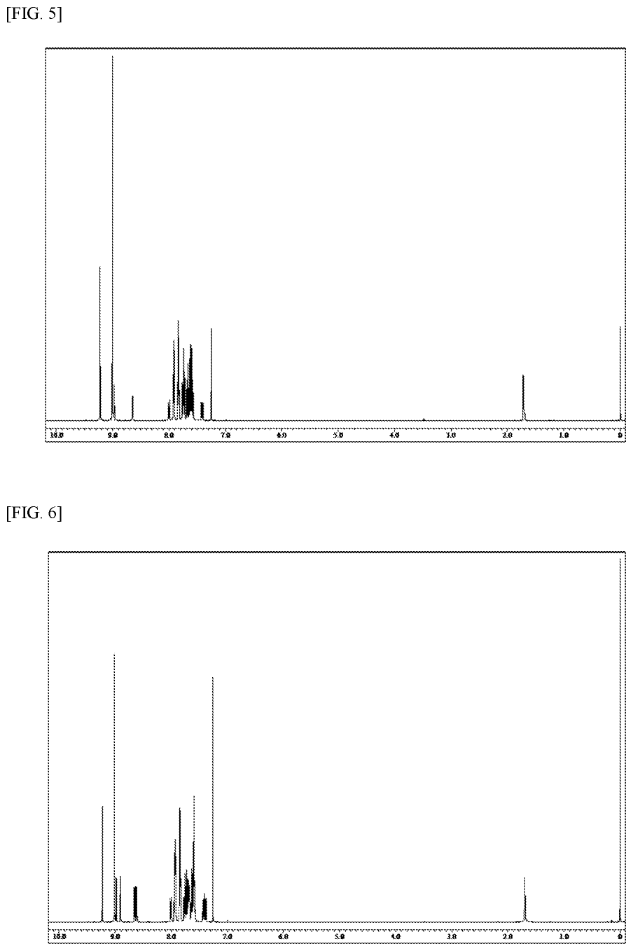

The structure of the obtained white powder was identified by using NMR. The 1H-NMR measurement results are shown in FIG. 6.

The following 30 hydrogen signals were detected by 1H-NMR (CDCl₃). δ (ppm)=9.23 (1H), 9.02 (2H), 8.98 (1H), 8.91 (1H), 8.64-8.68 (1H), 8.60-8.64 (1H), 7.98-8.03 (1H), 7.90-7.96 (4H), 7.81-7.87 (4H), 7.55-7.80 (12H), 7.36-7.45 (2H).

### Example 7

A melting point and a glass transition point of the compounds of Examples were determined by a high-sensitivity differential scanning calorimeter (DSC6200, manufactured by Seiko Instruments Inc.).

| | Melting point | Glass transition point |
|---|---|---|
| Compound of Example 1 | N.O. | 83° C. |
| Compound of Example 2 | N.O. | 85° C. |
| Compound of Example 3 | N.O. | 96° C. |
| Compound of Example 4 | N.O. | 99° C. |
| Compound of Example 5 | N.O. | 106° C. |
| Compound of Example 6 | N.O. | 96° C. |

As can be seen from the above, the compounds of the above-mentioned Examples exhibit values equivalent to or higher than a glass transition point (83° C.) of BCP, which is a general hole blocking layer, and the compounds have good thin film stability.

### Example 8

A vapor-deposited film having a film thickness of 100 nm was formed on an ITO substrate by using the compounds of the above-mentioned Examples, and the work function of the film was measured by using an atmospheric photoelectron spectrometer (AC-3 type, manufactured by Riken Keiki Co., Ltd.).

| | Work Function |
|---|---|
| Compound of Example 1 | 6.50 eV |
| Compound of Example 2 | 6.47 eV |
| Compound of Example 3 | 6.59 eV |
| Compound of Example 4 | 6.39 eV |
| Compound of Example 5 | 6.59 eV |
| Compound of Example 6 | 6.57 eV |

As can be seen from the above, the compounds of the above-mentioned Examples exhibit energy levels equivalent to the work function of a general hole blocking layer such as TAZ or BCP, and have a good hole blocking ability.

### Example 9

A vapor-deposited film having a film thickness of 50 nm was formed on a quartz substrate by using the compounds of the above-mentioned Examples, an ultraviolet-visible absorption spectrum was measured by using a commercially available spectrophotometer, and a band gap was calculated based on a wavelength of an absorption edge on a long wavelength side. In addition, the electron affinity was calculated from the values of the work function and the band gap.

| | Band gap | Electron affinity |
|---|---|---|
| Compound of Example 1 | 4.10 eV | 2.40 eV |
| Compound of Example 2 | 4.07 eV | 2.40 eV |
| Compound of Example 3 | 4.02 eV | 2.57 eV |
| Compound of Example 4 | 3.69 eV | 2.70 eV |
| Compound of Example 5 | 3.98 eV | 2.61 eV |
| Compound of Example 6 | 4.06 eV | 2.51 eV |
| Alq₃ | 2.70 eV | 3.00 eV |
| TPBi | 3.50 eV | 2.70 eV |

As can be seen from the above, the compounds of the above-mentioned Examples exhibit a wider value than the band gap of a general electron transport material such as Alq₃ or TPBi, have a small electron affinity, and have good electron injection properties particularly with respect to a blue light emitting layer.

### Example 10

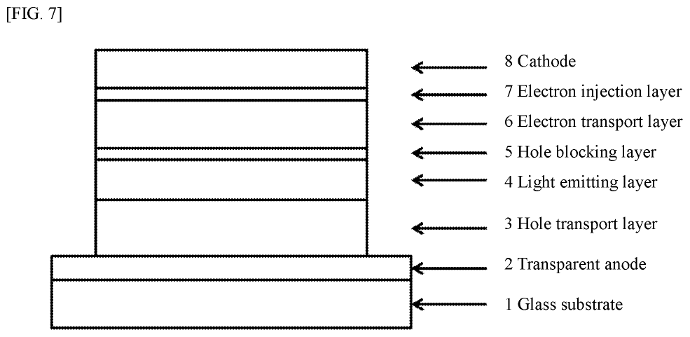

As shown in FIG. 7, the organic EL device in the present Example was prepared by forming an ITO electrode as a transparent anode 2 on a glass substrate 1 in advance, and depositing a hole transport layer 3, a light emitting layer 4, a hole blocking layer 5, an electron transport layer 6, an electron injection layer 7, and a cathode (aluminum electrode) 8 in this order on the ITO electrode.

Specifically, the organic EL device was produced by the following procedure. The glass substrate 1 on which an ITO having a film thickness of 150 nm was formed was washed with an organic solvent, and then the surface thereof was cleaned by UV ozone treatment. Thereafter, the glass substrate with the ITO electrode was mounted in a vacuum vapor deposition apparatus, followed by evacuating to 0.001 Pa or lower. Subsequently, TAPC was vapor-deposited on the transparent anode 2 at a vapor deposition rate of 1.0 Å/s to form the hole transport layer 3 covering the transparent anode 2 in a film thickness of 40 nm. On the hole transport layer 3, mCP and a blue phosphorescent material Flrpic were subjected to a binary deposition at a deposition rate ratio of mCP:FIrpic=94:6 to form the light emitting layer 4 in a film thickness of 30 nm. On the light emitting layer 4, the compound (Compound 1) of Example 1 was deposited at a deposition rate of 1.0 Å/s to form the hole blocking layer 5 in a film thickness of 5 nm. On the hole blocking layer 5, TPBi was deposited at a deposition rate of 1.0 Å/s to form the electron transport layer 6 in a film thickness of 40 nm. On the electron transport layer 6, lithium fluoride was deposited at a deposition rate of 0.1 Å/s to form the electron injection layer 7 in a film thickness of 0.5 nm. Finally, aluminum was deposited in a film thickness of 150 nm to form the cathode 8. The prepared organic EL device was measured for properties in the atmosphere at normal temperature.

The results of measuring the light emission properties upon applying a current having a current density of 10 mA/cm² to the organic EL device produced by using the compound (Compound 1) of Example 1 were shown in Table 1.

### Example 11

An organic EL device was produced in the same manner as in Example 10 except that the compound (Compound 4) of Example 2 was used instead of the material of the hole blocking layer 5 in Example 10. The results of measuring the light emission properties upon applying a current having a current density of 10 mA/cm² to the produced organic EL device were shown in Table 1.

### Example 12

An organic EL device was produced in the same manner as in Example 10 except that the compound (Compound 7) of Example 3 was used instead of the material of the hole blocking layer 5 in Example 10. The results of measuring the light emission properties upon applying a current having a current density of 10 mA/cm² to the produced organic EL device were shown in Table 1.

### Example 13

An organic EL device was produced in the same manner as in Example 10 except that the compound (Compound 19) of Example 4 was used instead of the material of the hole blocking layer 5 in Example 10. The results of measuring the light emission properties upon applying a current having a current density of 10 mA/cm² to the produced organic EL device were shown in Table 1.

### Example 14

An organic EL device was produced in the same manner as in Example 10 except that the compound (Compound 6) of Example 5 was used instead of the material of the hole blocking layer 5 in Example 10. The results of measuring the light emission properties upon applying a current having a current density of 10 mA/cm² to the produced organic EL device were shown in Table 1.

### Example 15

An organic EL device was produced in the same manner as in Example 10 except that the compound (Compound 194) of Example 6 was used instead of the material of the hole blocking layer 5 in Example 10. The results of measuring the light emission properties upon applying a current having a current density of 10 mA/cm² to the produced organic EL device were shown in Table 1.

### Example 16

An organic EL device was produced in the same manner as in Example 10 except that the compound (Compound 1) of Example 1 was used instead of the materials of the hole blocking layer 5 and the electron transport layer 6 in Example 10. The results of measuring the light emission properties upon applying a current having a current density of 10 mA/cm² to the produced organic EL device were shown in Table 1.

### Example 17

An organic EL device was produced in the same manner as in Example 10 except that the compound (Compound 4) of Example 2 was used instead of the materials of the hole blocking layer 5 and the electron transport layer 6 in Example 10. The results of measuring the light emission properties upon applying a current having a current density of 10 mA/cm² to the produced organic EL device were shown in Table 1.

### Example 18

An organic EL device was produced in the same manner as in Example 10 except that the compound (Compound 7) of Example 3 was used instead of the materials of the hole blocking layer 5 and the electron transport layer 6 in Example 10. The results of measuring the light emission properties upon applying a current having a current density of 10 mA/cm² to the produced organic EL device were shown in Table 1.

### Example 19

An organic EL device was produced in the same manner as in Example 10 except that the compound (Compound 6) of Example 5 was used instead of the materials of the hole blocking layer 5 and the electron transport layer 6 in Example 10. The results of measuring the light emission properties upon applying a current having a current density of 10 mA/cm² to the produced organic EL device were shown in Table 1.

### Example 20

An organic EL device was produced in the same manner as in Example 10 except that the compound (Compound 194) of Example 6 was used instead of the materials of the hole blocking layer 5 and the electron transport layer 6 in Example 10. The results of measuring the light emission properties upon applying a current having a current density of 10 mA/cm² to the produced organic EL device were shown in Table 1.

### Comparative Example 1

For comparison, an organic EL device was produced in the same manner as in Example 10 except that TPBi was used instead of the material of the hole blocking layer 5 in Example 10. The results of measuring the light emission properties upon applying a current having a current density of 10 mA/cm² to the produced organic EL device were shown in Table 1.

TABLE 1

| | | | | | Luminous | Power |
|---|---|---|---|---|---|---|
| | Hole | Electron | Voltage | Luminance | efficiency | efficiency |
| | blocking | transport | [V] | [cd/m²] | [cd/A] | [lm/W] |
| | layer 5 | layer 6 | (@10 mA/cm²) | (@10 mA/cm²) | (@10 mA/cm²) | (@10 mA/cm²) |
| Example 10 | Compound 1 | TPBi | 8.5 | 1255 | 12.5 | 4.6 |
| Example 11 | Compound 4 | TPBi | 7.5 | 1628 | 16.3 | 6.7 |
| Example 12 | Compound 7 | TPBi | 7.6 | 1851 | 19.2 | 9.9 |
| Example 13 | Compound 19 | TPBi | 7.4 | 1308 | 12.9 | 5.3 |
| Example 14 | Compound 6 | TPBi | 6.8 | 2200 | 23.3 | 10.8 |
| Example 15 | Compound 194 | TPBi | 6.5 | 2481 | 22.5 | 10.7 |
| Example 16 | Compound 1 | Compound 1 | 11.6 | 2010 | 20.1 | 5.5 |
| Example 17 | Compound 4 | Compound 4 | 9.7 | 2050 | 20.5 | 6.5 |
| Example 18 | Compound 7 | Compound 7 | 7.6 | 1540 | 16.5 | 7.1 |
| Example 19 | Compound 6 | Compound 6 | 9.0 | 1847 | 19.2 | 6.8 |
| Example 20 | Compound 194 | Compound 194 | 8.3 | 2933 | 26.9 | 9.8 |
| Comparative | TPBi | TPBi | 9.0 | 1276 | 12.4 | 4.3 |
| Example 1 | | | | | | |

As shown in Table 1, luminous efficiencies upon applying a current having a current density of 10 mA/cm² were increased to 12.5 cd/A in Example 10 (Compound 1 was used as a hole blocking layer), 16.3 cd/A in Example 11 (Compound 4 was used as a hole blocking layer), 19.2 cd/A in Example 12 (Compound 7 was used as a hole blocking layer), 12.9 cd/A in Example 13 (Compound 19 was used as a hole blocking layer), 23.3 cd/A in Example 14 (Compound 6 was used as a hole blocking layer), 22.5 cd/A in Example 15 (Compound 194 was used as a hole blocking layer), 20.1 cd/A in Example 16 (Compound 1 was used as a hole blocking layer-cum-electron transport layer), 20.5 cd/A in Example 17 (Compound 4 was used as a hole blocking layer-cum-electron transport layer), 16.5 cd/A in Example 18 (Compound 7 was used as a hole blocking layer-cum-electron transport layer), 19.2 cd/A in Example 19 (Compound 6 was used as a hole blocking layer-cum-electron transport layer), and 26.9 cd/A in Example 20 (Compound 194 was used as a hole blocking layer-cum-electron transport layer), with respect to 12.4 cd/A in Comparative Example 1.

As shown in Table 1, power efficiencies upon applying a current having a current density of 10 mA/cm² were increased to 4.6 lm/W in Example 10, 6.7 lm/W in Example 11, 9.9 lm/W in Example 12, 5.3 lm/W in Example 13, 10.8 lm/W in Example 14, 10.7 lm/W in Example 15, 5.5 lm/W in Example 16, 6.5 lm/W in Example 17, 7.1 lm/W in Example 18, 6.8 lm/W in Example 19, and 9.8 lm/W in Example 20, with respect to 4.3 lm/W of Comparative

### Example 1

As is clear from these results, it was found that the organic EL device using the compounds of the present embodiment can achieve improvements in luminous efficiency and power efficiency as compared with a device using TPBi used as a general electron transport material.

Although the present invention has been described in detail with reference to specific embodiments, it is apparent to those skilled in the art that various changes and modifications can be made without departing from the spirit and scope of the present invention.

The present application is based on Japanese Patent Application (No. 2019-42267) filed on Mar. 8, 2019, the entire contents of which are incorporated herein by reference. In addition, all references cited here are entirely incorporated.

### INDUSTRIAL APPLICABILITY

The trisubstituted benzene compound that has two aromatic substituents having a nitrogen-containing heterocycle at a terminal thereof and further has another aromatic substituent, of the present invention is excellent as a compound for an organic EL device because the electron injection and transport performance is good and the band gap is wide. When an organic EL device is produced by using the compound, high efficiency can be obtained and durability can be improved. For example, it is possible to expand to applications for home appliances and lighting.

### REFERENCES SIGNS LIST

1 Glass substrate
2 Transparent anode
3 Hole transport layer
4 Light emitting layer
5 Hole blocking layer
6 Electron transport layer
7 Electron injection layer
8 Cathode

1: A trisubstituted benzene compound represented by the following general formulas (1) and (2): 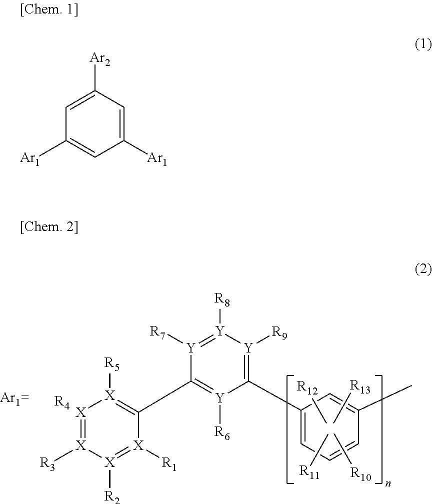
(in the formula, Ar₂ is different from Ar₁, and is an optionally substituted monovalent aromatic hydrocarbon group or aromatic heterocyclic group;
R₁ to R₁₃, which may be the same as or different from each other, each represent a hydrogen atom, a deuterium atom, a fluorine atom, a chlorine atom, a cyano group, a trifluoromethyl group, a linear or branched alkyl group having a carbon number of 1 to 8 which may have a substituent, a cycloalkyl group having a carbon number of 5 to 10 which may have a substituent, a linear or branched alkenyl group having a carbon number of 2 to 6 which may have a substituent, a linear or branched alkyloxy group having a carbon number of 1 to 6 which may have a substituent, or a cycloalkyloxy group having a carbon number of 5 to 10 which may have a substituent;
X's and Y's in Ar₁ each represent a carbon atom or a nitrogen atom, and at least one X represents a nitrogen atom;
in Ar₁, n represents an integer of 1 to 3; and
two Ar₁'s may be the same as or different from each other).

2: The trisubstituted benzene compound according to claim 1, wherein Ar₁ in the general formula (1) is a substituent represented by any one of the following general formulas (3) to (6). 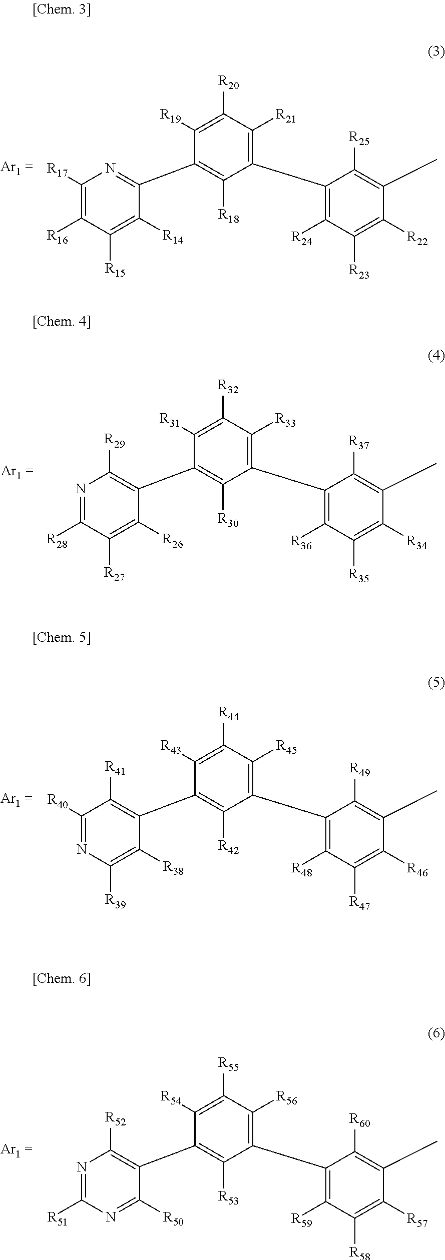
(in the formulas, R₁₄ to R₆₀, which may be the same as or different from each other, each represent a hydrogen atom, a deuterium atom, a fluorine atom, a chlorine atom, a cyano group, a trifluoromethyl group, a linear or branched alkyl group having a carbon number of 1 to 8 which may have a substituent, a cycloalkyl group having a carbon number of 5 to 10 which may have a substituent, a linear or branched alkenyl group having a carbon number of 2 to 6 which may have a substituent, a linear or branched alkyloxy group having a carbon number of 1 to 6 which may have a substituent, or a cycloalkyloxy group having a carbon number of 5 to 10 which may have a substituent).

3: An organic electroluminescence device comprising a pair of electrodes and at least one organic layer interposed therebetween, wherein the at least one organic layer comprises the trisubstituted benzene compound as described in claim 1 as a constituent material.

4: The organic electroluminescence device according to claim 3, wherein at least one layer of the organic layer containing the trisubstituted benzene compound is an electron transport layer.

5: The organic electroluminescence device according to claim 3, wherein at least one layer of the organic layer containing the trisubstituted benzene compound is a hole blocking layer.

6: The organic electroluminescence device according to claim 3, wherein at least one layer of the organic layer containing the trisubstituted benzene compound is an electron injection layer.

7: The organic electroluminescence device according to claim 3, wherein at least one layer of the organic layer containing the trisubstituted benzene compound is a hole injection layer.

8: The organic electroluminescence device according to claim 3, wherein at least one layer of the organic layer containing the trisubstituted benzene compound is a light emitting layer.
